SEQUENCE LISTING (126 sequences)

SEQ ID NO: 1 - protein, 25 aa, synthetic construct
  REPEAT 1..25: note = This sequence may encompass 1-5 "Gly Gly Gly Gly Ser" repeating units
GGGGSGGGGSGGGGSGGGGSGGGGS

SEQ ID NO: 2 - protein, 5 aa, synthetic construct
GGGGS

SEQ ID NO: 3 - protein, 10 aa, synthetic construct
GGGGSGGGGS

SEQ ID NO: 4 - protein, 15 aa, synthetic construct
GGGGSGGGGSGGGGS

SEQ ID NO: 5 - protein, 20 aa, synthetic construct
GGGGSGGGGSGGGGSGGGGS

SEQ ID NO: 6 - protein, 21 aa, synthetic construct
GGGGGSGGGGSGGGGSGGGGS

SEQ ID NO: 7 - protein, 21 aa, synthetic construct
GGGGSGGGGSGGGGSGGGGAS

SEQ ID NO: 8 - protein, 5 aa, synthetic construct
DGGGS

SEQ ID NO: 9 - protein, 5 aa, synthetic construct
TGEKP

SEQ ID NO: 10 - protein, 4 aa, synthetic construct
GGRR

SEQ ID NO: 11 - protein, 14 aa, synthetic construct
EGKSSGSGSESKVD

SEQ ID NO: 12 - protein, 18 aa, synthetic construct
KESGSVSSEQLAQFRSLD

SEQ ID NO: 13 - protein, 8 aa, synthetic construct
GGRRGGGS

SEQ ID NO: 14 - protein, 9 aa, synthetic construct
LRQRDGERP

SEQ ID NO: 15 - protein, 12 aa, synthetic construct
LRQKDGGGSERP

SEQ ID NO: 16 - protein, 18 aa, synthetic construct
GSTSGSGKPGSGEGSTKG

SEQ ID NO: 17 - protein, 5 aa, synthetic construct
EPKSC

SEQ ID NO: 18 - protein, 10 aa, synthetic construct
EPKSCGGGGS

SEQ ID NO: 19 - protein, 10 aa, synthetic construct
EPKSCDKTHT

SEQ ID NO: 20 - protein, 15 aa, synthetic construct
EPKSCDKTHTGGGGS

SEQ ID NO: 21 - protein, 5 aa, synthetic construct
DKTHT

SEQ ID NO: 22 - protein, 10 aa, synthetic construct
DKTHTGGGGS

SEQ ID NO: 23 - protein, 275 aa, synthetic construct
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 24 - protein, 99 aa, synthetic construct [sequence omitted]

SEQ ID NO: 25 - protein, 7 aa, synthetic construct
SSYYYMC

SEQ ID NO: 26 - protein, 18 aa, synthetic construct
CIYAGSSGSTYYASWAKG

SEQ ID NO: 27 - protein, 11 aa, synthetic construct
GAGYGNGGHSL

SEQ ID NO: 28 - protein, 11 aa, synthetic construct
RASKNIYNSLA

SEQ ID NO: 29 - protein, 7 aa, synthetic construct
GASNLES

SEQ ID NO: 30 - protein, 13 aa, synthetic construct
QATYYGHDNIGGA

SEQ ID NO: 31 - protein, 122 aa, synthetic construct
EVQLVESGGGSVQPGGSLRLSCAASGIDFSSYYYMCWVRQAPGKCLEWIGCIYAGSSGSTYYASWAKGRFTISRDNSKNTLYLQMNSLRAEDTATYYCARGAGYGNGGHSLWGQGTTVTVSS

SEQ ID NO: 32 - protein, 111 aa, synthetic construct
DIQMTQSPSTLSASVGDRVTITCRASKNIYNSLAWYQQKPGKAPKLLIYGASNLESGVPSRFSGSGSGTDYTLTISSLQPEDSATYYCQATYYGHDNIGGAFGCGTKVEIK

SEQ ID NO: 33 - protein, 253 aa, synthetic construct
DIQMTQSPSTLSASVGDRVTITCRASKNIYNSLAWYQQKPGKAPKLLIYGASNLESGVPSRFSGSGSGTDYTLTISSLQPEDSATYYCQATYYGHDNIGGAFGCGTKVEIKGGGGSGGGGSGGGGSGGGGSEVQLVESGGGSVQPGGSLRLSCAASGIDFSSYYYMCWVRQAPGKCLEWIGCIYAGSSGSTYYASWAKGRFTISRDNSKNTLYLQMNSLRAEDTATYYCARGAGYGNGGHSLWGQGTTVTVSS

SEQ ID NO: 34 - protein, 228 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 35 - protein, 215 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 37 - protein, 110 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLG

SEQ ID NO: 38 - protein, 6 aa, synthetic construct
STYAMN

SEQ ID NO: 39 - protein, 19 aa, synthetic construct
RIRSKYNNYATYYADSVKG

SEQ ID NO: 40 - protein, 14 aa, synthetic construct
HGNFGDSYVSWFAY

SEQ ID NO: 41 - protein, 14 aa, synthetic construct
GSSTGAVTTSNYAN

SEQ ID NO: 42 - protein, 7 aa, synthetic construct
GTNKRAP

SEQ ID NO: 43 - protein, 9 aa, synthetic construct
ALWYSNHWV

SEQ ID NO: 44 - protein, 487 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEIVMTQSPSTLSASVGDRVIITCQASENIYNSLAWYQQKPGKAPKLLIYGASNLESGVPSRFSGSGSGTEYTLTISSLQPDDFATYYCQSTYYGHDNVGGAFGCGTKLTVLGGGGGSGGGGSGGGGSGGGGSEVQLVESGGGSVQPGGSLRLSCTVSGIDFSSYYYMCWVRQAPGKCLEWIGCIYAGSSGSTYYASWAKGRFTISKDTSKNTVYLQMNSLRAEDTATYYCARGAGYGNDGHSLWGQGTTVTVSS

SEQ ID NO: 45 - protein, 215 aa, synthetic construct; identical to SEQ ID NO: 35

SEQ ID NO: 46 - protein, 487 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEIVMTQSPSTLSASVGDRVIITCQASENIYNSLAWYQQKPGKAPKLLIYGASNLESGVPSRFSGSGSGTEFTLTISSLQPDDFATYYCQSTYYGHDNVGGAFGCGTKLTVLGGGGGSGGGGSGGGGSGGGGSEVQLVESGGGSVQPGGSLRLSCTVSGIDFSSYYYMCWVRQAPGKCLEWIGCIYAGSSGSTYYASWAKGRFTISKDTSKNTVYLQMNSLRAEDTATYYCARGAGYGNDGHSLWGQGTTVTVSS

SEQ ID NO: 47 - protein, 215 aa, synthetic construct; identical to SEQ ID NO: 35

SEQ ID NO: 48 - protein, 486 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQQQLEESGGDLVKPGASLTLTCKASGIDFSSYYYMCWVRQAPGKGLEWIACIYAGSSGSTYYASWAKGRFTISKTSSTMVTLQMTSLTAADTATYFCARGAGYGNDGHSLWGPGTLVTVSSGGGGSGGGGSGGGGSGGGGSSDPMLTQTPASVSAAVGGTVTIKCQASENIYNSLAWYQQKPGQRPKLLIYGASNLESGVPSRFKGSGSGTEYTLTISDLEADDAATYYCQSTYYGHDNVGGAFGGGTEVVVR

SEQ ID NO: 49 - protein, 215 aa, synthetic construct; identical to SEQ ID NO: 35

SEQ ID NO: 50 - protein, 486 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSTLSASVGDRVTITCRASKNIYNSLAWYQQKPGKAPKLLIYGASNLESGVPSRFSGSGSGTDYTLTISSLQPEDFATYYCQSTYYGHDNVGGAFGCGTKVEIKGGGGSGGGGSGGGGSGGGGSEVQLVESGGGSVQPGGSLRLSCAASGIDFSSYYYMCWVRQAPGKCLEWIGCIYAGSSGSTYYASWAKGRFTISRDNSKNTLYLQMNSLRAEDTATYYCARGAGYGNDGHSLWGQGTTVTVSS

SEQ ID NO: 51 - protein, 215 aa, synthetic construct; identical to SEQ ID NO: 35

SEQ ID NO: 52 - protein, 486 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIQMTQSPSTLSASVGDRVTITCRASKNIYNSLAWYQQKPGKAPKLLIYGASNLESGVPSRFSGSGSGTDYTLTISSLQPEDSATYYCQATYYGHDNIGGAFGCGTKVEIKGGGGSGGGGSGGGGSGGGGSEVQLVESGGGSVQPGGSLRLSCAASGIDFSSYYYMCWVRQAPGKCLEWIGCIYAGSSGSTYYASWAKGRFTISRDNSKNTLYLQMNSLRAEDTATYYCARGAGYGNGGHSLWGQGTTVTVSS

SEQ ID NO: 53 - protein, 473 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIQMTQSPSTLSASVGDRVTITCRASKNIYNSLAWYQQKPGKAPKLLIYGASNLESGVPSRFSGSGSGTDYTLTISSLQPEDSATYYCQATYYGHDNIGGAFGCGTKVEIKGGGGSGGGGSGGGGSGGGGSEVQLVESGGGSVQPGGSLRLSCAASGIDFSSYYYMCWVRQAPGKCLEWIGCIYAGSSGSTYYASWAKGRFTISRDNSKNTLYLQMNSLRAEDTATYYCARGAGYGNGGHSLWGQGTTVTVSS

SEQ ID NO: 54 - protein, 9 aa, synthetic construct
NTDNNLAVY

SEQ ID NO: 55 - protein, 9 aa, synthetic construct
NTDNLLTEY

SEQ ID NO: 56 - protein, 9 aa, synthetic construct
ETDNNIVVY

SEQ ID NO: 57 - protein, 9 aa, synthetic construct
YTDNWLAVY

SEQ ID NO: 58 - protein, 9 aa, synthetic construct
PTDENLARY

SEQ ID NO: 59 - protein, 9 aa, synthetic construct
NSDNNTIFV

SEQ ID NO: 60 - protein, 9 aa, synthetic construct
NLDKNLIKY

SEQ ID NO: 61 - protein, 9 aa, synthetic construct
NSDSNLTTY

SEQ ID NO: 62 - protein, 9 aa, synthetic construct
TADHNLLLY

SEQ ID NO: 63 - protein, 9 aa, synthetic construct
QLDNQLDAY

SEQ ID NO: 64 - protein, 9 aa, synthetic construct
SVDSNLFVY

SEQ ID NO: 65 - protein, 9 aa, synthetic construct
SADNNIVLY

SEQ ID NO: 66 - protein, 9 aa, synthetic construct
RTERNVAVY

SEQ ID NO: 67 - protein, 9 aa, synthetic construct
GGDNQLLLY

SEQ ID NO: 68 - protein, 9 aa, synthetic construct
NLDQSLAHY

SEQ ID NO: 69 - protein, 9 aa, synthetic construct
FTDNQILLK

SEQ ID NO: 70 - protein, 9 aa, synthetic construct
GADRNLLVY

SEQ ID NO: 71 - protein, 9 aa, synthetic construct
RTEEALQLY

SEQ ID NO: 72 - protein, 9 aa, synthetic construct
FLHHTLALY

SEQ ID NO: 73 - protein, 9 aa, synthetic construct
VTQESKAVY

SEQ ID NO: 74 - protein, 9 aa, synthetic construct
MSEEHTAVY

SEQ ID NO: 75 - protein, 9 aa, synthetic construct
RTDGILALY

SEQ ID NO: 76 - protein, 9 aa, synthetic construct
DTDFVNEFY

SEQ ID NO: 77 - protein, 9 aa, synthetic construct
LSSLASSRY

SEQ ID NO: 78 - protein, 9 aa, synthetic construct
ATDSLNNEY

SEQ ID NO: 79 - protein, 9 aa, synthetic construct
NVDYGVPFY

SEQ ID NO: 80 - protein, 9 aa, synthetic construct
ELARDSIYY

SEQ ID NO: 81 - protein, 9 aa, synthetic construct
YTDVSNMSH

SEQ ID NO: 82 - protein, 9 aa, synthetic construct
NSEEHSARY

SEQ ID NO: 83 - protein, 9 aa, synthetic construct
EVDPTSNTY

SEQ ID NO: 84 - protein, 9 aa, synthetic construct
ISERILSTY

SEQ ID NO: 85 - protein, 9 aa, synthetic construct
QVDYYGLYY

SEQ ID NO: 86 - protein, 10 aa, synthetic construct
YTTNDSSTAY

SEQ ID NO: 87 - protein, 10 aa, synthetic construct
NSNSSYYGKY

SEQ ID NO: 88 - protein, 10 aa, synthetic construct
ILDTAGLEEY

SEQ ID NO: 89 - protein, 10 aa, synthetic construct
SLEDPSTDYY

SEQ ID NO: 90 - protein, 10 aa, synthetic construct
TTDPSFLGRY

SEQ ID NO: 91 - protein, 10 aa, synthetic construct
YTNSFTRGVY

SEQ ID NO: 92 - protein, 10 aa, synthetic construct
VSDTENTHIY

SEQ ID NO: 93 - protein, 10 aa, synthetic construct
RSDSGQQARY

SEQ ID NO: 94 - protein, 9 aa, synthetic construct
ATDNNLAVY

SEQ ID NO: 95 - protein, 9 aa, synthetic construct
NADNNLAVY

SEQ ID NO: 96 - protein, 9 aa, synthetic construct
NTANNLAVY

SEQ ID NO: 97 - protein, 9 aa, synthetic construct
NTDANLAVY

SEQ ID NO: 98 - protein, 9 aa, synthetic construct
NTDNALAVY

SEQ ID NO: 99 - protein, 9 aa, synthetic construct
NTDNNAAVY

SEQ ID NO: 100 - protein, 9 aa, synthetic construct
NTDNNLSVY

SEQ ID NO: 101 - protein, 9 aa, synthetic construct
NTDNNLAAY

SEQ ID NO: 102 - protein, 9 aa, synthetic construct
NTDNNLAVA

SEQ ID NO: 103 - protein, 9 aa, synthetic construct
RTDNNLAVY

SEQ ID NO: 104 - protein, 9 aa, synthetic construct
NRDNNLAVY

SEQ ID NO: 105 - protein, 9 aa, synthetic construct
NTRNNLAVY

SEQ ID NO: 106 - protein, 9 aa, synthetic construct
NTDRNLAVY

SEQ ID NO: 107 - protein, 9 aa, synthetic construct
NTDNRLAVY

SEQ ID NO: 108 - protein, 9 aa, synthetic construct
NTDNNRAVY

SEQ ID NO: 109 - protein, 9 aa, synthetic construct
NTDNNLRVY

SEQ ID NO: 110 - protein, 9 aa, synthetic construct
NTDNNLARY

SEQ ID NO: 111 - protein, 9 aa, synthetic construct
NTDNNLAVR

SEQ ID NO: 112 - protein, 9 aa, synthetic construct
DTDNNLAVY

SEQ ID NO: 113 - protein, 9 aa, synthetic construct
NDDNNLAVY

SEQ ID NO: 114 - protein, 9 aa, synthetic construct
NTSNNLAVY

SEQ ID NO: 115 - protein, 9 aa, synthetic construct
NTDDNLAVY

SEQ ID NO: 116 - protein, 9 aa, synthetic construct
NTDNDLAVY

SEQ ID NO: 117 - protein, 9 aa, synthetic construct
NTDNNDAVY

SEQ ID NO: 118 - protein, 9 aa, synthetic construct
NTDNNLDVY

SEQ ID NO: 119 - protein, 9 aa, synthetic construct
NTDNNLADY

SEQ ID NO: 120 - protein, 9 aa, synthetic construct
NTDNNLAVD

SEQ ID NO: 121 - protein, 9 aa, synthetic construct
NTDEWRAVY

SEQ ID NO: 122 - protein, 9 aa, synthetic construct
NTDRWEAVY

SEQ ID NO: 123 - protein, 9 aa, synthetic construct
NAWNNLEKY

SEQ ID NO: 124 - protein, 9 aa, synthetic construct
CTELKLSDY

SEQ ID NO: 125 - protein, 9 aa, synthetic construct
FTELTLGEF

SEQ ID NO: 126 - protein, 9 aa, synthetic construct
ASDRNIVLY

(19) United States
(12) Patent Application Publication
(10) Pub. No.: US 2026/0284218 A1
(43) Pub. Date: Sep. 24, 2026

(54) ANTIGEN BINDING PROTEINS TARGETING AN HLA-RESTRICTED KK-LC-1 PEPTIDE

(71) Applicant: CDR-LIFE AG, Horgen (CH)

(72) Inventors: Fabian Bert Scheifele, Horgen (CH); Anna Maria Sobieraj, Horgen (CH); Nagjie Laila Alijaj, Horgen (CH); Stephanie Jungmichel, Horgen (CH); Philip Knobel, Horgen (CH); Leonardo Borras, Horgen (CH)

(21) Appl. No.: 19/575,719

(22) Filed: Mar. 23, 2026

Related U.S. Application Data
(60) Provisional application No. 63/776,458, filed on Mar. 24, 2025.

(51) Int. Cl. A61K 47/68; A61K 38/00; A61K 49/00; A61P 35/00; C07K 14/725; C07K 16/28

(52) U.S. Cl. CPC A61K 47/6849; A61K 49/0002; A61P 35/00; C07K 14/7051; C07K 16/2833; A61K 38/00; C07K 2317/31; C07K 2317/35; C07K 2317/622

(57) ABSTRACT

Described herein are antigen binding proteins targeting KK-LC-1 derived peptide-MHCs (pMHCs). Also described are multispecific antigen binding proteins comprising an antigen binding domain with specificity to CD3, and at least one target peptide binding domain. Methods of treatment with the same are also described.

### RELATED APPLICATIONS

This application claims the benefit of U.S. Provisional Patent Application Ser. No. 63/776,458, filed Mar. 24, 2025, the entire disclosure of which is hereby incorporated herein by reference.

### SEQUENCE LISTING

The instant application contains a Sequence Listing which has been submitted electronically in XML format and is hereby incorporated by reference in its entirety. Said XML file, created on Mar. 19, 2026, is named 776115_CDR9-018_ST26.xml and is 115,678 bytes in size.

### FIELD OF THE DISCLOSURE

This disclosure relates to novel antigen binding proteins that bind to tumor peptide-MHC (pMHC) complexes with high specificity, having favorable properties for therapeutic purposes. Such pMHC binding proteins may be incorporated into CARs or further comprise a CD3 targeting moiety which provides efficient T-cell mediated cancer cell killing despite very low levels of pMHC on the cell surface.

### BACKGROUND

Kita-Kyushu lung cancer antigen-1 (KK-LC-1/CT83) is a cancer/testis antigen, a class of cancer antigens that are characterized by their spontaneous immunogenicity and unique expression pattern. Cancer/testis antigens are normally expressed only in the germ cells of the normal human testis and placenta but are also activated in tumor cells. The KK-LC-1 peptide NTDNNLAVY (SEQ ID NO: 54) is displayed on major histocompatibility complexes (MHCs) in a variety of solid tumor types including gastric, lung, breast, and cervical cancer, including many unmet need tumors, but not in normal tissue.

Peptide-MHC complexes (pMHCs) derived from intracellular tumor associated antigens (TAAs), such as KK-LC-1-derived NTDNNLAVY (SEQ ID NO: 54), represent a large repertoire of novel targets for immunotherapy. They have been traditionally targeted by TCR-engineered T cells or soluble recombinant T-cell receptors (TCRs) fused to an anti-CD3 fragment. However, therapeutic use of soluble TCRs is hampered by low target affinity and challenges related to expression. Naturally occurring cancer reactive TCRs typically exhibit low binding affinities for their pMHC targets. Therefore, substantial engineering efforts are needed to achieve better affinity as well as better biophysical properties to be developed as drugs which may compromise the required specificity to the pMHC target. Conversely, antibodies may exhibit affinities in the nanomolar range or even below. However, artificial antibodies do not pass through thymic selection which may dampen putative cross-reactivity of naturally occurring TCRs; hence, there remains an inherent challenge that TCR-like antibodies may cross-react with pMHCs presenting similar peptides, leading to undesired side-effects. Another obstacle to the development of immunotherapies involving TCRs or TCR-like antibodies is the low population coverage as the high level of polymorphism of the HLA genes results in a highly diverse number of pMHCs.

There is a growing number of agents targeting pMHCs, most of them are however restricted to HLA-A*02 which highlights the urgent need to identify effective pMHC-targeted therapies for solid tumors, especially on other alleles to increase the proportion of eligible patients

### SUMMARY

The present invention relates to antigen binding proteins which specifically bind to a major histocompatibility complex (MHC)-displayed KK-LC-1 peptide NTDNNLAVY (SEQ ID NO: 54) which address this clinical situation. The peptide is presented on HLAs of the A*01 supertype and is a promising tumor target as its expression is restricted to tumors and it is broadly expressed in many solid tumor types. However, targeting the KK-LC-1 presenting MHCs is challenging due to the high levels of similar off-target peptides presented in healthy tissues and its low antigenicity.

An adapted screening process, humanization and extensive specificity profiling throughout lead optimization was required to generate safe antigen binding proteins with drug-like properties. The antigen binding proteins of the disclosure show high specificity and achieve nanomolar affinity for KK-LC-1/HLA-A*01. The antigen binding proteins described herein can be manufactured at scale using e.g., CHO cells, and exhibit properties suitable for clinical development including high solubility, thermal stability and manufacturability.

In one aspect, the antigen binding protein specifically binding MHC-displayed NTDNNLAVY (SEQ ID NO: 54) comprises: (a) an antibody heavy chain variable (VH) domain comprising an HCDR1 amino acid sequence of SSYYYMC (SEQ ID NO: 25), an HCDR2 amino acid sequence of CIYAGSSGSTYYASWAKG (SEQ ID NO: 26), and an HCDR3 amino acid sequence of GAGYGNGGHSL (SEQ ID NO: 27); and (b) an antibody light chain variable (VL) domain comprising an LCDR1 amino acid sequence of RASKNIYNSLA (SEQ ID NO: 28), an LCDR2 amino acid sequence of GASNLES (SEQ ID NO: 29), and an LCDR3 amino acid sequence of QATYYGHDNIGGA (SEQ ID NO: 30).

In some embodiments, said antigen binding protein specifically targeting MHC-displayed NTDNNLAVY (SEQ ID NO: 54) comprises a variable light chain sequence comprising or consisting of SEQ ID NO: 32 or a variant thereof having at least 90%, 91%, 92%, 93%, 94%, 95%, 96%, 97%, 98% or 99% identity thereto.

In some embodiments, the antigen binding protein specifically targeting MHC-displayed NTDNNLAVY (SEQ ID NO: 54) comprises a variable heavy chain sequence comprising or consisting of SEQ ID NO: 31 or a variant thereof having at least 96%, 97%, 98% or 99% identity thereto.

In one aspect, the disclosure provides an antigen binding protein specifically binding MHC-displayed NTDNNLAVY (SEQ ID NO: 54), comprising: (a) an antibody heavy chain variable (VH) domain comprising or consisting of the amino acid sequence that is at least about 90%, 91%, 92%, 93%, 94%, 95%, 96%, 97%, 98%, 99% or 100% identical to SEQ ID NO: 31; and/or (b) an antibody light chain variable (VL) domain comprising or consisting of the amino acid sequence that is at least about 90%, 90%, 91%, 92%, 93%, 94%, 95%, 96%, 97%, 98%, 99% or 100% identical to SEQ ID NO: 32.

In some embodiments, the MHC is of HLA supertype A*01, in particular HLA-A*01:01.

In some embodiments, the antigen binding protein is not a T Cell Receptor (TCR).

Off-target peptides expressed in healthy tissues may pose a cross-reactivity risk. In some embodiments, the pMHC binding proteins of the disclosure show a significant reduction in affinity to an HLA-restricted off-target peptide, compared to HLA-restricted NTDNNLAVY (SEQ ID NO: 54). In some embodiments, the antigen binding protein shows low or no binding to MHC-displayed off-target peptides. In some embodiments, an HLA-restricted off-target peptide is derived from the gene product of ICE1, DSG3, PTS, FUS, PTPRC, ROBO1, TBL3, CHTOP, DMXL2, FAP, GPAM, PDE10A, VAV1, ZBTB40, ZNF430, SRP72, PTPN7, ESYT1, WDR81 and/or SLC25A10. In certain embodiments, said peptides are displayed by an HLA of supertype A*01, in particular HLA-A*01:01.

In some embodiments, the antigen binding protein has at least a 50-fold decrease in affinity (e.g., a 75-fold, 80-fold, 90-fold, 100-fold, 120-fold, 150-fold, or 200-fold decrease in affinity) for the MHC-displayed peptides selected from the group consisting of YTDNWLAVY (SEQ ID NO: 57), ETDNNIVVY (SEQ ID NO: 56), NTDNLLTEY (SEQ ID NO: 55), NSDSNLTTY (SEQ ID NO: 61), TADHNLLLY; SEQ ID NO: 62), RTEEALQLY (SEQ ID NO: 71), FLHHTLALY (SEQ ID NO: 72), VTQESKAVY (SEQ ID NO: 73), MSEEHTAVY (SEQ ID NO: 74) and RTDGILALY (SEQ ID NO: 75), relative to affinity to MHC-displayed NTDNNLAVY (SEQ ID NO: 54), as determined by SPR. In some embodiments, the antigen binding protein has at least a 50-fold decrease in affinity (e.g., a 75-fold, 80-fold, 90-fold, 100-fold, 120-fold, 150-fold, or 200-fold decrease in affinity) for the MHC-displayed peptides selected from the group consisting of SEQ ID NO: 55, SEQ ID NO: 56, SEQ ID NO: 57, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74 and SEQ ID NO: 75, relative to affinity to MHC-displayed NTDNNLAVY (SEQ ID NO: 54), as determined by SPR.

As shown in the experimental section, a TBL3 derived peptide (TADHNLLLY; SEQ ID NO: 62) has been identified as having similarity to the KK-LC-1 target peptide NTDNNLAVY (SEQ ID NO: 54) and being highly expressed among many healthy tissues and immune cells. In one aspect, the disclosure provides an antigen binding protein which specifically binds to a major histocompatibility complex (MHC)-displayed NTDNNLAVY (SEQ ID NO: 54), wherein the antigen binding protein has at least about a 10-fold reduction in affinity to MHC-displayed TADHNLLLY (SEQ ID NO: 62), relative to affinity to MHC-displayed NTDNNLAVY (SEQ ID NO: 54). In some embodiments, the antigen binding protein has at least a 50-fold decrease in affinity (e.g., an at least 75-fold, 80-fold, 90-fold, 100-fold, 120-fold, 150-fold, or at least a 200-fold decrease in affinity) for the MHC-displayed peptide TADHNLLLY (SEQ ID NO: 62), relative to MHC-displayed NTDNNLAVY (SEQ ID NO: 54), as determined by SPR.

In certain embodiments, said antigen binding protein binds to MHC-displayed NTDNNLAVY (SEQ ID NO: 54) with an affinity (K_{D}) of about 1 nM to about 30 nM, e.g., at least about 30 nM or lower (i.e. stronger binding), such as at least about 25 nM, 20 nM, 15 nM, 10 nM, 5 nM or at least about 2 nM such as e.g. about 1.8 nM.

In certain embodiments, said antigen binding protein binds to MHC-displayed RTERNVAVY (SEQ ID NO: 66), a GPAM derived off-target peptide, with an affinity (K_{D}) of at least about 200 nM or higher (i.e. corresponding to weaker binding), such as at least about 250 nM, 300 nM, 350 nM, 400 nM, 450 nM, 470 nM, 500 nM, 550 nM, 590 nM, such as about 594 nM. In specific embodiments, the antigen binding protein binds to MHC-displayed RTERNVAVY (SEQ ID NO: 66) with an affinity (K_{D}) of about 450 nM to 600 nM.

In some embodiments, the antigen binding protein is or comprises an antibody, such as a full-length immunoglobulin or an antibody fragment. In some embodiments, the antibody fragment is a Fab, a Fab′, a F(ab′)₂, a scFv, a Fv fragment or a scFab.

In some embodiments, the VH and VL are joined with an amino acid linker. In some embodiments, the amino acid linker comprises the amino acid sequence of SEQ ID NO: 2, SEQ ID NO: 3, SEQ ID NO: 4, SEQ ID NO: 5, SEQ ID NO: 6, or SEQ ID NO: 7, in particular SEQ ID NO: 6.

In certain embodiments, the antigen binding protein is or comprises a scFv having the amino acid sequence of SEQ ID NO: 33, or a variant thereof having at least 90%, 91%, 92%, 93%, 94%, 95%, 96%, 97%, 98% or 99% sequence identity thereto.

In certain embodiments, said scFv has a human germline identity of at least 85%, such as at least about 86% or at least about 87%. For determination, see e.g. Example 13. The higher the human germline identity, the more desirable for therapeutic applications as immunogenicity is reduced.

In some embodiments, the antigen binding protein is chemically or biologically modified, for example glycosylated, PEGylated, PASylated, XTENylated or HESylated.

In some embodiments, the antigen binding protein comprises a light chain and/or a heavy chain comprising an N-terminal and/or C-terminal truncation of 1, 2, 3, 4 or 5 amino acids. In some embodiments, the light chain comprises an N-terminal truncation of 1 or 2 amino acids.

In some embodiments, the antigen binding protein is linked to or combined with a functional entity such as a detectable label, a therapeutic agent or a PK modifying moiety.

In one aspect, the disclosure provides a chimeric antigen receptor (CAR) comprising an antigen binding protein described herein, in particular comprising the scFv described above.

In one aspect, the disclosure provides an immune cell expressing the CAR described herein, in particular a human T cell.

In one aspect, the disclosure provides an antibody drug conjugate (ADC) comprising the antigen binding protein described herein.

In one aspect, the disclosure provides a multispecific antigen binding protein comprising an antigen binding protein described herein, such as a bispecific or trispecific. In some embodiments, the multispecific antigen binding protein comprises: (1) a first antigen binding domain, said first antigen binding domain being the antigen binding protein described herein; and (2) an immune cell binding domain.

Exemplary formats of such multispecific antigen binding protein are or comprise a (scFv)₂, BiTE, BIKE, Dart, diabody, Fab₂, or a Fab-scFv. Said exemplary formats are bispecific and monovalent for each target, e.g., monovalent for CD3 and KK-LC-1, respectively.

In some embodiments, the multispecific antigen binding protein further comprises a second antigen binding domain, such as a second pMHC binding domain. In some embodiments said second antigen binding domain binds to MHC-displayed NTDNNLAVY (SEQ ID NO: 54). In certain embodiments, the antigen binding protein is bispecific. e.g., targeting both CD3 and MHC-displayed NTDNNLAVY (SEQ ID NO: 54); and at the same time is bivalent for MHC-displayed NTDNNLAVY (SEQ ID NO: 54). In some embodiments, said second antigen binding domain is the pMHC antigen binding protein described above. In some embodiments, said second antigen binding domain is identical to the first antigen binding domain.

In some embodiments, any one or more of the first and second antigen binding domain and the immune cell binding domain is or comprises a full-length immunoglobulin or an antibody fragment. In some embodiments, the antibody fragment is or comprises a Fab fragment, a F(ab′)2 fragment, a Fab′ fragment, an Fv fragment, a single chain variable fragment (scFv), and a single domain antibody fragment. In some embodiments, the first antigen binding domain and/or the second antigen binding domain is or comprises a scFv, in particular the scFv of SEQ ID NO: 33 or a variant thereof.

Exemplary embodiments of multispecific antigen binding proteins with a first and a second antigen binding domain as well as an immune cell binding domain include (scFv)₃, tribody, Fab₃, Fab₄, or scFv-Fab-scFv (Fab-scFv₂).

In some embodiments, the immune cell binding domain targets CD3, TCRα, TCRβ, the α/β T Cell Receptor, CD16a, NKG2D, CD94/NKG2C, NKD30, NKD46, CD89, CD64, and CD32a on the surface of an immune cell, in particular a human immune cell.

In some embodiments, the immune cell binding domain is an antibody, more particularly a CD3 binding domain, a CD16a binding domain or BMA031 or a variant thereof.

In certain embodiments, the multispecific antigen binding protein comprises a CD3 binding domain which is derived from any one of antibodies SP34, OKT3 or UCHT1, in particular humanized versions thereof i.e., huSP34, huOKT3 or huUCHT1.

In some embodiments, the CD3 binding domain comprises: (a) an antibody heavy chain variable (VH) domain comprising an HCDR1 amino acid sequence of STYAMN (SEQ ID NO: 38) an HCDR2 amino acid sequence of RIRSKYNNYATYYADSVKG (SEQ ID NO: 39), and an HCDR3 amino acid sequence of HGNFGDSYVSWFAY (SEQ ID NO: 40); and (b) an antibody light chain variable (VL) domain comprising an LCDR1 amino acid sequence of GSSTGAVTTSNYAN (SEQ ID NO: 41), an LCDR2 amino acid sequence of GTNKRAP (SEQ ID NO: 42), and an LCDR3 amino acid sequence of ALWYSNHWV (SEQ ID NO: 43).

In certain embodiments, a CD3 binding domain of the disclosure comprises: (a) an antibody heavy chain variable (VH) domain comprising or consisting of the amino acid sequence of SEQ ID NO: 36 or a variant thereof that is at least about 90%, 91%, 92%, 93%, 94%, 95%, 96%, 97%, 98% or 99% identical thereto; and/or (b) an antibody light chain variable (VL) domain comprising the amino acid sequence of SEQ ID NO: 37 or a variant thereof that is at least about 90%, 91%, 92%, 93%, 94%, 95%, 96%, 97%, 98% or 99% identical thereto.

In some embodiments, the CD3 binding domain is or comprises a Fab fragment, wherein said Fab fragment comprises: (1) a heavy chain (HC) comprising a CH1 domain and said VH; and (2) a light chain (LC) comprising a CL domain and said VL. For example, in certain embodiments, said Fab fragment comprises or consists of (1) the HC of SEQ ID NO: 34, or variant thereof having at least 90%, 91%, 92%, 93%, 94%, 95%, 96%, 97%, 98% or 99% sequence identity thereto; and (2) the LC of SEQ ID NO: 35, or variant thereof having at least 90%, 91%, 92%, 93%, 94%, 95%, 96%, 97%, 98% or 99% sequence identity thereto. In some embodiments, the CH1 domain comprises at least 5 amino acids of an antibody hinge region, in particular the amino acid sequence EPKSC (SEQ ID NO: 17).

In some embodiments, the first antigen binding domain is operably linked to the C-terminus of the heavy chain or the N-terminus of the heavy chain of the Fab fragment. In some embodiments, the second antigen binding domain is operably linked to the C-terminus of the light chain or the N-terminus of the light chain of the Fab fragment.

In some embodiments: a) the first antigen binding domain is or comprises an scFv being linked to the C-terminus of the Fab domain heavy chain and the second antigen binding domain comprises an scFv being linked to the C-terminus of the Fab domain light chain; b) the first antigen binding domain is or comprises an scFv being linked to the N-terminus of the Fab domain heavy chain and the second antigen binding domain comprises an scFv being linked to the N-terminus of the Fab domain light chain; c) the first antigen binding domain is or comprises an scFv being linked to the N-terminus of the Fab domain heavy chain and the second antigen binding domain comprises an scFv being linked to the C-terminus of the Fab domain light chain; or d) the first antigen binding domain is or comprises an scFv being linked to the C-terminus of the Fab domain heavy chain and the second antigen binding domain comprises an scFv being linked to the N-terminus of the Fab domain light chain.

In some embodiments, the scFv is linked to the Fab domain with an amino acid linker. In some embodiments, the amino acid linker comprises (GGGGS)ₙ (SEQ ID NO: 1), wherein n is an integer between 1 and 5, in particular n being 1, i.e., GGGGS (SEQ ID NO: 2). In certain embodiments, one scFv is bound to the CH1 domain of the Fab via EPKSCGGGGS (SEQ ID NO: 18) and a second scFv is bound to the CL domain via GGGGS (SEQ ID NO: 2).

In some embodiments, the multispecific antigen binding protein does not comprise an Fc domain.

In certain embodiments, the bispecific CD3×KK-LC-1 binding protein is a Fab-scFv comprising: (1) a HC comprising or consisting of an amino acid sequence that is at least about 90%, 91%, 92%, 93%, 94%, 95%, 96%, 97%, 98%, 99% or 100% identical to the amino acid sequence of SEQ ID NO: 52; and a LC comprising of an amino acid sequence that is at least about 90%, 91%, 92%, 93%, 94%, 95%, 96%, 97%, 98%, 99% or 100% identical to the amino acid sequence of SEQ ID NO: 35.

In certain embodiments, the bispecific CD3×KK-LC-1 binding protein is a Fab-scFv comprising the scFv of SEQ ID NO: 33, being linked to the C-terminus of the HC of SEQ ID NO: 34 and a LC of SEQ ID NO: 35, or variants of said sequences that are at least about 90%, 95%, 96%, 97%, 98% or 99% identical thereto.

In some embodiments, the multispecific antigen binding protein is a bispecific Fab-scFv₂ comprising: a HC comprising or consisting of an amino acid sequence that is at least about 90%, 91%, 92%, 93%, 94%, 95%, 96%, 97%, 98%, 99% or 100% identical to the amino acid sequence of SEQ ID NO: 52; and a LC comprising or consisting of an amino acid sequence that is at least about 90%, 91%, 92%, 93%, 94%, 95%, 96%, 97%, 98%, 99% or 100% identical to the amino acid sequence of SEQ ID NO: 53. Said bispecific Fab-scFv₂ is thus a humanized, affinity-matured, and stability-engineered T cell engager.

In some embodiments, the light chain and/or heavy chain comprises an N-terminal and/or C-terminal truncation of 1, 2, 3, 4, or 5 amino acids. In some embodiments, the light chain comprises an N-terminal truncation of 1 or 2 amino acids.

In some embodiments, the multispecific antigen binding protein comprises a pyroglutamate (pE) at position 1 instead of glutamine (Q) or glutamate (E) of the light chain and/or heavy chain. In some embodiments, the multispecific antigen binding protein comprises a pyroglutamate (pE) at position 1 instead of glutamine (Q) of the light chain.

In one aspect, the disclosure provides a nucleic acid encoding the antigen binding protein described herein such as the multispecific antigen binding protein or the CAR described herein, in particular an isolated nucleic acid.

In one aspect, the disclosure provides a vector comprising the nucleic acid described herein, in particular an expression vector or a viral vector.

In one aspect, the disclosure provides a host cell population comprising the vector or the nucleic acid described herein. In certain embodiments, said host cells are HEK or CHO cells.

In one aspect, the disclosure provides a method of manufacturing the antigen binding protein described herein (e.g. the multispecific antigen binding protein) or the ADC described herein, comprising the steps of: (i) cultivating the host cell population described above under conditions allowing expression of the antigen binding protein (e.g, the multispecific); followed by (ii) recovering the antigen binding protein; and optionally (iii) further purifying and/or modifying and/or formulating the antigen binding protein.

In some embodiments, the antigen binding protein described herein, in particular the monovalent antibody, is for use in diagnostics.

In one aspect, the disclosure provides a kit comprising the antigen binding protein described herein, e.g., the multispecific antigen binding protein described herein.

In one aspect, the disclosure provides the antigen binding protein described herein, the CAR described herein, the immune cell described herein, the ADC described herein, the multispecific antigen binding protein described herein, the nucleic acid described herein, the vector described herein, or the host cell population described herein, for use in a method for inhibiting growth or proliferation of cancer cells.

In one aspect, the disclosure provides the antigen binding protein described herein, the CAR described herein, the immune cell described herein, the ADC described herein, the multispecific antigen binding protein described herein, the nucleic acid described herein, the vector described herein, or the host cell population described herein, for use in a method of redirecting a T cell to a KK-LC-1-expressing cancer cell.

In one aspect, the disclosure provides the antigen binding protein described herein, the CAR described herein, the immune cell described herein, the ADC described herein, the multispecific antigen binding protein described herein, the nucleic acid described herein, the vector described herein, or the host cell population described herein, for use in the treatment of cancer, wherein the cancer is a solid tumor and/or a hematological tumor, optionally wherein the cancer is selected from cervical cancer, pancreatic cancer, esophageal cancer, gastric cancer (such as gastric adenocarcinoma), lung cancer (such as adenocarcinoma, lung squamous cancer or Non-small cell lung cancer (NSCLC) (e.g., non-squamous NSCLC)), or breast cancer (such as triple-negative breast cancer).

In one aspect, the disclosure provides a pharmaceutical composition comprising the antigen binding protein described herein, the CAR described herein, the immune cell described herein, the ADC described herein, the multispecific antigen binding protein described herein, the nucleic acid described herein, or the vector described herein, and a pharmaceutically acceptable buffer.

In one aspect, the disclosure provides for the use of the antigen binding protein described herein, the CAR described herein, the immune cell described herein, the ADC described herein, the multispecific antigen binding protein described herein, the nucleic acid described herein, the vector described herein, the host cell population described herein, or the pharmaceutical composition described herein, in the manufacture of a medicament.

In one aspect, the disclosure provides the antigen binding protein described herein, the CAR described herein, the immune cell described herein, the ADC described herein, the multispecific antigen binding protein described herein, the nucleic acid described herein, the vector described herein, the host cell population described herein, or the pharmaceutical composition described herein, for use as medicament.

In one aspect, the disclosure provides for the use of the antigen binding protein described herein, the CAR described herein, the immune cell described herein, the ADC described herein, the multispecific antigen binding protein described herein, the nucleic acid described herein, the vector described herein, or the pharmaceutical composition described herein, the host cell population described herein, in the treatment of a disease, in particular cancer.

In one aspect, the disclosure provides a method of treating a cancer expressing MHC-displayed NTDNNLAVY (SEQ ID NO: 54) in a patient in need thereof comprising administering to the patient a therapeutically effective amount of the antigen binding protein described herein, the CAR described herein, the immune cell described herein, the ADC described herein, the multispecific antigen binding protein described herein, the nucleic acid described herein, the vector described herein, the host cell population described herein, or the pharmaceutical composition described herein.

In some embodiments, the cancer is a solid tumor and/or a hematological tumor, optionally wherein the cancer is selected from cervical cancer, pancreatic cancer, esophageal cancer, gastric cancer such as gastric adenocarcinoma, lung cancer (such as adenocarcinoma, lung squamous cancer or Non-small cell lung cancer (NSCLC) (e.g., non-squamous NSCLC), or breast cancer such as triple-negative breast cancer.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 depicts HLA-A*01:01/KK-LC-1 identification in non-small cell lung cancer (NSCLC) biopsies. Target quantification was performed by immunoprecipitation followed by mass spectrometry analysis of the tumor and normal adjacent tissue (NAT).

FIG. 2 depicts expression levels of off-target peptides with sequence similarity to the KK-LC-1 target peptide in healthy tissues, as determined by immunopeptidomic analysis. Numbers refer to normalized intensity score: a value of 1000 refers to 0.1% of the total peptide signal intensity from the entire sample.

FIG. 3 depicts expression levels of off-target peptides with sequence similarity to the KK-LC-1 target peptide in healthy immune cells, as determined by immunopeptidomic analysis. Numbers refer to normalized intensity score: a value of 1000 refers to 0.1% of the total peptide signal intensity from the entire sample.

FIG. 4 depicts interferon gamma (IFNγ) release as a measure of T cell activation upon treatment with M2883 of antigen presenting T2A1 cells. T2A1 cells were loaded with KK-LC-1 target peptide and physiologically relevant off-target peptides displaying high sequence identity to KK-LC-1 and confirmed presentation in healthy tissues. ULOQ—upper limit of quantification.

FIG. 5 depicts Granzyme B (GrzB) release upon treatment of antigen presenting T2A1 cells with M2883 in the presence of PBMCs. T2A1 cells were loaded with KK-LC-1 target peptide and mutated variants thereof with alanine, arginine and aspartate substitutions at each amino acid position. Alanine at position seven and aspartate at position three were substituted with a serine.

FIG. 6 depicts in vitro cytotoxicity of M2883 in KK-LC-1-positive HLA-A*01:01-positive cancer cells NCI-H1703 and EKVX and KK-LC-1-negative HLA-A*01:01-positive cancer cells SK-MEL-30 and PC-3. CpC—copies per cell.

FIGS. 7A-7B depict in vitro safety of M2883 in (FIG. 7A) HLA-A*01:01-positive primary cells HCMEC_147 (Human Cardiac Microvascular Endothelial Cells), HBSMC_297 (Human Bronchial Smooth Muscle Cells), HSAEpC_645 (Human Small Airway Epithelial Cells) and HPMEC_770 (Human Pulmonary Microvascular Endothelial Cells), and (FIG. 7B) human cardiac fibroblasts (HCF_722), normal human lung fibroblasts (NHLF_19232) and human aortic smooth muscle cells (HAoSMC_173). Human pulmonary fibroblasts (HPF_646) served as an HLA-A*01:01-negative, KK-LC-1-negative control. HLA-A*01:01-positive and KK-LC-1-positive cancer cell line NCI-H1703 served as a positive control. ULOQ—upper limit of quantification.

FIG. 8 depicts in vitro safety of M2883 and M3903 in HLA-A*01:01-positive primary human lung fibroblasts (NHLF_19232) and human aortic smooth muscle cells (HAoSMC_173). HLA-A*01:01-positive, KK-LC-1-positive control cancer cell line NCI-H1703 served as a positive control. ULOQ—upper limit of quantification.

FIG. 9 depicts the quality of M3904 drug substance produced using a stable CHO pool and 14-day fed-batch process at a 10 L scale. Protein purity was assessed by size-exclusion chromatography (SEC), cation-exchange chromatography (CEX) and hydrophobic interaction chromatography (HIC). MP—main peak.

FIG. 10 depicts in vitro cytotoxicity of M3904 in KK-LC-1-positive HLA-A*01:01-positive cancer cells NCI-H1703 and EKVX and KK-LC-1-negative HLA-A*01:01-positive cancer cells SK-MEL-30 and PC-3.

FIG. 11 depicts the release of IFNγ, IL-2, IL-6, TNFα, Granzyme B and IL-10 following treatment with M3904. The HLA-A*01:01-positive and KK-LC-1-positive cancer cell line NCI-H1703 was incubated with PBMCs and varying concentrations of M3904. Cytokine levels were quantified in the culture supernatants.

FIG. 12 depicts T cell activation following treatment with M3904. The HLA-A*01:01-positive and KK-LC-1-positive cancer cell line NCI-H1703 was incubated with PBMCs and varying concentrations of M3904. T cell activation markers CD69, CD25 and T cell proliferation marker Ki67 were quantified on CD3+, CD4+ and CD8+ T cells.

FIG. 13 depicts in vitro safety of M3904 in HLA-A*01:01-positive human primary cells. HLA-A*01:01-positive, KK-LC-1-positive control cancer cell line NCI-H1703 served as a positive control.

FIGS. 14A-14B depict a molecular binding profile of M3904. FIG. 14A depicts a heat map representing the binding of M3903, a monovalent variant of M3904, to a series of peptides, each containing a single amino acid substitution, as determined by SPR. The x-axis represents the position of the substituted amino acid within the peptide sequence, while the y-axis denotes the specific amino acid residue introduced at each position. Color intensity corresponds to binding affinity, which was normalized to the native KK-LC-1 peptide affinity value, with a gradient from white (no binding) to black (200% and higher binding than to KK-LC-1), as indicated by the accompanying color scale. FIG. 14B depicts a sequence logo generated based on the X-scan analysis representing the binding preferences of M3903 and M3904. The height of each letter at a given position corresponds to the relative binding contribution of that amino acid, with larger letters indicating stronger binding preferences. The x-axis represents the position of the amino acid within the peptide sequence, while the y-axis denotes the information content, reflecting sequence conservation and binding specificity.

FIG. 15 depicts Granzyme B (GrzB) release upon treatment of antigen presenting T2A1 cells with M3904 in the presence of PBMCs. T2A1 cells were loaded with KK-LC-1 target peptide as positive control and potential off-target peptides posing cross-reactivity risk.

FIGS. 16A-16C depict the developability properties of M3904. Solubility (FIG. 16A), shelf-life stability (FIG. 16B) and stability in physiological conditions (FIG. 16C) are shown. Size exclusion chromatography was used to determine the purity of M3904 at the tested conditions. The peak maximum in the overlays was normalized to the same signal height. Chromatograms are displayed with 14% time offset and 5% signal offset.

FIGS. 17A-17B depict the modular architecture of the Fab-scFv and the Fab-scFv₂ format. FIG. 17A: a Fab-scFv scaffold, an exemplary embodiment of a bispecific antigen binding protein being monovalent for binding to MHC-displayed NTDNNLAVY (SEQ ID NO: 54) and monovalent for T cell binding. Here, a single scFv binding to MHC-displayed NTDNNLAVY (SEQ ID NO: 54) is attached via a linker to the C-terminal of the Fab heavy chain. FIG. 17B: a Fab-(scFv)₂ scaffold, an exemplary embodiment of a bispecific antigen binding protein as described herein, being bivalent for binding to MHC-displayed NTDNNLAVY (SEQ ID NO: 54) and monovalent for T cell binding. The Fab is directed against a T cell antigen, e.g., CD3. A first scFv binding to MHC-displayed NTDNNLAVY (SEQ ID NO: 54) is attached via a linker to the C-terminal of the Fab heavy chain (CH1) and a second scFv is attached via a linker to the C-terminal of the Fab light chain (CL domain).

### DETAILED DESCRIPTION

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein is well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of the term “including,” as well as other forms, such as “includes” and “included,” is not limiting.

So that the invention may be more readily understood, certain terms are first defined.

As used herein, the term “Kita-Kyushu lung cancer antigen-1” or “KK-LC-1” or “CT83” or “Cxorf61” refers to a protein belonging to the cancer/testis antigen (CTA) protein family. CTAs are a class of cancer antigens that are mainly expressed in tumors and testicular tissues but display low to no expression in other normal tissues. The antigen binding proteins of the disclosure bind the MHC-displayed NTDNNLAVY (SEQ ID NO: 54) peptide, said peptide being derived from KK-LC-1 protein (amino acid 52-60) encoded by the CT83 gene, in particular when presented by an HLA-A*01:01 (SEQ ID NO: 23) molecule.

“CD3”, the cluster of differentiation 3 co-receptor (or co-receptor complex) of the T cell receptor, is a complex composed of four distinct chains. In mammals, the complex contains a CD3γ (gamma) chain/subunit, a CD3δ (delta) chain/subunit, and two CD3ε (epsilon) chains/subunits. Reference to CD3 as the cell surface protein of an immune cell is made herein throughout. The term “CD3” refers to any native CD3 from any vertebrate source, including primates. In certain embodiments, the antigen binding proteins of the disclosure specifically bind to human CD3, in particular to the CD3ε (epsilon) chain/subunit of CD3 (see e.g., UniProt (www.uniprot.org) accession no. P07766 (version 189), or NCBI (www.ncbi.nlm.nih.gov/) RefSeq NP_000724.1.) The CD3 molecule may be the full-length, unprocessed CD3 molecule or a fragment or variant thereof that e.g., results from processing in the cell. For example, such variants may be naturally occurring variants like splice variants or allelic variants. In certain embodiments, the antigen binding proteins disclosed herein bind to an epitope of CD3 that is conserved among the CD3 antigens from different species, such as non-human primates (e.g., cynomolgus monkeys) or rodents (e.g., mice, rats). In certain embodiments, the antigen binding proteins are not cross-reactive with a CD3 antigen from rodents (e.g., mouse or rat) or minipigs.

As used herein, the term “antigen binding protein” refers to a protein that specifically binds to or is immunologically reactive with an antigen or epitope. The term “antigen binding protein” includes “antibody” or more particularly “antibody fragment”, but also non-immunoglobulin-based binding proteins. Non-limiting examples of non-immunoglobulin-based binding proteins include DARPins, affimers, monobodies, anticalins, fynomers, and affibodies. Non-immunoglobulin-based binding proteins are described in further detail in Simeon et al. (Protein Cell. 2018. 9 (1): 3-14) and Olaleye et al. (Biomolecules. 2021. 11 (12): 1791), each of which is incorporated herein by reference.

The term “antibody” refers to an immunoglobulin molecule or immunoglobulin derived molecule that specifically binds to, or is immunologically reactive with an antigen or epitope, and includes both polyclonal and monoclonal antibodies, as well as functional antibody fragments, including but not limited to fragment antigen-binding (Fab) fragments, F(ab′)₂ fragments, Fv fragments, recombinant IgG (rIgG) fragments, single chain variable fragments (scFv) and single domain antibodies (e.g., sdAb, sdFv, nanobody, VHH fragments), as long as they exhibit the desired antigen-binding activity. The antibody may thus be a single domain antibody or comprise at least one variable light and at least one variable heavy chain. In one embodiment, the at least one variable light and at least one variable heavy chain are displayed as a single polypeptide chain. In certain embodiments, the term “antibody” or “antigen binding protein” includes germline derived antibodies. The term “antibody” or “antigen binding protein” includes genetically engineered or otherwise modified forms of immunoglobulins, such as intrabodies, peptibodies, chimeric antibodies, fully human antibodies, humanized antibodies, heteroconjugate antibodies (e.g., bispecific antibodies, diabodies, triabodies, tetrabodies, tandem di-scFv, tandem tri-scFv) and the like. Unless otherwise stated, the term “antibody” should be understood to encompass functional antibody fragments thereof.

In certain embodiments, the antigen binding protein is not a T cell receptor (TCR), including but not limited to, a soluble TCR.

In certain embodiments, the antigen binding protein is multispecific (i.e., binds to two or more different target molecules or to two or more epitopes on the same target molecule). In certain embodiments, the antigen binding protein is bispecific and e.g., binds to two different target molecules or to two different epitopes on the same target molecule. In certain embodiments, the antigen binding protein is trispecific and e.g., binds to at least three different target molecules. In certain embodiments thereof, a trispecific antigen binding protein comprises three binding sites and binds to antigens on at least two distinct cells.

The antigen binding protein may be monovalent or multivalent, i.e., having one or more antigen binding sites. Non-limiting examples of monovalent antigen binding proteins include scFv, Fab, scFab, dAb, VHH, V (NAR), DARPins, affilins and nanobodies. A multivalent antigen binding protein can have two, three, four or more antigen binding sites; accordingly, the antigen binding protein is “bivalent”, “trivalent”, “tetravalent” and so forth. Non-limiting examples of multivalent antigen binding proteins include full-length immunoglobulins, F(ab′)₂ fragments, bis-scFv (or tandem scFv or BiTE), DART, diabodies, scDb, DVD-Ig, IgG-scFab, scFab-Fc-scFab, IgG-scFv, scFv-Fc, scFv-fc-scFv, Fv₂-Fc, FynomABs, quadroma, CrossMab, DuoBody, triabodies and tetrabodies. In some embodiments, the multivalent antigen binding protein is bivalent, i.e., two binding sites are present. In some embodiments, the multivalent antigen binding protein is bispecific, i.e., the antigen binding protein is directed against two different targets or two different target sites on one target molecule. In some embodiments, the multivalent antigen binding protein includes more than two, e.g., three or four different binding sites for three or four, respectively, different antigens. Such antigen binding protein is multivalent and multispecific, in particular tri- or tetra-specific, respectively.

In some embodiments, the antigen binding proteins are multispecific (e.g., bispecific), such as, without being limited to, diabodies, single-chain diabodies, DARTs, BiTEs, BIKEs, tandem scFvs or IgG-like asymmetric heterobispecific antibodies. In certain embodiments, one of the binding specificities of the multispecific antigen binding protein is an immune cell engager (i.e., comprising affinity to a cell surface protein of an immune cell, also referred to as “immune cell binding domain”). Examples of immune cells that may be recruited include, but are not limited to, T cells, B cells, natural killer (NK) cells, natural killer T (NKT) cells, neutrophil cells, monocytes, and macrophages. Examples of surface proteins that may be used to recruit immune cells includes, without being limited to, CD3, TCRα, TCRβ, CD16, NKG2D, CD94/NKG2C, NKD30, NKD46, CD89, CD64, and CD32. In certain embodiments, the immune cell target antigen is CD3. In certain embodiments, the multispecific antigen binding protein is a bispecific targeting CD3 and MHC-displayed NTDNNLAVY (SEQ ID NO: 54). In certain embodiments, said bispecific antigen binding protein is monovalent for CD3 and bivalent for MHC-displayed NTDNNLAVY (SEQ ID NO: 54).

As used herein, the term “monoclonal antibody” refers to an antibody obtained from a population of substantially homogeneous antibodies, i.e., antibodies that bind to the same epitope and/or are identical in sequence. A population of polyclonal antibodies, in contrast, will bind to multiple epitopes and comprises antibodies of different sequences. A monoclonal antibody preparation may or may not comprise to a minor extent variant antibodies, e.g., due to naturally occurring mutations. Such variants may e.g., be generated through posttranslational modifications, such as clipping at the N-terminal or C-terminal end of the light and/or heavy chain, or pyroglutamate formation on the N terminus of the polypeptide chain (see e.g., Liu Y D, et al. J Biol Chem. 2011 Apr. 1; 286 (13): 11211-7). Depending on the methods and antibody used, the percentage of variants in the mixture varies, and may involve substantially all antibodies produced, or a very low percentage.

As used herein, a “single-chain variable fragment” (scFv) is an antigen binding protein comprising a heavy chain variable domain (VH) linked to a light chain variable domain (VL). The VH and VL domains of the scFv are linked via any appropriate art recognized linker. Such linkers include, but are not limited to, repeated GGGGS (SEQ ID NO: 2) amino acid sequences or variants thereof. The scFv is generally free of antibody constant domain regions, although an scFv of the disclosure may be linked or attached to antibody constant domain regions (e.g., antibody Fc domain) to alter various properties of the scFv, including, but not limited to, increased serum or tissue half-life. An scFv generally has a molecular weight of about 25 kDa and a hydrodynamic radius of about 2.5 nm.

As used herein, a “Fab” (Fab=fragment antigen-binding) is a portion of an immunoglobulin that retains the antigen-binding regions of the original antibody while lacking the Fc portion (Fc=fragment crystallizable region). In an experimental setting, Fc and Fab fragments can be generated through papain digestion of a full-length antibody, resulting in two monovalent Fab fragments and one Fc portion. The Fab fragments, with a molecular weight of approximately 50 kDa, have better permeability characteristics than full-length antibodies. With the Fc portion being absent, Fab fragments do not interact with Fc receptors on cells or trigger any other in vivo Fc-effector function. Fabs are composed of one heavy and one light chain, each chain having a constant (HC or LC) and a variable domain (VH or VL). “Fab”, “Fab fragment” or “Fab domain” as used interchangeably herein is an antibody fragment comprising a light chain fragment comprising a variable light (VL) domain and a constant domain of the light chain (CL), and variable heavy (VH) domain and a first constant domain (CH1) of the heavy chain. A F(ab′)₂ comprises two antigen-binding regions joined at the hinge through disulfides.

As used herein, a “VHH”, “nanobody”, “heavy-chain only antibody”, “single domain antibody”, or “sdAb” is an antigen binding protein comprising a single heavy chain variable domain derived from the species of the Camelidae family, which includes camels, llama, alpaca. A VHH generally has a molecular weight of about 15 kDa.

The antigen binding proteins of the disclosure may comprise one or more linkers for linking the domains of the antigen binding protein (e.g., linking a VH and VL to form a scFv, or linking multiple binding domains to form a multispecific antigen binding protein).

Illustrative examples of linkers include glycine polymers (Gly)ₙ; glycine-serine polymers (GlynSer)ₙ, where n is an integer of at least one, two, three, four, five, six, seven, or eight; glycine-alanine polymers; alanine-serine polymers; and other flexible linkers known in the art.

Glycine and glycine-serine polymers are relatively unstructured and therefore may be able to serve as a neutral tether between domains of fusion proteins such as the antigen binding proteins described herein. Glycine accesses significantly more phi-psi space than other small side chain amino acids and is much less restricted than residues with longer side chains (Scheraga, Rev. Computational Chem. 1:1173-142 (1992)). A person skilled in the art will recognize that design of an antigen binding protein in particular embodiments can include linkers that are all or partially flexible, such that the linker can include flexible linker stretches as well as one or more stretches that confer less flexibility to provide a desired structure.

Linker sequences can however be chosen to resemble natural linker sequences, for example, using the amino acid stretches corresponding to the beginning of human CH1 and Ck sequences or amino acid stretches corresponding to the lower portion of the hinge region of human IgG.

The design of the peptide linkers connecting VL and VH domains in the scFv moieties are flexible linkers generally composed of small, non-polar or polar residues such as, e.g., Gly, Ser and Thr. A particularly exemplary linker connecting the variable domains of the scFv moieties is the (Gly₄Ser)₄ linker (SEQ ID NO: 4), where 4 is the exemplary number of repeats of the motif.

Linkers connecting the scFv antigen binding proteins to the Fab domain are also envisioned. In certain embodiments, the scFv antigen binding proteins are linked to the CH1 and CL domains of the Fab with a Gly-Ser linker. In certain embodiments, the linker comprises the amino acid sequence GGGGS (SEQ ID NO: 2). In certain embodiments, the linker comprises 3-5 repetitions of the GGGGS (SEQ ID NO: 2) motif. In certain embodiments, the amino acid linker comprises the amino acid sequence GGGGSGGGGSGGGGS (SEQ ID NO: 4), GGGGSGGGGSGGGGSGGGGS (SEQ ID NO: 5), or GGGGSGGGGSGGGGSGGGGAS (SEQ ID NO: 7). Alternatively, linker sequences connecting the variable domains may include glycine polymers (G) n; glycine-alanine polymers; alanine-serine polymers; and other flexible linkers known in the art.

Other exemplary linkers include, but are not limited to the following amino acid sequences: GGG; DGGGS (SEQ ID NO: 8); TGEKP (SEQ ID NO: 9) (Liu et al, Proc. Natl. Acad. Sci. 94:5525-5530 (1997)); GGRR (SEQ ID NO: 10); (GGGGS) n (SEQ ID NO: 1) wherein n=1, 2, 3, 4 or 5 (Kim et al, Proc. Natl. Acad. Sci. 93:1156-1160 (1996)); EGKSSGSGSESKVD (SEQ ID NO:11) (Chaudhary et al., Proc. Natl. Acad. Sci. 87:1066-1070 (1990)); KESGSVSSEQLAQFRSLD (SEQ ID NO: 12) (Bird et al., Science 242:423-426 (1988)), GGRRGGGS (SEQ ID NO: 13); LRQRDGERP (SEQ ID NO: 14); LRQKDGGGSERP (SEQ ID NO: 15); and GSTSGSGKPGSGEGSTKG (SEQ ID NO: 16) (Cooper et al, Blood, 101 (4): 1637-1644 (2003)). Alternatively, flexible linkers can be rationally designed using a computer program capable of modeling the 3D structure of proteins and peptides or by phage display methods.

The antibodies may comprise a variable light (VL) domain and a variable heavy (VH) domain. Each VL and VH domain further comprises a set of three CDRs.

As used herein, the term “complementarity determining region” or “CDR” refers to non-contiguous sequences of amino acids within antibody variable regions, which confer antigen specificity and affinity. In general, there are three CDRs in each heavy chain variable domain (HCDR1, HCDR2, HCDR3 or CDRH1, CDRH2, CDRH3, as used synonymously herein) and three CDRs in each light chain variable domain (LCDR1, LCDR2, LCDR3 or CDRL1, CDRL2, CDRL3, as used synonymously herein). “Framework regions” or “FRs” are known in the art to refer to the non-CDR portions of the variable domains of the heavy and light chains. In general, there are four FRs in each heavy chain variable domain (HFR1, HFR2, HFR3, and HFR4), and four FRs in each light chain variable domain (LFR1, LFR2, LFR3, and LFR4). Accordingly, an antibody variable region amino acid sequence can be represented by the formula FR1-CDR1-FR2-CDR2-FR3-CDR3-FR4. Each segment of the formula, i.e., FR1, CDR1, FR2, CDR2, FR3, CDR3, and FR4, represents a discrete amino acid sequence (or a polynucleotide sequence encoding the same) that can be mutated, including one or more amino acid substitutions, deletions, and insertions. In certain embodiments, an antibody variable light chain amino acid sequence can be represented by the formula LFR1-LCDR1-LFR2-LCDR2-LFR3-LCDR3-LFR4. In certain embodiments, an antibody variable heavy chain amino acid sequence can be represented by the formula HFR1-HCDR1-HFR2-HCDR2-HFR3-HCDR3-HFR4.

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme).

The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Table 1, below, lists exemplary position boundaries of LCDR1, LCDR2, LCDR3 and HCDR1, HCDR2, HCDR3 of an antibody, as identified by Kabat, Chothia, and Contact schemes, respectively. For CDRH1, residue numbering is listed using both the Kabat and Chothia numbering schemes. CDRs are located between FRs, for example, with CDRL1 located between LFR1 and LFR2, and so forth. It is noted that because the shown Kabat numbering scheme places insertions at H35A and H35B, the end of the Chothia CDRH1 loop when numbered using the shown Kabat numbering convention varies between H32 and H34, depending on the length of the loop.

TABLE 1
Exemplary Position Boundaries of CDRs

| CDR | Kabat | Chothia | Contact |
|---|---|---|---|
| LCDR1 | L24-L34 | L24-L34 | L30-L36 |
| LCDR2 | L50-L56 | L50-L56 | L46-L55 |
| LCDR3 | L89-L97 | L89-L97 | L89-L96 |
| HCDR1 | H31-H35B | H26-H32 . . . 34 | H30-H35B |
| (Kabat Numbering¹) | | | |
| HCDR1 | H31-H35 | H26-H32 | H30-H35 |
| (Chothia Numbering²) | | | |
| HCDR2 | H50-H65 | H52-H56 | H47-H58 |
| HCDR3 | H95-H102 | H95-H102 | H93-H101 |

¹Kabat et al. (1991), “Sequences of Proteins of Immunological Interest,” 5^{th} Ed. Public Health Service, National Institutes of Health, Bethesda, MD
²Al-Lazikani et al. (1997), J. Mol. Biol. 273: 927-948

Thus, unless otherwise specified, a “CDR” or “complementary determining region,” or individual specified CDRs (e.g., CDRH1, CDRH2), of a given antibody or fragment thereof, such as a variable domain thereof, should be understood to encompass a (or the specific) complementary determining region as defined by Kabat. However, CDRH1 as used herein is defined by residues H30-H35B (Kabat numbering) or H30-H35 (Chothia numbering). Likewise, unless otherwise specified, an “FR” or “framework region,” or individual specified FRs (e.g., “HFR1,” “HFR2”) of a given antibody or fragment thereof, such as a variable domain thereof, should be understood to encompass a (or the specific) framework region as defined by Kabat. In some instances, the particular amino acid sequence of a CDR or FR is given.

In certain embodiments, the antigen binding proteins disclosed here are rabbit-derived antigen binding proteins. In certain embodiments, the rabbit antigen binding proteins are humanized. As used herein, the term “humanized” or “humanization” refers to an antigen binding protein that has been altered to make it more like a human antibody. Non-human antigen binding proteins, such as rabbit antigen binding proteins, would elicit a negative immune reaction if administered to a human for therapy. It is therefore advantageous to humanize the rabbit antigen binding proteins for later therapeutic use.

In certain embodiments, the antigen binding proteins are humanized through resurfacing (i.e., remodel the solvent-accessible residues of the non-human framework such that they become more human-like). Resurfacing strategies are described in more detail in WO2004/016740, WO2008/144757, and WO2005/016950, each of which is incorporated herein by reference.

In certain embodiments, the antigen binding proteins are humanized through CDR grafting (i.e., inserting the rabbit antigen binding protein CDRs into a human antibody acceptor framework). Grafting strategies and human acceptor frameworks are described in more detail in WO2009/155726, incorporated herein by reference.

As used herein, “sequence identity” between two polypeptides is determined by comparing the amino acid sequence of one polypeptide to the sequence of a second polypeptide. Similarly, “sequence identity” between two polynucleotides is determined by comparing the nucleotide sequence of one polynucleotide to the sequence of a second polynucleotide. The terms “% identical”, “% identity” or similar terms are intended to refer, in particular, to the percentage of nucleotides or amino acids (as applicable) which are identical after the sequences to be compared have been aligned to yield maximum identity, potentially introducing gaps. Said percentage may be purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. For example, the optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using the algorithms (GAP, BESTFIT, FASTA, BLAST P, BLAST N and TFASTA in Wisconsin Genetics Software Package, Genetics Computer Group, 575 Science Drive, Madison, Wis., or Clustal Omega). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq &LINK_LOC-align2seq). Typically, the % identity is determined over the entire length of the query sequence on which the analysis is performed.

A variant polypeptide, such as an antigen binding protein, may contain one or more substitutions (e.g., conservative substitutions), insertions, or deletions relative to the reference sequence. In certain embodiments, the variant polypeptide comprises one, two or three substitutions, insertions and/or deletions relative to the reference sequence. Substitutions, insertions, or deletions may result in the change of one or more biophysical parameters but particularly preferred are tolerated changes so that the polypeptide retains the desired activity. In some embodiments, the variant polypeptide comprises one or more tolerated substitutions, such as conservative substitutions. In certain embodiments, such variant polypeptide maintains physically, biologically, chemically and/or functionally the properties of the corresponding reference sequence.

“Specifically recognizes” or “specifically binds” refers to the ability of the antigen binding proteins to bind selectively to the antigen in contrast to non-specific interactions with unrelated proteins which do not comprise the binding epitope. Suitable assays for determining the specific binding are described below.

As used herein, the term “affinity” (or “binding affinity” as used interchangeably herein) refers to the strength of the interaction between an antibody's antigen binding site and the epitope to which it binds. As readily understood by those skilled in the art, an antibody or antigen binding protein affinity may be reported as an equilibrium dissociation constant (K_{D}) in molarity (M). The equilibrium dissociation constant K_{D} is calculated from the association rate constant kₐ (having the unit M⁻¹s⁻¹) and the dissociation rate constant k_{d} (having the unit s⁻¹) by k_{d}/kₐ. The antibodies of the disclosure may have K_{D} values in the range of 10⁻⁸ to 10⁻¹⁴ M to the target peptide.

The ability of an antibody to bind to a specific antigenic determinant (e.g., a target peptide-MHC) can be measured either through an enzyme-linked immunosorbent assay (ELISA) or other techniques familiar to one of skill in the art, e.g., surface plasmon resonance (SPR) technique (conducted e.g. using a BIAcore instrument) (Liljeblad et al., Glyco J 17, 323-329 (2000)), and traditional binding assays (Heeley, Endocr Res 28, 217-229 (2002)). Generally, kinetic rate constants can be determined at temperatures in the range of 15° C. to 37° C. The present specification makes reference to kinetic rate constants determined by SPR throughout. Typically, in embodiments pertaining to each reference to SPR throughout the present specification, association rate constant values, dissociation rate constant values and equilibrium dissociation constant values recited herein are determined by SPR at 25° C. SPR based on the monovalent antigen binding protein. Preferably, the SPR-based system used is a Biacore SPR system. The skilled person will appreciate that the binding parameters can be measured in the context of the monovalent or bivalent bi-, tri- or multispecific constructs. In certain embodiments, affinity values recited herein are determined in a Fab-scFv format, wherein the Fab targets CD3 and the scFv the pMHC.

In certain embodiments, the antigen binding protein is not a T cell receptor (TCR), including but not limited to, a soluble TCR. As used herein, the term “T cell receptor” or “TCR” refers to a heterodimeric protein comprised of two different chains (TCRα and TCRβ), which structurally belong to the immunoglobulin (Ig) superfamily. The extracellular portion of each chain is composed of variable (“Vα” and “Vβ”) and constant (“Cα” and “Cβ”) domains, and a hinge region, where the formation of a stabilizing disulfide bond occurs. The intracellular region forms a non-covalent interaction with another trans-membrane protein, CD3, which in the case of the correct target recognition leads to a series of conformational changes and a first T cell activation signal. Recognition and binding of peptide-MHC (pMHC) by a TCR is governed by the six hypervariable loops, termed complementarity determining regions (CDRs), located on the variable domains of the TCRα (CDRα1, CDRα2, CDRα3) and TCRβ (CDRβ1, CDRβ2, CDRβ3). CDR3 loops (CDRα3 and CDRβ3) lead the recognition of the processed antigen with the support of CDRα1 and CDRβ1, that have been implicated in the recognition of the N- and C-terminal amino acids of the presented peptide, respectively (Rudolph et al. Annu Rev Immunol. 24:419-66. 2006). Recognition of the MHC is typically achieved through the interaction with CDRα2 and CDRβ2. The high sequence diversity of the TCR is achieved through V(D)J recombination process, in which the variable domain is generated from a combination of genes: V (variable) and J (joining) for both TCRα and TCRβ, and an additional D (diversity) gene for TCRβ. The high antigen specificity of the TCR is controlled by the thymic maturation process, in which the self-reacting T cells are negatively selected. TCR affinity towards the specific pMHC and the functional avidity are the key factors controlling T-cell activation. A critical role in antigen recognition, however, is played by the affinity (K_{D}), i.e., the strength of binding between the TCR and the cell-displayed pMHC (Tian et al. J Immunol. 179:2952-2960. 2007). The physiological affinities of TCRs range from 1 μM to 100 μM (Davis et al. Annu Rev Immunol. 16:523-544. 1998), which, in comparison to antibodies, is relatively low.

As used herein, the term “peptide-MHC” or “pMHC” or “pMHC complex” as used interchangeably herein refers to a major histocompatibility complex (MHC) molecule (MHC-I or -II) with an antigenic peptide bound in a peptide binding pocket of the MHC. As is known in the art, MHC molecules present peptides, in particular antigenic peptides, on the surface of cells to be recognized by immune cells. Accordingly, as will be appreciated by a skilled artisan, the term “pMHC” as used herein refers to a complex of an MHC molecule and a peptide, in particular an antigenic peptide, presented by the MHC molecule. This is commonly known as MHC-restricted antigen presentation. Accordingly, the peptide targeted by the pMHC binding domains is an MHC-restricted peptide. The peptide can thus be considered as target peptide or target antigenic peptide. Further, in accordance with the present disclosure, the terms “target pMHC binding domain” and “pMHC binding domain” may be used interchangeably herein, and in any case refer to the at least first and at least second pMHC binding domains referred to herein throughout. The terms “target peptide/antigen presented by an MHC molecule/complex” and “MHC restricted target peptide/antigen”, or similar expressions used throughout the present specification, may be used interchangeably herein.

While MHCs occur in all vertebrates, the MHC in human is known as HLA (human leukocyte antigen). HLA is highly polygenic and can be broadly divided into three classes of MHC molecules, class I, class II and class III. Moreover, HLA genes have the highest level of polymorphism of the human genome. The target peptide may be presented on an MHC class I complex (such as of serotype HLA-A, HLA-B, HLA-C, HLA-E, HLA-F, HLA-G, HLA-K or HLA-L, or their respective subtypes) or an MHC class II complex (such as the serotypes HLA-DP, HLA-DQ, HLA-DR, DM or DO, or their respective subtypes). The HLA-A protein constitutes the alpha chain of the respective class I MHC (major histocompatibility complex) protein, which further comprises a beta 2 microglobulin subunit. The extracellular region of the alpha chain comprises three immunoglobulin-like domains (α1, α2, and α3). The α1 and α2 domains form a peptide-binding groove wherein the HLA-restricted peptide is typically located. Accordingly, peptides displayed by the HLA complex may be identified as pHLA herein. The terms “HLA-displayed”, “HLA-presented”, “loaded onto HLA”, “peptide-loaded HLA” and “HLA-restricted” are used interchangeably herein.

To reduce the complexity of the highly polymorphic restricting HLA genes, grouping different HLA molecules into clusters of several so-called HLA supertypes was suggested (see e.g., Wang M, Claesson M H. Classification of human leukocyte antigen (HLA) supertypes. Methods Mol Biol. 2014; 1184:309-17. Doi: 10.1007/978-1-4939-1115-8_17. PMID: 25048132; PMCID: PMC7121184). In each supertype, HLA molecules with similar peptide binding features are grouped into one supertype. It was hypothesized that if a peptide is able to bind to one allele within a supertype, it may also bind to all other alleles in this supertype. In practice, actually only a few peptides that are able to bind to one allele in a supertype can bind to all the other alleles within the supertype. In one aspect, the target peptides of the present invention are displayed by the A01 supertype, see for example Table 1 of Sidney et al., BMC Immunology, Vol. 9, Article number: 1 (2008). In certain embodiments, the target peptide is displayed by a serotype of the group consisting of HLA-A*01:01, HLA-A*26:01, HLA-A*26:02, HLA-A*26:03, HLA-A*30:02, HLA-A*30:03, HLA-A*30:04, and HLA-A*32:01.

In one aspect, the invention provides antigen binding proteins that bind to MHC-displayed NTDNNLAVY (SEQ ID NO: 54), in particular antibody fragments such as scFvs and larger bispecific formats including such scFvs, e.g. Fab-scFvs₂. Throughout the application, a pMHC is described by its HLA and target peptide, for example HLA-A*01:01/KK-LC-1 is the peptide of NTDNNLAVY (SEQ ID NO: 54) displayed on HLA-A*01:01. Similarly, HLA-A*01:01/PTS, or HLA-A*01:01/ICE1, for example, refer to the peptides of ETDNNIVVY (SEQ ID NO: 56), or NTDNLLTEY (SEQ ID NO: 55), respectively, being presented by HLA*01:01. In certain embodiments, the target pMHC complex wherein the peptide of NTDNNLAVY (SEQ ID NO: 54) is presented on an HLA-A*01:01 is herein also referred to as “HLA-A*01:01/KK-LC-1 antigen” or “HLA-A*01:01/KK-LC-1”.

In certain embodiments, provided are isolated antigen binding proteins that bind to MHC-displayed NTDNNLAVY (SEQ ID NO: 54), i.e., the isolated antigen binding protein is not associated or bound to the surface of a cell, such as a T cell. Isolated antigen binding proteins are separated from a component of its natural environment. In certain embodiments, the isolated antigen binding protein is purified to greater than 95% or 99% purity as determined by, for example, electrophoretic methods (e.g., SDS-PAGE, isoelectric focusing (IEF), capillary electrophoresis) or chromatography (e.g., ion exchange or reverse phase HPLC, affinity chromatography, size exclusion chromatography). In certain embodiments, the antigen binding protein is not a soluble TCR (e.g., a TCR lacking one or more of a transmembrane domain, an intracellular signaling domain, and constant domains). In certain embodiments, the antigen binding protein is a monoclonal antibody, in particular an antibody fragment such as a scFv.

As used herein, the term “PBS” refers to phosphate buffered saline. PBS is a pH-adjusted blend of phosphate buffers and saline solutions. In certain embodiments, the PBS comprises about 100-150 mM NaCl, about 1-5 mM KCl, about 1-10 mM Na₂HPO₄, and 1-5 mM KH₂PO₄. In certain embodiments, the PBS comprises 130 mM NaCl, 10 mM Na₂HPO₄, and pH of 6.0.

As used herein, the terms “first”, “second” or “third” when referring to antigen binding domains, are used solely for convenience to distinguish when there is more than one of each type of moiety. Use of these terms is not intended to imply any particular order, arrangement or orientation of the moieties unless explicitly so stated.

### Target Peptide-MHC and Antigen Binding Proteins Thereto

Described herein are antigen binding proteins, in particular isolated antibodies as well as artificial constructs comprising antibody fragments, that specifically recognize a target MHC-displayed NTDNNLAVY (SEQ ID NO: 54). The antigen binding proteins possess surprisingly high affinity while retaining high specificity for the target (i.e., low to no affinity for pMHCs displaying other targets, including off-target peptides, or beta-2-microglobulin (e.g., SEQ ID NO: 24). The antigen binding proteins of the disclosure show high specificity for KK-LC-1/HLA-A*01.

In one aspect, the antigen binding protein specifically binding MHC-displayed NTDNNLAVY (SEQ ID NO: 54) comprises: (a) an antibody heavy chain variable (VH) domain comprising an HCDR1 amino acid sequence of SSYYYMC (SEQ ID NO: 25), an HCDR2 amino acid sequence of CIYAGSSGSTYYASWAKG (SEQ ID NO: 26), and an HCDR3 amino acid sequence of GAGYGNGGHSL (SEQ ID NO: 27); and (b) an antibody light chain variable (VL) domain comprising an LCDR1 amino acid sequence of RASKNIYNSLA (SEQ ID NO: 28), an LCDR2 amino acid sequence of GASNLES (SEQ ID NO: 29), and an LCDR3 amino acid sequence of QATYYGHDNIGGA (SEQ ID NO: 30).

In certain embodiments, the MHC displaying a peptide of the disclosure, e.g., the target peptide or an off-target peptide, is of the HLA-A*01 supertype. In certain embodiments, the peptide is displayed by a serotype of the group consisting of HLA-A*01:01, HLA-A*26:01, HLA-A*26:02, HLA-A*26:03, HLA-A*30:02, HLA-A*30:03, HLA-A*30:04, and HLA-A*32:01, in particular HLA-A*01:01.

In some embodiments, the antigen binding protein is not a T Cell Receptor (TCR). In some embodiments, the antigen binding protein is an antibody or a fragment thereof.

The binding profile of the antigen binding protein was determined which allows for the identification of potential off-target peptides, e.g., in mass-spectrometry databases containing peptides presented in healthy tissues, as well as for the identification of the positions that are most critical for binding recognition. In one embodiment, target binding is impacted by amino acid residues 4, 5, 6, 7 and 8 of the MHC-displayed NTDNNLAVY peptide (SEQ ID NO: 54). In some embodiments, target binding is further impacted by amino acid position 9 of NTDNNLAVY (SEQ ID NO: 54).

In some embodiments, the EC50-value of Granzyme B (GrzB) expression is increased by at least 10-fold when at least one of amino acid position 5, 6, 7 and 8 of the MHC-displayed NTDNNLAVY peptide (SEQ ID NO: 54) is substituted by alanine, serine, arginine and/or aspartic acid. In some embodiments, said EC50-value is increased by at least 20-fold, at least 50-fold, at least 100-fold, or by at least 1000-fold. In some embodiments, the substitutions is selected from the group consisting of N5A, L6A, A7S, V8A, N5R, L6R, A7R, V8R, NSD, L6D, A7D or V8D of NTDNNLAVY (SEQ ID NO: 54).

Granzyme B (GrzB) expression can be determined by methods known in the art, see e.g., the MSD used in Example 11. Assay kits are commercially available and can be performed according to the manufacturer's instructions. From the data generated therewith, the skilled person can calculate the EC50-value.

Knowledge of the binding profile allows for the identification of potential off-target peptides. Off-target peptides expressed in healthy tissues may pose a cross-reactivity risk. In some embodiments, an off-target peptide has no more than 2, 3, 4, or 5 amino acid mismatches from NTDNNLAVY (SEQ ID NO: 54). In some embodiments, the pMHC binding proteins of the disclosure show a significant reduction in affinity to an HLA-restricted off-target peptide, compared to HLA-restricted NTDNNLAVY (SEQ ID NO: 54). In some embodiments, the antigen binding protein shows low or no binding to MHC-displayed off-target peptides. Low affinity binding of an antibody refers to weak, transient interactions between the antibody and its target antigen, characterized by a higher dissociation constant K_{D} compared to high-affinity antibodies. Accordingly, the antibody binds less tightly and releases the antigen more readily. As used herein, “no binding affinity” refers to no detectable binding affinity relative to background with one or more binding affinity assays known in the art, such as, but not limited to, an ELISA assay. “low binding” or weak binding as used herein refers to a K_{D} value of about 1 mM (10⁻³ M) to 400 nM (10⁻⁹ M), as determined by SPR. In specific embodiments, a “low binding” affinity (K_{D}) value is about 400 nM or higher (i.e., the higher the K_{D} value, the weaker the binding), such as e.g. at least 500 nM, 600 nM, 700, nM, 800 nM, 900 nM, or at least about 1 μM, as determined by SPR.

In certain embodiments, the antigen binding protein selectively binds a pMHC complex of a given HLA subtype and a target peptide, but not to a pMHC complex of the same HLA subtype presenting a different peptide, in particular off-target peptide.

In certain embodiments, the antigen binding protein specifically binds the NTDNNLAVY (SEQ ID NO: 54) displayed by HLA-A*01 on the surface of a cell, such as a cancer cell. In certain embodiments, the cell is a T2 cell expressing HLA-A*01 that has been pulsed with the target peptide NTDNNLAVY (SEQ ID NO: 54). In yet other embodiments, the cell is a cancer cell that endogenously presents the target peptide NTDNNLAVY (SEQ ID NO: 54) by an HLA complex (i.e., without pulsing).

In some embodiments, an HLA-restricted off-target peptide is derived from the gene product of ICE1, DSG3, PTS, FUS, PTPRC, ROBO1, TBL3, CHTOP, DMXL2, FAP, GPAM, PDE10A, VAV1, ZBTB40, ZNF430, SRP72, PTPN7, ESYT1, WDR81 and/or SLC25A10. In certain embodiments, said peptides are displayed by an HLA of supertype A*01, in particular by HLA-A*01:01.

In certain embodiments, the HLA-restricted ICE1 derived peptide consists of NTDNLLTEY (SEQ ID NO: 55). In certain embodiments, the HLA-restricted DSG3 derived peptide consists of YTDNWLAVY (SEQ ID NO: 57). In certain embodiments, the HLA-restricted PTS derived peptide consists of ETDNNIVVY (SEQ ID NO: 56). In certain embodiments, the HLA-restricted FUS derived peptide consists of NSDNNTIFV (SEQ ID NO: 59). In certain embodiments, the HLA-restricted PTPRC derived peptide consists of NLDKNLIKY (SEQ ID NO: 60). In certain embodiments, the HLA-restricted ROBO1 derived peptide consists of NSDSNLTTY (SEQ ID NO: 61). In certain embodiments, the HLA-restricted TBL3 derived peptide consists of TADHNLLLY (SEQ ID NO: 62). In certain embodiments, the HLA-restricted CHTOP derived peptide consists of QLDNQLDAY (SEQ ID NO: 63). In certain embodiments, the HLA-restricted DMXL2 derived peptide consists of SVDSNLFVY (SEQ ID NO: 64). In certain embodiments, the HLA-restricted FAP derived peptide consists of SADNNIVLY (SEQ ID NO: 65). In certain embodiments, the HLA-restricted GPAM derived peptide consists of RTERNVAVY (SEQ ID NO: 66). In certain embodiments, the HLA-restricted PDE10A derived peptide consists of GGDNQLLLY (SEQ ID NO: 67). In certain embodiments, the HLA-restricted VAV1 derived peptide consists of NLDQSLAHY (SEQ ID NO: 68). In certain embodiments, the HLA-restricted ZBTB40 derived peptide consists of FTDNQILLK (SEQ ID NO: 69). In certain embodiments, the HLA-restricted ZNF430 derived peptide consists of GADRNLLVY (SEQ ID NO: 70). In certain embodiments, the HLA-restricted SRP72 derived peptide consists of RTEEALQLY (SEQ ID NO: 71). In certain embodiments, the HLA-restricted PTPN7 derived peptide consists of FLHHTLALY (SEQ ID NO: 72). In certain embodiments, the HLA-restricted ESYT1 derived peptide consists of VTQESKAVY (SEQ ID NO: 73). In certain embodiments, the HLA-restricted WDR81 derived peptide consists of MSEEHTAVY (SEQ ID NO: 74). In certain embodiments, the HLA-restricted SLC25A10 derived peptide consists of RTDGILALY (SEQ ID NO: 75).

As shown in the experimental section, a TBL3 derived peptide (TADHNLLLY; SEQ ID NO: 62) has been identified as having similarity to the KK-LC-1 target peptide NTDNNLAVY (SEQ ID NO: 54) and being highly expressed among many healthy tissues and immune cells. In one aspect, the antigen binding protein of the disclosure specifically binds to the major histocompatibility complex (MHC)-displayed NTDNNLAVY (SEQ ID NO: 54) and has at least about a 10-fold reduction in affinity to MHC-displayed TADHNLLLY (SEQ ID NO: 62), relative to affinity to MHC-displayed NTDNNLAVY (SEQ ID NO: 54). In some embodiments, the antigen binding protein has at least a 50-fold decrease in affinity (e.g., a 75-fold, 80-fold, 90-fold, 100-fold, 120-fold, 150-fold, or 200-fold decrease in affinity) for the MHC-displayed peptide TADHNLLLY (SEQ ID NO: 62), relative to MHC-displayed NTDNNLAVY (SEQ ID NO: 54), as determined by SPR.

In some embodiments, the antigen binding protein has at least a 50-fold decrease in affinity (e.g., a 75-fold, 80-fold, 90-fold, 100-fold, 120-fold, 150-fold, or 200-fold decrease in affinity) for the MHC-displayed peptides selected from the group consisting of YTDNWLAVY (SEQ ID NO: 57), ETDNNIVVY (SEQ ID NO: 56), NTDNLLTEY (SEQ ID NO: 55), NSDSNLTTY (SEQ ID NO: 61), NSDNNTIFV (SEQ ID NO: 59), NLDKNLIKY (SEQ ID NO: 60), QLDNQLDAY (SEQ ID NO: 63), SVDSNLFVY (SEQ ID NO: 64), SADNNIVLY (SEQ ID NO: 65), GGDNQLLLY (SEQ ID NO: 67), NLDQSLAHY (SEQ ID NO: 68), FTDNQILLK (SEQ ID NO: 69), GADRNLLVY (SEQ ID NO: 70), RTEEALQLY (SEQ ID NO: 71), FLHHTLALY (SEQ ID NO: 72), VTQESKAVY (SEQ ID NO: 73), MSEEHTAVY (SEQ ID NO: 74) and RTDGILALY (SEQ ID NO: 75), relative to affinity to MHC-displayed NTDNNLAVY (SEQ ID NO: 54), as determined by SPR.

In certain embodiments, said antigen binding protein has an affinity (K_{D}) to MHC-displayed NTDNNLAVY (SEQ ID NO: 54) of at least about 30 nM or lower (i.e., stronger binding, as reflected by a lower numerical K_{D} value), such as at least about 25 nM, 20 nM, 19 nM, about 18 nM, about 17 nM, about 16 nM, about 15 nM, about 14 M, about 13 nM, about 12 nM, about 11 nM, about 10 nM, about 9 nM, about 8 nM, about 7 nM, about 6 nM about 5 nM, about 4 nM, about 3 nM, about 2 nM, e.g., about 1.8 nM.

In certain embodiments, said antigen binding protein binds to MHC-displayed RTERNVAVY (SEQ ID NO: 66), a GPAM derived off-target peptide, with an affinity (K_{D}) of at least about 200 nM or higher (i.e. weaker binding), such as at least about 250 nM, 300 nM, 350 nM, 400 nM, 450 nM, 470 nM, 500 nM, 550 nM, 590 nM, such as about 594 nM. In specific embodiments, the antigen binding protein binds to MHC-displayed RTERNVAVY (SEQ ID NO: 66) with an affinity (KD) of about 450 nM to 600 nM.

In some embodiments, the antigen binding protein specifically targeting MHC-displayed NTDNNLAVY (SEQ ID NO: 54) comprises a variable light chain sequence comprising or consisting of SEQ ID NO: 32 or a variant thereof having at least 90%, 91%, 92%, 93%, 94%, 95%, 96%, 97%, 98% or 99% identity thereto.

In some embodiments, the antigen binding protein specifically targeting MHC-displayed NTDNNLAVY (SEQ ID NO: 54) comprises a variable heavy chain sequence comprising or consisting of SEQ ID NO: 31 or a variant thereof having at least 96%, 97%, 98% or 99% identity thereto.

Hence, on one aspect, an antigen binding protein specifically targeting MHC-displayed NTDNNLAVY (SEQ ID NO: 54) is provided, comprising: a) a variable heavy chain sequence comprising or consisting of an amino acid sequence that is at least 90%, 95%, 96%, 97%, 98%, 99% or 100% identical to SEQ ID NO: 31; and/or (b) a variable light chain sequence comprising or consisting of an amino acid sequence that is at least 90%, 95%, 96%, 97%, 98%, 99% or 100% identical to SEQ ID NO: 32.

In certain embodiments, the antigen binding protein is a variant of said SEQ ID NO: 31 and SEQ ID NO: 32. In certain embodiments, the variant comprises substitutions, such as 1, 2, 3, 4, 5, 6, 7, 8, 9 or more substitutions. In certain embodiments, such substitutions are within the CDRs. In certain embodiments, such substitutions are within the framework regions. In certain embodiments, the variant sequence of the variable light chain comprising or consisting of SEQ ID NO: 32 is at least 90%, 95%, 96%, 97%, 98%, or 99% identical to SEQ ID NO: 32. In certain embodiments, said variant variable light chain comprises an LCDR1 amino acid sequence of RASKNIYNSLA (SEQ ID NO: 28), an LCDR2 amino acid sequence of GASNLES (SEQ ID NO: 29), and an LCDR3 amino acid sequence of QATYYGHDNIGGA (SEQ ID NO: 30). In certain embodiments, the variant sequence of the variable heavy chain comprising or consisting of SEQ ID NO: 31 is at least 90%, 95%, 96%, 97%, 98%, or 99% identical to SEQ ID NO: 31. In certain embodiments, said variant variable heavy chain comprises an HCDR1 amino acid sequence of SSYYYMC (SEQ ID NO: 25), an HCDR2 amino acid sequence of CIYAGSSGSTYYASWAKG (SEQ ID NO: 26), and an HCDR3 amino acid sequence of GAGYGNGGHSL (SEQ ID NO: 27).

In some embodiments, the antigen binding protein is or comprises an antibody, such as a full-length immunoglobulin or an antibody fragment. In some embodiments, the antibody fragment is a Fab, a Fab′, a F(ab′)₂, a scFv, a Fv fragment or a scFab.

In some embodiments, the VH and VL are joined with an amino acid linker. In some embodiments, the amino acid linker comprises the amino acid sequence of SEQ ID NO: 2, SEQ ID NO: 3, SEQ ID NO: 4, SEQ ID NO: 5, SEQ ID NO: 6, or SEQ ID NO: 7, in particular SEQ ID NO: 6. In certain embodiments, the VH and VL are joined to form a scFv. In an scFv according to the present disclosure, the VL and VH arrangement can be either VL-linker-VH or VH-linker-VL, with the former orientation being the preferred one. In certain embodiments, the antigen binding protein is or comprises a scFv having the amino acid sequence of SEQ ID NO: 33, or a variant thereof having at least 80%, 85%, 90%, 91%, 92%, 93%, 94%, 95%, 96%, 97%, 98% or 99% sequence identity thereto. In certain embodiments, the antigen binding protein is a variant of said scFv, comprising substitutions, such as 1, 2, 3, 4, 5, 6, 7, 8, 9, 10, 11, 12, 13, 14, 15, 16, 17, 18, 19, 20 or more substitutions. In certain embodiments, the one or more substitutions are made within the VH domain, the VL domain and/or the linker. In certain embodiments, a substitution is within a CDR sequence. In certain embodiments, such substitutions are within the framework regions of the VH or VL domain.

In certain embodiments, said scFv has a melting temperature of about 75° C. A high melting temperature indicates greater resistance to thermal denaturation, which is desirable for stability in physiological conditions.

In certain embodiments, said scFv has a human germline identity of at least 85%. For determination, see e.g. Example 13. Such high level of human germline identity is desirable for therapeutic applications to reduce immunogenicity.

TABLE 2
The sequences disclosed herein are:
Linkers

| | |
|---|---|
| SEQ ID NO: 1 | (GGGGS)n wherein n = 1, 2, 3, 4 or 5 |
| SEQ ID NO: 2 | GGGGS |
| SEQ ID NO: 3 | GGGGSGGGGS |
| SEQ ID NO: 4 | GGGGSGGGGSGGGGS |
| SEQ ID NO: 5 | GGGGSGGGGSGGGGSGGGGS |
| SEQ ID NO: 6 | GGGGGSGGGGSGGGGSGGGGS |
| SEQ ID NO: 7 | GGGGSGGGGSGGGGSGGGGAS |
| SEQ ID NO: 8 | DGGGS |
| SEQ ID NO: 9 | TGEKP |
| SEQ ID NO: 10 | GGRR |
| SEQ ID NO: 11 | EGKSSGSGSESKVD |
| SEQ ID NO: 12 | KESGSVSSEQLAQFRSLD |
| SEQ ID NO: 13 | GGRRGGGS |
| SEQ ID NO: 14 | LRQRDGERP |
| SEQ ID NO: 15 | LRQKDGGGSERP |
| SEQ ID NO: 16 | GSTSGSGKPGSGEGSTKG |
| SEQ ID NO: 17 | EPKSC |
| SEQ ID NO: 18 | EPKSCGGGGS |
| SEQ ID NO: 19 | EPKSCDKTHT |
| SEQ ID NO: 20 | EPKSCDKTHTGGGGS |
| SEQ ID NO: 21 | DKTHT |
| SEQ ID NO: 22 | DKTHTGGGGS |

MHC related sequences

| | | |
|---|---|---|
| SEQ ID NO: 23 | HLA-A*01:01 | GSHSMRYFFTSVSRPGRGEPRFIAVGYVDD |
| | extracellular | TQFVRFDSDAASQKMEPRAPWIEQEGPEY |
| | domain | WDQETRNMKAHSQTDRANLGTLRGYYNQ |
| | | SEDGSHTIQIMYGCDVGPDGRFLRGYRQDA |
| | | YDGKDYIALNEDLRSWTAADMAAQITKRK |
| | | WEAVHAAEQRRVYLEGRCVDGLRRYLEN |
| | | GKETLQRTDPPKTHMTHHPISDHEATLRCW |
| | | ALGFYPAEITLTWQRDGEDQTQDTELVETR |
| | | PAGDGTFQKWAAVVVPSGEEQRYTCHVQH |
| | | EGLPKPLTLRWE |
| SEQ ID NO: 24 | human β2m | IQRTPKIQVYSRHPAENGKSNFLNCYVSGFH |
| | | PSDIEVDLLKNGERIEKVEHSDLSFSKDWSF |
| | | YLLYYTEFTPTEKDEYACRVNHVTLSQPKI |
| | | VKWDRDM |

pMHC antigen binding protein related sequences

| | | |
|---|---|---|
| SEQ ID NO: 25 | HCDR1 | SSYYYMC |
| SEQ ID NO: 26 | HCDR2 | CIYAGSSGSTYYASWAKG |
| SEQ ID NO: 27 | HCDR3 | GAGYGNGGHSL |
| SEQ ID NO: 28 | LCDR1 | RASKNIYNSLA |
| SEQ ID NO: 29 | LCDR2 | GASNLES |
| SEQ ID NO: 30 | LCDR3 | QATYYGHDNIGGA |
| SEQ ID NO: 31 | VH M3904 | EVQLVESGGGSVQPGGSLRLSCAASGIDFSS |
| | | YYYMCWVRQAPGKCLEWIGCIYAGSSGST |
| | | YYASWAKGRFTISRDNSKNTLYLQMNSLR |
| | | AEDTATYYCARGAGYGNGGHSLWGQGTT |
| | | VTVSS |
| SEQ ID NO: 32 | VL M3904 | DIQMTQSPSTLSASVGDRVTITCRASKNIYN |
| | | SLAWYQQKPGKAPKLLIYGASNLESGVPSR |
| | | FSGSGSGTDYTLTISSLQPEDSATYYCQATY |
| | | YGHDNIGGAFGCGTKVEIK |
| SEQ ID NO: 33 | scFv M3904 | DIQMTQSPSTLSASVGDRVTITCRASKNIYN |
| | | SLAWYQQKPGKAPKLLIYGASNLESGVPSR |
| | | FSGSGSGTDYTLTISSLQPEDSATYYCQATY |
| | | YGHDNIGGAFGCGTKVEIKGGGGSGGGGS |
| | | GGGGSGGGGSEVQLVESGGGSVQPGGSLR |
| | | LSCAASGIDFSSYYYMCWVRQAPGKCLEWI |
| | | GCIYAGSSGSTYYASWAKGRFTISRDNSKN |
| | | TLYLQMNSLRAEDTATYYCARGAGYGNG |
| | | GHSLWGQGTTVTVSS |

CD3 binding domain

| | | |
|---|---|---|
| SEQ ID NO: 34 | HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSC |
| SEQ ID NO: 35 | LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECS |
| SEQ ID NO: 36 | VH | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSS |
| SEQ ID NO: 37 | VL | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLG |
| SEQ ID NO: 38 | HCDR1 | STYAMN |
| SEQ ID NO: 39 | HCDR2 | RIRSKYNNYATYYADSVKG |
| SEQ ID NO: 40 | HCDR3 | HGNFGDSYVSWFAY |
| SEQ ID NO: 41 | LCDR1 | GSSTGAVTTSNYAN |
| SEQ ID NO: 42 | LCDR2 | GTNKRAP |
| SEQ ID NO: 43 | LCDR3 | ALWYSNHWV |

Bispecific antigen binding proteins

| | | |
|---|---|---|
| SEQ ID NO: 44 | M2883 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSEIVM |
| | | TQSPSTLSASVGDRVIITCQASENIYNSLAW |
| | | YQQKPGKAPKLLIYGASNLESGVPSRFSGS |
| | | GSGTEYTLTISSLQPDDFATYYCQSTYYGH |
| | | DNVGGAFGCGTKLTVLGGGGGSGGGGSGG |
| | | GGSGGGGSEVQLVESGGGSVQPGGSLRLSC |
| | | TVSGIDFSSYYYMCWVRQAPGKCLEWIGCI |
| | | YAGSSGSTYYASWAKGRFTISKDTSKNTVY |
| | | LQMNSLRAEDTATYYCARGAGYGNDGHSL |
| | | WGQGTTVTVSS |
| SEQ ID NO: 45 | M2883 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECS |
| SEQ ID NO: 46 | M2769 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSEIVM |
| | | TQSPSTLSASVGDRVIITCQASENIYNSLAW |
| | | YQQKPGKAPKLLIYGASNLESGVPSRFSGS |
| | | GSGTEFTLTISSLQPDDFATYYCQSTYYGHD |
| | | NVGGAFGCGTKLTVLGGGGGSGGGGSGGG |
| | | GSGGGGSEVQLVESGGGSVQPGGSLRLSCT |
| | | VSGIDFSSYYYMCWVRQAPGKCLEWIGCIY |
| | | AGSSGSTYYASWAKGRFTISKDTSKNTVYL |
| | | QMNSLRAEDTATYYCARGAGYGNDGHSL |
| | | WGQGTTVTVSS |
| SEQ ID NO: 47 | M2769 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECS |
| SEQ ID NO: 48 | M2759 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSQQQL |
| | | EESGGDLVKPGASLTLTCKASGIDFSSYYY |
| | | MCWVRQAPGKGLEWIACIYAGSSGSTYYA |
| | | SWAKGRFTISKTSSTMVTLQMTSLTAADTA |
| | | TYFCARGAGYGNDGHSLWGPGTLVTVSSG |
| | | GGGSGGGGSGGGGSGGGGSSDPMLTQTPA |
| | | SVSAAVGGTVTIKCQASENIYNSLAWYQQK |
| | | PGQRPKLLIYGASNLESGVPSRFKGSGSGTE |
| | | YTLTISDLEADDAATYYCQSTYYGHDNVG |
| | | GAFGGGTEVVVR |
| SEQ ID NO: 49 | M2759 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECS |
| SEQ ID NO: 50 | M3591 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSTLSASVGDRVTITCRASKNIYNSLAW |
| | | YQQKPGKAPKLLIYGASNLESGVPSRFSGS |
| | | GSGTDYTLTISSLQPEDFATYYCQSTYYGH |
| | | DNVGGAFGCGTKVEIKGGGGSGGGGSGGG |
| | | GSGGGGSEVQLVESGGGSVQPGGSLRLSCA |
| | | ASGIDFSSYYYMCWVRQAPGKCLEWIGCIY |
| | | AGSSGSTYYASWAKGRFTISRDNSKNTLYL |
| | | QMNSLRAEDTATYYCARGAGYGNDGHSL |
| | | WGQGTTVTVSS |
| SEQ ID NO: 51 | M3591 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECS |
| SEQ ID NO: 52 | M3904 HC | EVQLVESGGGLVQPGGSLRLSCAASGFTFS |
| | | TYAMNWVRQAPGKGLEWVGRIRSKYNNY |
| | | ATYYADSVKGRFTISRDDSKNTLYLQMNSL |
| | | RAEDTAVYYCVRHGNFGDSYVSWFAYWG |
| | | QGTLVTVSSASTKGPSVFPLAPSSKSTSGGT |
| | | AALGCLVKDYFPEPVTVSWNSGALTSGVH |
| | | TFPAVLQSSGLYSLSSVVTVPSSSLGTQTYIC |
| | | NVNHKPSNTKVDKKVEPKSCGGGGSDIQM |
| | | TQSPSTLSASVGDRVTITCRASKNIYNSLAW |
| | | YQQKPGKAPKLLIYGASNLESGVPSRFSGS |
| | | GSGTDYTLTISSLQPEDSATYYCQATYYGH |
| | | DNIGGAFGCGTKVEIKGGGGSGGGGSGGG |
| | | GSGGGGSEVQLVESGGGSVQPGGSLRLSCA |
| | | ASGIDFSSYYYMCWVRQAPGKCLEWIGCIY |
| | | AGSSGSTYYASWAKGRFTISRDNSKNTLYL |
| | | QMNSLRAEDTATYYCARGAGYGNGGHSL |
| | | WGQGTTVTVSS |
| SEQ ID NO: 53 | M3904 LC | QAVVTQEPSLTVSPGGTVTLTCGSSTGAVT |
| | | TSNYANWVQQKPGKSPRGLIGGTNKRAPG |
| | | VPARFSGSLLGGKAALTISGAQPEDEADYY |
| | | CALWYSNHWVFGGGTKLTVLGQPKAAPSV |
| | | TLFPPSSEELQANKATLVCLISDFYPGAVTV |
| | | AWKADSSPVKAGVETTTPSKQSNNKYAAS |
| | | SYLSLTPEQWKSHRSYSCQVTHEGSTVEKT |
| | | VAPTECSGGGGSDIQMTQSPSTLSASVGDR |
| | | VTITCRASKNIYNSLAWYQQKPGKAPKLLI |
| | | YGASNLESGVPSRFSGSGSGTDYTLTISSLQ |
| | | PEDSATYYCQATYYGHDNIGGAFGCGTKV |
| | | EIKGGGGSGGGGSGGGGSGGGGSEVQLVE |
| | | SGGGSVQPGGSLRLSCAASGIDFSSYYYMC |
| | | WVRQAPGKCLEWIGCIYAGSSGSTYYASW |
| | | AKGRFTISRDNSKNTLYLQMNSLRAEDTAT |
| | | YYCARGAGYGNGGHSLWGQGTTVTVSS |

Target peptide

| | | |
|---|---|---|
| SEQ ID NO: 54 | Target KK-LC- | NTDNNLAVY |
| | 1 peptide | |
| SEQ ID NO: 126 | off-target | ASDRNIVLY |
| | peptide | |
| | DCAF13 | |

### Dual Peptide-MHC—Immune Cell Engaging Antigen Binding Proteins

In one aspect, the disclosure provides an antigen binding protein, comprising (i) a first antigen binding domain comprising the antigen binding protein targeting MHC-displayed NTDNNLAVY (SEQ ID NO: 54) described supra (i.e., a “pMHC binding domain” or “KK-LC-1 binding domain” as used interchangeably herein), and (ii) at last one antigen binding domain with specificity for a cell surface protein of an immune cell (e.g., CD3 on the surface of a T cell; an “immune cell binding domain”). Such antigen binding protein is multispecific, e.g., bispecific or trispecific. In some embodiments, said antigen binding protein is at least bispecific.

In some embodiments, the multispecific antigen binding protein further comprises a second antigen binding domain, such as a second pMHC binding domain, in particular a KK-LC-1 binding domain targeting MHC-displayed NTDNNLAVY (SEQ ID NO: 54).

Targeting two pMHC complexes on the surface of a target cell (e.g., a cancer cell) improves target cell engagement through avidity-enhanced binding. The enhanced binding (i.e., lower apparent K_{D} of the multivalent interaction) may in turn promote improved target cell killing relative to an antigen binding protein that only has one pMHC binding domain. The avidity-enhanced binding created by at least two pMHC binding domains may be particularly useful when targeting pMHC complexes of high copy number on the surface of a target cell (e.g., cancer cell).

As used herein, the terms “first”, “second” or “third” with respect to antigen binding domains are used for convenience of distinguishing when there is more than one antigen binding domain. Unless explicitly stated otherwise, the use of these terms is not intended to denominate a specific order or orientation. In some embodiments, the first target pMHC complex and the second target pMHC complex are the same (i.e., they have identical sequences and the multispecific antigen binding protein is in fact bispecific). In certain embodiments, the first target pMHC complex and the second target pMHC complex are different (e.g., each complex comprises a different peptide bound to the MHC molecule or the same peptide is bound to a different HLA subtype). In some embodiments, the first pMHC binding domain and the second pMHC binding domain are different (i.e., the binding domains bind to different epitopes). In certain embodiments, the first pMHC binding domain and the second pMHC binding domain are the same (i.e., the binding domains bind to the same epitope and/or are identical in sequence). In certain embodiments thereof, the antigen binding protein is bispecific and bivalent for the pMHC, i.e., the antigen binding protein targets both an immune cell target and MHC-displayed NTDNNLAVY (SEQ ID NO: 54) and at the same time bivalent for MHC-displayed NTDNNLAVY (SEQ ID NO: 54).

In some embodiments, any one or more of the first and/or second pMHC binding domain and the immune cell binding domain is or comprises a full-length immunoglobulin or an antibody fragment. In some embodiments, the antibody fragment is or comprises a Fab fragment, a F(ab′)₂ fragment, a Fab′ fragment, an Fv fragment, a single chain variable fragment (scFv), or a single domain antibody fragment. In some embodiments, the first antigen binding domain and/or the second antigen binding domain is or comprises a scFv, in particular the scFv of SEQ ID NO: 33 or a variant thereof. In certain embodiments, the antigen binding protein does not comprise an Fc domain. In certain embodiments, such antigen binding protein lacking an Fc domain is a Fab-sdAb, a Fab-(sdAb)₂, a Fab-scFv, a scFv-Fab-scFv (Fab-scFv₂), a F(ab′)₂ fragment, a bis-scFv (or tandem scFv or BiTE), a DART, diabodies, a scDb, a tribody, a (scFv)₃, a tetrabody, Fab₂, Fab₃, Fab₄ or MATCH, in particular a scFv-Fab-scFv (Fab-scFv₂).

In certain embodiments, the immune cell is a human cell. In certain embodiments, the immune cell is selected from the group consisting of a T cell, a B cell, a natural killer (NK) cell, a natural killer T (NKT) cell, a neutrophil cell, a monocyte, and a macrophage. In certain embodiments, the immune cell is a T cell. In certain embodiments, the cell surface protein of an immune cell is selected from the group consisting of CD3, TCRα, TCRβ, the α/β T Cell Receptor, CD16 (e.g., CD16a), NKG2D, CD94/NKG2C, NKp30, NKp46, CD89, CD64, and CD32 (e.g., CD32a) on the surface of an immune cell. In certain embodiments, the cell surface protein of an immune cell is CD3.

In certain embodiments, the immune cell binding domain is an antibody, such as a CD3 targeting antibody, a CD16a targeting antibody or BMA031 or a variant thereof. BMA031 has been described to have specificity for a common determinant on the TCR αβ/CD3 complex (see e.g., Borst J et al, Hum Immunol 1990 November; 29 (3): 175-88) and is commercially available from different sources.

The term “CD3” refers to any native CD3 from any vertebrate source, including primates. In certain embodiments, the immune cell binding domain specifically binds to human CD3, in particular to the CD38 (epsilon) chain/subunit of CD3 (see e.g., UniProt (www.uniprot.org) accession no. P07766 (version 189), or NCBI (www.ncbi.nlm.nih.gov/) RefSeq NP_000724.1.) The CD3 molecule may be the full-length, unprocessed CD3 molecule or a fragment or variant thereof that e.g., results from processing in the cell. For example, such variants may be naturally occurring variants like splice variants or allelic variants. In certain embodiments, the antigen binding proteins disclosed herein bind to an epitope of CD3 that is conserved among the CD3 antigens from different species, such as non-human primates (e.g., cynomolgus monkeys) or rodents (e.g., mice, rats). In certain embodiments, the antigen binding proteins are not cross-reactive with a CD3 antigen from rodents (e.g., mouse or rat) or minipigs.

Suitable anti-CD3 binding domains are known in the art, particularly T-cell activating CD3-epsilon binding domains. The terms “CD3 binding domain” and “anti-CD3 binding domain” are used interchangeably herein. In certain embodiments of the present disclosure, the anti-CD3 binding domain is any one of antibodies SP34, OKT3 or UCHT1, or a variant sequence thereof having at least about 80%, 85%, 90%, 95%, 96%, 97%, 98% or 99% identity thereto, while retaining the same specificity as its parent. SP34, OKT3 or UCHT1 are murine antibodies; for therapeutic applications, humanized versions of SP34, OKT3 or UCHT1, i.e., huSP34, huOKT3 or huUCHT1, are preferred. In certain embodiments, the humanized variant sequence of SP34, OKT3 or UCHT1 is optimized for use in Fab format. For example, the humanized huSP34, huOKT3 or huUCHT1 may comprise 1, 2, 3, 4, 5, 6, 7, 8, 9 or more substitutions while retaining selective binding to CD3. Exemplary CD3 binding domains are disclosed in U.S. Pat. No. 6,750,325, WO2008079713, U.S. Pat. No. 7,635,475, WO2005040220, U.S. Pat. No. 7,728,114, WO9404679, U.S. Pat. No. 7,381,803, WO2008119567, WO2014110601, WO2014145806, WO2015095392, WO2016086189 and/or WO2019195535A1, each of which is incorporated herein by reference. Fab, VH, VL and CDR sequences of exemplary CD3 binding domains are shown in Table 2. Multispecific antigen binding proteins of the disclosure targeting at least the pMHC complex and CD3 are also termed “T cell engaging proteins”.

Thus, in one embodiment, the antigen binding protein of the disclosure is a T cell engaging protein, comprising at least a first domain targeting MHC-displayed NTDNNLAVY (SEQ ID NO: 54) and second domain targeting CD3, wherein the first domain comprises an antibody heavy chain variable (VH) domain comprising the HCDR amino acid sequences of SEQ ID NOs: 25, 26, and 27; and an antibody light chain variable (VL) domain comprising the LCDR amino acid sequences of SEQ ID NOs.: 28, 29, and 30; and the second domain targeting CD3, in particular a huSP34, huOKT3 or huUCHT1derived antigen binding protein.

In some embodiments, the CD3 binding domain comprises: (a) an antibody heavy chain variable (VH) domain comprising an HCDR1 amino acid sequence of STYAMN (SEQ ID NO: 38) an HCDR2 amino acid sequence of RIRSKYNNYATYYADSVKG (SEQ ID NO: 39), and an HCDR3 amino acid sequence of HGNFGDSYVSWFAY (SEQ ID NO: 40); and (b) an antibody light chain variable (VL) domain comprising an LCDR1 amino acid sequence of GSSTGAVTTSNYAN (SEQ ID NO: 41), an LCDR2 amino acid sequence of GTNKRAP (SEQ ID NO: 42), and an LCDR3 amino acid sequence of ALWYSNHWV (SEQ ID NO: 43).

Thus, in certain embodiments, a T cell engaging protein is provided, comprising (1) a CD3 binding domain specifically binding to CD3, and (2) a KK-LC-1 binding domain specifically binding to a major histocompatibility complex (MHC)-displayed peptide NTDNNLAVY (SEQ ID NO: 54), wherein:
(1) the CD3 binding domain comprises: (a) an antibody heavy chain variable (VH) domain comprising an HCDR1 amino acid sequence of STYAMN (SEQ ID NO: 38), an HCDR2 amino acid sequence of RIRSKYNNYATYYADSVKG (SEQ ID NO: 39), and an HCDR3 amino acid sequence of HGNFGDSYVSWFAY (SEQ ID NO: 40); and (b) an antibody light chain variable (VL) domain comprising an LCDR1 amino acid sequence of GSSTGAVTTSNYAN (SEQ ID NO: 41), an LCDR2 amino acid sequence of GTNKRAP (SEQ ID NO: 42), and an LCDR3 amino acid sequence of ALWYSNHWV (SEQ ID NO: 43); and
(2) the KK-LC-1 binding domain comprises: (a) an antibody heavy chain variable (VH) domain comprising an HCDR1 amino acid sequence of SSYYYMC (SEQ ID NO: 25), an HCDR2 amino acid sequence of CIYAGSSGSTYYASWAKG (SEQ ID NO: 26), and an HCDR3 amino acid sequence of GAGYGNGGHSL (SEQ ID NO: 27); and (b) an antibody light chain variable (VL) domain comprising an LCDR1 amino acid sequence of RASKNIYNSLA (SEQ ID NO: 28), an LCDR2 amino acid sequence of GASNLES (SEQ ID NO: 29), and an LCDR3 amino acid sequence of QATYYGHDNIGGA (SEQ ID NO: 30).

In certain embodiments, a CD3 binding domain of the disclosure comprises: (a) an antibody heavy chain variable (VH) domain comprising or consisting of the amino acid sequence of SEQ ID NO: 36 or a variant thereof that is at least about 90%, 91%, 92%, 93%, 94%, 95%, 96%, 97%, 98% or 99% identical thereto; and/or (b) an antibody light chain variable (VL) domain comprising the amino acid sequence of SEQ ID NO: 37 or a variant thereof that is at least about 90%, 91%, 92%, 93%, 94%, 95%, 96%, 97%, 98% or 99% identical thereto. In particular embodiments, such variant comprises an HCDR sequences of SEQ ID NOs: 38, 39 and 40, and the LCDRs sequences of SEQ ID NOs: 41, 42, and 43.

Thus, in certain embodiments, an antigen binding protein of the disclosure (i.e., a T cell engaging protein) comprises a CD3 binding domain and at least one KK-LC-1 binding domain specifically binding to a major histocompatibility complex (MHC)-displayed peptide NTDNNLAVY (SEQ ID NO: 54), wherein:
(1) the CD3 binding domain comprises: (a) an antibody heavy chain variable (VH) domain comprising or consisting of the amino acid sequence of SEQ ID NO: 36 or a variant thereof that is at least about 90%, 91%, 92%, 93%, 94%, 95%, 96%, 97%, 98% or 99% identical thereto; and (b) an antibody light chain variable (VL) domain comprising the amino acid sequence of SEQ ID NO: 37 or a variant thereof that is at least about 90%, 91%, 92%, 93%, 94%, 95%, 96%, 97%, 98% or 99% identical thereto; and
(2) the KK-LC-1 binding domain comprises: (a) an antibody heavy chain variable (VH) domain comprising an HCDR1 amino acid sequence of SSYYYMC (SEQ ID NO: 25), an HCDR2 amino acid sequence of CIYAGSSGSTYYASWAKG (SEQ ID NO: 26), and an HCDR3 amino acid sequence of GAGYGNGGHSL (SEQ ID NO: 27); and (b) an antibody light chain variable (VL) domain comprising an LCDR1 amino acid sequence of RASKNIYNSLA (SEQ ID NO: 28), an LCDR2 amino acid sequence of GASNLES (SEQ ID NO: 29), and an LCDR3 amino acid sequence of QATYYGHDNIGGA (SEQ ID NO: 30).

Thus, in certain embodiments, an antigen binding protein of the disclosure (i.e., a T cell engaging protein) comprises a CD3 binding domain and at least one KK-LC-1 binding domain specifically binding to a major histocompatibility complex (MHC)-displayed peptide NTDNNLAVY (SEQ ID NO: 54), wherein:
(1) the CD3 binding domain comprises: (a) an antibody heavy chain variable (VH) domain comprising or consisting of the amino acid sequence of SEQ ID NO: 36 or a variant thereof that is at least about 90%, 91%, 92%, 93%, 94%, 95%, 96%, 97%, 98% or 99% identical thereto; and (b) an antibody light chain variable (VL) domain comprising the amino acid sequence of SEQ ID NO: 37 or a variant thereof that is at least about 90%, 91%, 92%, 93%, 94%, 95%, 96%, 97%, 98% or 99% identical thereto; and
(2) the KK-LC-1 binding domain comprises: (a) an antibody heavy chain variable (VH) domain comprising the amino acid sequence of SEQ ID NO: 31 or a variant thereof that is at least about 90%, 95%, 96%, 97%, 98% or 99% identical thereto; and (b) an antibody light chain variable (VL) domain comprising the amino acid sequence of SEQ ID NO: 32 or a variant thereof that is at least about 90%, 95%, 96%, 97%, 98% or 99% identical thereto.

Exemplary formats of bispecific antigen binding proteins are or comprise a (scFv)₂, BiTE, BIKE, Dart, diabody, Fab₂, or a Fab-scFv. In certain embodiments thereof, said multispecific antigen binding protein is bispecific and monovalent for each target, e.g., monovalent for CD3 and KK-LC-1. In certain embodiments, said first antigen binding domain (i.e. the KK-LC-1 binding domain comprising the antigen binding protein detailed supra), may e.g., be any one of a scFv, scFab, a diabody, a Fv fragment or a Fab format. In certain embodiments, the first antigen binding domain comprises an scFv and the immune cell binding domain targeting the cell surface protein of an immune cell comprises a Fab.

In some embodiments, the CD3 binding domain is or comprises a Fab fragment, wherein said Fab fragment comprises: (1) a heavy chain (HC) comprising a CH1 domain and said VH; and (2) a light chain (LC) comprising a CL domain and said VL.

For example, in certain embodiments, said Fab fragment comprises or consists of (1) the HC of SEQ ID NO: 34, or variant thereof having at least 90%, 91%, 92%, 93%, 94%, 95%, 96%, 97%, 98% or 99% sequence identity thereto; and (2) the LC of SEQ ID NO: 35, or variant thereof having at least 90%, 91%, 92%, 93%, 94%, 95%, 96%, 97%, 98% or 99% sequence identity thereto.

Hence, in one aspect, the disclosure provides a multispecific antigen binding protein comprising:
a) a Fab domain which specifically binds to a cell surface protein of an immune cell, the Fab domain comprising a heavy chain and a light chain (e.g., an immune cell binding domain);
b) at least a first pMHC binding domain operably linked to the heavy chain, wherein the first pMHC binding domain binds to first target peptide-MHC (pMHC) complex; and
c) at least a second pMHC binding domain operably linked to the light chain, wherein the second pMHC binding domain binds to a second pMHC complex, wherein the first and/or the second pMHC binding domain is the MHC-displayed NTDNNLAVY (SEQ ID NO: 54) binding protein as described above.

In some embodiments, the antigen binding protein has no more than two pMHC binding domains, i.e., is limited with regard to pMHC binding domains to one first pMHC binding domain one second pMHC binding domain, in particular when both pMHC binding domains are in scFv format and are identical in sequence.

In certain embodiments, the first pMHC binding domain is operably linked to the C-terminus of the Fab heavy chain or the N-terminus of the Fab heavy chain. In certain embodiments, the first pMHC binding domain is operably linked to the C-terminus of the Fab light chain or the N-terminus of the Fab light chain.

In certain embodiments, the second pMHC binding domain is operably linked to the C-terminus of the Fab heavy chain or the N-terminus of the Fab heavy chain. In certain embodiments, the second pMHC binding domain is operably linked to the C-terminus of the Fab light chain or the N-terminus of the Fab light chain.

The at least first and/or the at least second pMHC binding domain may both be linked to either the heavy chain, or may both be linked to the light chain of the Fab domain. In some embodiments, the at least first and the at least second pMHC binding domain are not linked to the same chain of the Fab domain, i.e., one is linked to the heavy chain of the Fab domain, and the other is linked to the light chain of the Fab domain. For example, in certain embodiments, the at least first pMHC binding domain is operably linked to the C-terminus of the heavy chain of the Fab domain, and the at least second pMHC binding domain is operably linked to the C-terminus of the light chain of the Fab domain. In other embodiments, the at least first pMHC binding domain is operably linked to the C-terminus of the heavy chain of the Fab domain, and the at least second pMHC binding domain is operably linked to the N-terminus of the light chain of the Fab domain.

The Fab domain of the multispecific antigen binding protein of the disclosure may serve as a specific heterodimerization scaffold to which the additional pMHC binding domains are linked. The natural and efficient heterodimerization properties of the heavy chain (Fd fragment) and light chain (LC) of a Fab fragment makes the Fab fragment a useful scaffold. Additional binding domains may be in several different formats, including, but not limited to, another Fab domain, a scFv, or an sdAb.

Each chain of the Fab fragment can be extended at the N- or C-terminus with additional binding domains. The chains may be co-expressed in mammalian cells, where the host-cell Binding immunoglobulin protein (BiP) chaperone drives the formation of the heavy chain-light chain heterodimer (Fd:LC). These heterodimers are stable, with each of the binders retaining their specific affinities. The one or two remaining pMHC binding domains may then be fused e.g., as scFvs or sdAbs to distinct Fab chains where each chain can be extended, e.g., at the C-terminus with an additional scFv or sdAb domain (see, for example, Schoonjans et al. J. Immunology, 165 (12): 7050-7057, 2000; Schoonjans et al. Biomolecular Engineering, 17:193-202, 2001.) An additional advantage of using Fabs as a heterodimerization unit is that Fab molecules are abundantly present in serum and therefore may be non-immunogenic when administered to a subject.

In some embodiments, a) the first antigen binding domain is or comprises an scFv being linked to the C-terminus of the Fab domain heavy chain and the second antigen binding domain comprises an scFv being linked to the C-terminus of the Fab domain light chain;
b) the first antigen binding domain is or comprises an scFv being linked to the N-terminus of the Fab domain heavy chain and the second antigen binding domain comprises an scFv being linked to the N-terminus of the Fab domain light chain;
c) the first antigen binding domain is or comprises an scFv being linked to the N-terminus of the Fab domain heavy chain and the second antigen binding domain comprises an scFv being linked to the C-terminus of the Fab domain light chain; or
d) the first antigen binding domain is or comprises an scFv being linked to the C-terminus of the Fab domain heavy chain and the second antigen binding domain comprises an scFv being linked to the N-terminus of the Fab domain light chain.

In certain embodiments, the Fab domain comprises five amino acids of an antibody hinge region, in particular the sequence stretch up to the first cysteine of the antibody hinge region, The presence of cysteine allows for an additional disulfide bridge which may further stabilize the antigen binding protein. In certain embodiments, said 5 amino acids are located at the C-terminus of the heavy chain of the Fab CH1, In certain embodiments, said sequence stretch is or comprises the sequence EPKSC (SEQ ID NO: 17). In certain embodiments, said at least 5 amino acids of an antibody hinge region comprises EPKSCDKTHT (SEQ ID NO: 19). The antibody hinge region may additionally comprise the sequence GGGGS (SEQ ID NO: 2) which may serve as a linker sequence to the pMHC binding domain(s). Thus, in some embodiments, a pMHC binding domain is linked to the C-terminal end of the Fab CH1 domain via any of EPKSCGGGGS (SEQ ID NO: 18), EPKSCDKTHT (SEQ ID NO: 19), EPKSCDKTHTGGGGS (SEQ ID NO: 20), DKTHT (SEQ ID NO: 21), DKTHTGGGGS (SEQ ID NO: 22) or GGGGSGGGGS (SEQ ID NO: 3) linker, in particular of EPKSCGGGGS (SEQ ID NO: 18).

In certain embodiments, the Fab CL domain is followed by a linker, such as GGGGS (SEQ ID NO: 2).

In certain embodiments, a first scFv is bound to the CH1 domain of the Fab via EPKSCGGGGS (SEQ ID NO: 18) and a second scFv is bound to the CL domain via GGGGS (SEQ ID NO: 2).

In certain embodiments, the immune cell binding domain, in particular the Fab domain, specifically binds to CD3 with an affinity (K_{D}) between about 1 nM to about 150 nM (e.g., about 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 61 nM, 62 nM, 63 nM, 64 nM, 65 nM, 66 nM, 67 nM, 68 nM, 69 nM, 70 nM, 71 nM, 72 nM, 73 nM, 74 nM, 75 nM, 76 nM, 77 nM, 78 nM, 79 nM, 80 nM, 81 nM, 82 nM, 83 nM, 84 nM, 85 nM, 86 nM, 87 nM, 88 nM, 89 nM, 90 nM, 91 nM, 92 nM, 93 nM, 94 nM, 95 nM, 96 nM, 97 nM, 98 nM, 99 nM, 100 nM, 101 nM, 102 nM, 103 nM, 104 nM, 105 nM, 106 nM, 107 nM, 108 nM, 109 nM, 110 nM, 111 nM, 112 nM, 113 nM, 114 nM, 115 nM, 116 nM, 117 nM, 118 nM, 119 nM, 120 nM, 121 nM, 122 nM, 123 nM, 124 nM, 125 nM, 126 nM, 127 nM, 128 nM, 129 nM, 130 nM, 131 nM, 132 nM, 133 nM, 134 nM, 135 nM, 136 nM, 137 nM, 138 nM, 139 nM, 140 nM, 141 nM, 142 nM, 143 nM, 144 nM, 145 nM, 146 nM, 147 nM, 148 nM, 149 nM, 150 nM), as determined by SPR. In certain embodiments, the immune cell binding domain, in particular the Fab domain, specifically binds to CD3 with an affinity (K_{D}) between about 1 nM to about 50 nM, as determined by SPR. In certain embodiments, the immune cell binding domain, in particular the Fab domain, specifically binds to CD3 with an affinity (K_{D}) between about 20 nM to about 50 nM, as determined by SPR. In certain embodiments, the immune cell binding domain, in particular the Fab domain, specifically binds to CD3 with an affinity (KD) between about 1 nM to about 10 nM, e.g., about 3 nM, as determined by SPR.

In certain embodiments, the immune cell binding domain, in particular the Fab domain, specifically binds to CD3 with an affinity (K_{D}) of about 1 nM, of about 10 nM, or of about 50 nM, as determined by SPR.

In some embodiments, the association rate constant kₐ of the anti-CD3 binding domain is between about 1×10⁵ to about 1×10⁷ M⁻¹s⁻¹, such as at least 1×10⁶ M⁻¹s⁻¹ or at least 2×10⁶ M⁻¹s⁻¹.

In some embodiments, the dissociation rate constant k_{d} of the anti-CD3 binding domain is between about 1×10⁻¹ to about 1×10⁻⁶ s⁻¹, such as at least 2×10⁻³ s⁻¹, or at least 3×10⁻³ s⁻¹ or at least 4×10⁻³ s⁻¹. Without being bound to theory, a fast dissociation rate, e.g., a k_{d}-value of 2-3×10⁻³ s⁻¹, may lead to less T cell overactivation and in consequence, less cytokine release.

In one embodiment, the association rate constant kₐ and/or the dissociation rate constant k_{d} are equivalent or similar for both CD3-heterodimers CD3εγ (epsilon/gamma) and CD388 (epsilon/delta), i.e., there is no significant difference for either the kₐ or the k_{d} or both of the anti-CD3 binding domain to CD3εγ (epsilon/gamma) and CD3εδ (epsilon/delta) when measured under the same conditions, in particular when determined by SPR at 25° C. In certain embodiments, the association rate constant kₐ and/or the dissociation rate constant k_{d} values that are within 1-fold of each other, 1.5-fold of each other, 2-fold of each other, 2.5-fold of each other or 3-fold of each other, i.e., association rate constant kₐ values of 1×10⁵ M⁻¹s⁻¹ and 3×10⁵ M⁻¹s⁻¹.

Bispecific antigen binding proteins as provided herein, such as the ones described above targeting both MHC-displayed NTDNNLAVY (SEQ ID NO: 54) and CD3 on the surface of an immune cell, may be in a Fab-scFv format. In certain embodiments, said pMHC binding domain is operably linked to the C-terminus of the Fab heavy chain or the N-terminus of the Fab heavy chain. In certain embodiments, said pMHC binding domain is operably linked to the C-terminus of the Fab light chain or the N-terminus of the Fab light chain.

In certain embodiments, a bispecific antigen binding protein of the disclosure (i.e., CD3× KK-LC-1 binding protein) is a Fab-scFv comprising: (1) a HC comprising or consisting of an amino acid sequence that is at least about 90%, 91%, 92%, 93%, 94%, 95%, 96%, 97%, 98%, 99% or 100% identical to the amino acid sequence of SEQ ID NO: 52; and a LC comprising of an amino acid sequence that is at least about 90%, 91%, 92%, 93%, 94%, 95%, 96%, 97%, 98%, 99% or 100% identical to the amino acid sequence of SEQ ID NO: 35. In certain embodiments, said Fab-scFv comprises (a) an antibody heavy chain variable (VH) domain comprising an HCDR1 amino acid sequence of SSYYYMC (SEQ ID NO: 25), an HCDR2 amino acid sequence of CIYAGSSGSTYYASWAKG (SEQ ID NO: 26), and an HCDR3 amino acid sequence of GAGYGNGGHSL (SEQ ID NO: 27); and (b) an antibody light chain variable (VL) domain comprising an LCDR1 amino acid sequence of RASKNIYNSLA (SEQ ID NO: 28), an LCDR2 amino acid sequence of GASNLES (SEQ ID NO: 29), and an LCDR3 amino acid sequence of QATYYGHDNIGGA (SEQ ID NO: 30). In certain embodiments, the Fab-scFv further comprises (a) an antibody heavy chain variable (VH) domain comprising an HCDR1 amino acid sequence of STYAMN (SEQ ID NO: 38), an HCDR2 amino acid sequence of RIRSKYNNYATYYADSVKG (SEQ ID NO: 39), and an HCDR3 amino acid sequence of HGNFGDSYVSWFAY (SEQ ID NO: 40); and (b) an antibody light chain variable (VL) domain comprising an LCDR1 amino acid sequence of GSSTGAVTTSNYAN (SEQ ID NO: 41), an LCDR2 amino acid sequence of GTNKRAP (SEQ ID NO: 42), and an LCDR3 amino acid sequence of ALWYSNHWV (SEQ ID NO: 43).

In certain embodiments, a bispecific antigen binding protein of the disclosure (i.e., CD3× KK-LC-1 binding protein) is a Fab-scFv comprising the scFv of SEQ ID NO: 33, being linked to the C-terminus of the HC of SEQ ID NO: 34 and a LC of SEQ ID NO: 35.

In one embodiment, the antigen binding protein of the disclosure comprises a Fab domain as well as a first and a second pMHC binding domain, both pMHC binding domains being scFvs, i.e., the molecule is a Fab-(scFv)₂ (or “Fab-scFv₂” as interchangeably used herein). An advantage of the Fab-scFv₂ scaffolds of the disclosure is the intermediate molecular size of approximately 75-110 kDa. Blinatumomab, a bispecific T cell engager (BiTE), has shown excellent results in patients with relapsed or refractory acute lymphoblastic leukemia. Because of its small size (60 kDa), blinatumomab is characterized by a short serum half-life of several hours, and therefore continuous infusion is needed (see, U.S. Pat. No. 7,112,324 B1). The antigen binding proteins of the disclosure are expected to have significantly longer half-lives in comparison to smaller bispecific antibodies, such as BiTEs like blinatumomab, and thus, do not require continuous infusion due to their favorable half-life. An intermediate sized molecule may avoid kidney clearance and provide a half-life sufficient for improved tumor accumulation. While the antigen binding proteins of the disclosure have increased plasma half-life compared to other small bispecific formats, they still retain the tumor penetration ability. On the other hand, the molecules of the instant disclosure lacking an Fc domain are expected to have a shorter half-life than larger molecules including an Fc domain. A prolonged half-life may overstimulate T cells and lead to T cell exhaustion. Also, especially in solid tumors, a large molecular weight may translate into a lower degree of tumor penetration. In some embodiments, the in vivo half-life is of about 7 days.

In certain embodiments, the antigen binding protein comprises a molecular weight of about 75 kDa to about 110 kDa (e.g., about 75 kDa, about 80 kDa, about 85 kDa, about 90 kDa, about 95 kDa, about 100 kDa, about 105 kDa or about 110 kDa). In certain embodiments, the antigen binding protein has increased serum half-life relative to an antigen binding protein with a molecular weight of less than about 60 kDa. In certain embodiments, the bispecific antigen binding protein in Fab-(scFv)2 format has a molecular weight of 100 kDa, and is predicted to achieve a favorable pharmacokinetic (PK) likely allowing dosing every two to three weeks.

In certain embodiments, the antigen binding protein is a bispecific Fab-(scFv)₂ and comprises (i) a single Fab domain which specifically binds to CD3 with an affinity (K_{D}) from about 1 nM to about 50 nM, (ii) a first pMHC binding scFv linked to the C-terminus of the Fab domain heavy chain and (iii) a second pMHC binding scFv linked to the C-terminus of the Fab domain light chain, wherein both pMHC binding scFvs have an affinity (K_{D}) of at least about 30 nM to about 1.8 nM to the target pMHC complex.

In some embodiments, the multispecific antigen binding protein is a bispecific Fab-(scFv)₂ comprising: a HC comprising or consisting of an amino acid sequence that is at least about 80%, 85%, 90%, 91%, 92%, 93%, 94%, 95%, 96%, 97%, 98%, 99% or 100% identical to the amino acid sequence of SEQ ID NO: 52; and a LC comprising or consisting of an amino acid sequence that is at least about 80%, 85%, 90%, 91%, 92%, 93%, 94%, 95%, 96%, 97%, 98%, 99% or 100% identical to the amino acid sequence of SEQ ID NO: 53. In certain embodiments, said HC comprises an HCDR1 amino acid sequence of SSYYYMC (SEQ ID NO: 25), an HCDR2 amino acid sequence of CIYAGSSGSTYYASWAKG (SEQ ID NO: 26), and an HCDR3 amino acid sequence of GAGYGNGGHSL (SEQ ID NO: 27), and/or said LC comprises an LCDR1 amino acid sequence of RASKNIYNSLA (SEQ ID NO: 28), an LCDR2 amino acid sequence of GASNLES (SEQ ID NO: 29), and an LCDR3 amino acid sequence of QATYYGHDNIGGA (SEQ ID NO: 30). In certain embodiments, the HC further comprises an HCDR1 amino acid sequence of STYAMN (SEQ ID NO: 38), an HCDR2 amino acid sequence of RIRSKYNNYATYYADSVKG (SEQ ID NO: 39), and an HCDR3 amino acid sequence of HGNFGDSYVSWFAY (SEQ ID NO: 40); and/or the LC further comprises an LCDR1 amino acid sequence of GSSTGAVTTSNYAN (SEQ ID NO: 41), an LCDR2 amino acid sequence of GTNKRAP (SEQ ID NO: 42), and an LCDR3 amino acid sequence of ALWYSNHWV (SEQ ID NO: 43).

Accordingly, the present disclosure encompasses, in certain embodiments, a bispecific bivalent antigen binding protein, comprising a Fab domain which specifically binds to CD3; and no more than two pMHC binding domains, wherein both pMHC binding domains are targeting the same HLA-A*01 complex (i.e., the antigen binding protein is bivalent with regard to the target pMHC complex), such as the same MHC-displayed NTDNNLAVY (SEQ ID NO: 54), or are targeting the same peptide presented by a MHC, in particular presented by a MHC-displayed NTDNNLAVY (SEQ ID NO: 54) of HLA-A*01:01 complex, wherein both pMHC binding domains are each a scFv, and wherein one of the two pMHC binding domains is operably linked to the C-terminus of the heavy chain of the CD3 binding domain, and the other pMHC binding domain is operably linked to the C-terminus or the N-terminus of the light chain of the CD3 binding domain.

Said bispecific Fab-scFv₂ is thus a humanized, affinity-matured, and stability-engineered T cell engager that showed an overall safe profile in the presence of normal human primary cells, see e.g., Example 14. In certain embodiments, binding of the bispecific antigen binding protein to CD3 on a T cell, in particular on a cytotoxic T lymphocyte, triggers a cellular response of T cell. The so activated T cell may for example exhibit altered proliferation, differentiation, cytokine secretion, cytotoxic effector molecule release, cytotoxic activity, and expression of activation markers. Assays for measuring T cell activation are known to the skilled person. None of the off-target peptides identified caused relevant T cell activation in presence of the bispecific bivalent antigen binding protein when presented on TAP-deficient, (empty) HLA-A*01-expressing T2 cells. Furthermore, bispecific bivalent antigen binding protein did not induce relevant T cell reactivity towards a panel of HLA-A*01 positive normal human cells from different organs and tissues. These results support a low risk for off-target reactivity and a favorable safety profile of the bispecific bivalent antigen binding proteins of the disclosure.

In certain embodiments, and as exemplified in the Examples and the drawings, the antigen binding proteins of the disclosure, in particular the T cell engaging proteins, show an overall safe profile in the presence of normal human primary cells.

The antigen binding proteins of the disclosure, in particular the T cell engaging proteins, induce efficient cytotoxicity of KK-LC-1 positive/HLA-A*01 positive tumor cells, e.g., NSCLC cell lines NCI-H1703 and/or EKVX, in co-cultures with human PBMCs, see e.g., Examples 10 and 14.

In certain embodiments, the antigen binding proteins of the disclosure, in particular the T cell engaging proteins, show favorable drug-like biophysical properties, (as e.g. characterized by solubility, shelf-life stability and/or stability at physiological conditions) and have a favorable manufacturability profile (as e.g., characterized by titers ≥3.0 g/L achieved in CHO pools and/or protein quality as evidenced by SEC, CEX and/or HIC).

In certain embodiments, antigen binding proteins of the disclosure, in particular the T cell engaging proteins, are stable under accelerated conditions at 50 mg/ml and 37° C. for one week, not showing aggregation or oligomerization above 1% as evidenced by SEC. In certain embodiments, the antigen binding protein has a melting temperature higher than about 60° C., such as at least about 65° C., at least about 70° C. or at least about 75° C., such as e.g. about 78° C. A high melting temperature, i.e., over 60° C., indicates greater resistance to thermal denaturation, which is desirable for stability in physiological conditions.

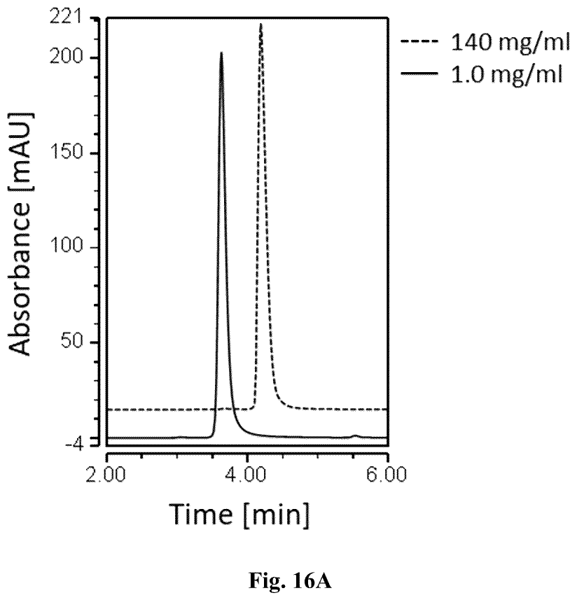

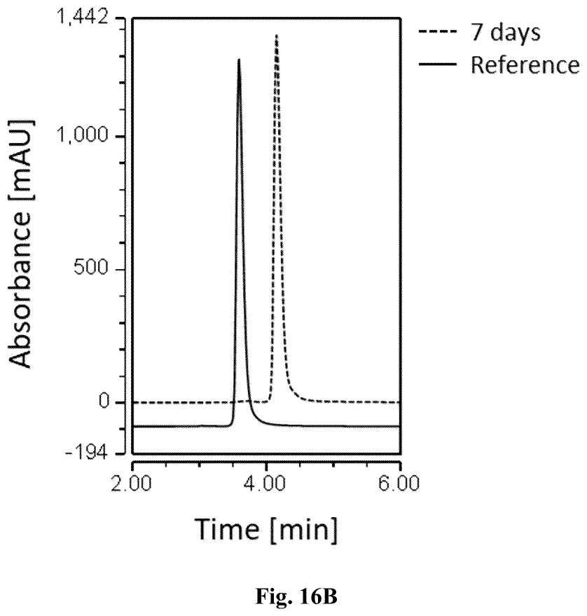

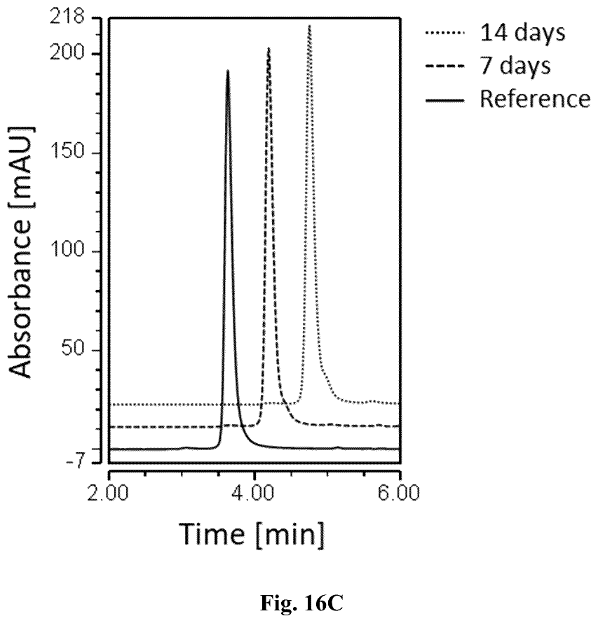

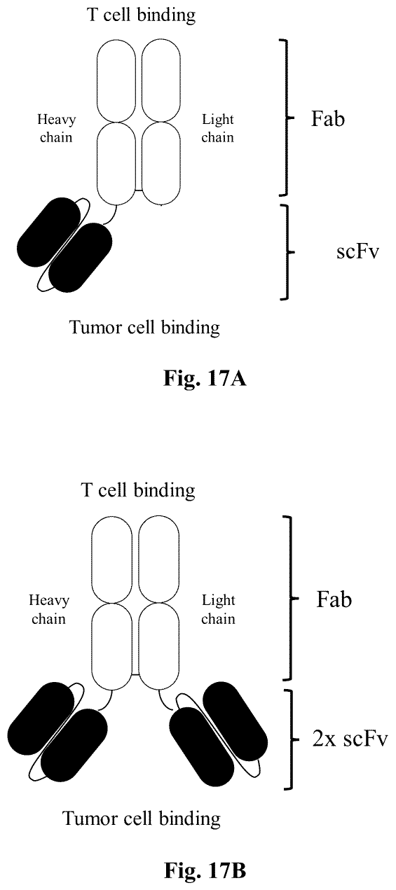

In certain embodiments, antigen binding proteins of the disclosure, in particular the T cell engaging proteins, are stable in artificial subcutaneous fluid buffer (see Schuster et al. 2021) at simulated physiological conditions (34° C., pH 7.4) for at least 2 weeks, with little to no degradation as evidenced by SEC., (see e.g., Example 14 and FIG. 16.

In certain embodiments, the antigen binding proteins of the disclosure, in particular the T cell engaging proteins, retains stability and remains at least 94%, 95%, 96%, 97%, 98%, 99% or 100% monomeric after incubation for 14 days at 4° C. in PBS at 1 mg/ml and/or 10 mg/ml as determined by SEC-HPLC.

In certain embodiments, the antigen binding proteins of the disclosure, in particular the T cell engaging proteins, can be concentrated to at least 100 mg/ml, at least 120 mg/ml or at least 140 mg/ml without any detectable oligomerization as measured by SEC (see e.g., Example 14 and FIG. 16).

In certain embodiments, the antigen binding proteins of the disclosure, in particular the T cell engaging proteins, reach titers of ≥3.0 g/L in CHO pools.

In certain embodiments, upon cancer cell killing mediated by the antigen binding proteins of the disclosure, in particular the T cell engaging proteins, are capable of activating T cells, as e.g. evidenced by increased expression of activation markers (e.g., CD69, CD25) and proliferation markers (e.g., Ki67) CD3+ cells and/or the release of cytolytic molecules in culture supernatants, including IFNγ, IL-2, TNFα, IL-6, IL-10 and/or granzyme B. confirming that the antigen binding proteins of the disclosure efficiently kill KK-LC-1 positive/HLA-A*01 positive tumor cells in vitro. See e.g., Example 14. In certain embodiments, the antigen binding proteins of the disclosure, in particular of the T cell engaging proteins, induce the release of cytokines and cytolytic molecules in vitro when co-cultured with the NSCLC cell line NCI-H1703, such as IFNγ, IL-2, IL-6, Granzyme B, IL-10 and/or TNF alpha. The bispecific antigen binding protein are thus capable of efficiently killing KK-LC-1 positive/HLA-A*01 positive tumor cells in vitro.

The antigen binding proteins of the disclosure, in particular the T cell engaging proteins are capable of activating and inducing proliferation of CD8+ T cells. see e.g., Example 14.

In certain embodiments, the antigen binding proteins of the disclosure, in particular the T cell engaging proteins, has cytotoxic activity against an MHC-displaying NTDNNLAVY (SEQ ID NO: 54) cell. In certain embodiments, the multispecific antigen binding comprises at least one additional binding domain targeting an immune cell. In certain embodiments, the T cell engaging protein lacks detectable cytotoxic activity against a non-NTDNNLAVY (SEQ ID NO: 54) pMHC presenting cell.

### Variants and Competitor Proteins

As contemplated throughout the description, also encompassed are variants of the sequences disclosed herein. A variant amino acid or nucleic acid sequence differs from its parental sequence e.g., by virtue of insertion (including addition), deletion and/or substitution of one or more amino acid residues or nucleobases, respectively, while retaining at least one desired property of the parent sequence disclosed herein, e.g., specific antigen binding, efficacy on target positive tumor cells, stability (e.g., serum stability, thermal stability, and/or storage stability), producibility (e.g., expression levels), safety (e.g., reactivity in healthy tissues, Granzyme B release, proinflammatory cytokine IFN gamma antigen-positive or antigen-negative cell lines, and/or low to no induction of pro-inflammatory cytokines IL-2, IL-6 and TNF alpha cytokine release), efficacy (e.g., tumor growth inhibition, tumor eradication, and/or T cell activating properties as e.g. determined in vitro), or binds MHC-displayed NTDNNLAVY (SEQ ID NO: 54) to a similar affinity range). In certain embodiments, the variant antigen binding protein retains specific binding to the target pMHC complex of at least 50%, such as 60%, 70%, 80%, 90% or 95% of the equilibrium dissociation constant K_{D} of the reference antigen binding protein (i.e., the corresponding antigen binding protein without said substitutions, insertions, and/or deletions) when measured under identical conditions. In certain embodiments, the variant shows stronger binding to the target peptide than the reference antigen binding protein. Variants may be artificially engineered or naturally occurring, such as e.g., allelic or splice variants. In some embodiments, the variant antigen binding protein comprises an amino acid sequence being at least about 80%, 85%, 90%, 95%, 96%, 97%, 98% or 99% identical to an amino acid sequence disclosed herein. In certain embodiments, the variant antigen binding protein comprises 1, 2, 3, 4, 5, 6, 7, 8, 9, or 10 or more substitutions. In certain embodiments, the variant antigen binding protein comprises a substitution in the VH of the KK-LC-1 binding protein selected from the group consisting of A23T, A24V, S30R, R71K, N73T, L78V and G100aD (Kabat nomenclature); and/or a substitution in the VL of the KK-LC-1 binding protein selected from the group consisting of DIE, Q3V, T20I, R24Q, K27E, K42Q, A43R, S63K, D70E, Y71F, S77D, Q79E, P80A, E81D, S83F, S83A, A90S, 195cV, C100G, K103E, V104L, E105V, E105T, 1106V, K107R and K107L (Kabat nomenclature).

In certain aspects, the disclosure provides an antigen binding protein which specifically binds to a major histocompatibility complex (MHC)-displayed NTDNNLAVY (SEQ ID NO: 54), comprising: (a) an antibody heavy chain variable (VH) domain comprising or consisting of the amino acid sequence SEQ ID NO: 31, or a variant thereof that is at least about 90%, 95%, 96%, 97%, 98%, or 99% identical to SEQ ID NO: 31; and (b) an antibody light chain variable (VL) domain comprising or consisting of the amino acid sequence of SEQ ID NO: 32, or a variant thereof that is at least about 90%, 95%, 96%, 97%, 98%, or 99% identical to SEQ ID NO: 32, wherein such variant antigen binding protein:
(i) binds to MHC-displayed NTDNNLAVY (SEQ ID NO: 54) with an affinity (K_{D}) of at least about 30 nM or stronger; and/or
(ii) has at least a 50-fold decrease in affinity for the MHC-displayed peptides of SEQ ID NOs: 55-57 and 59-75, relative to the affinity to MHC-displayed NTDNNLAVY (SEQ ID NO: 54).

In certain aspects, the disclosure provides a bispecific Fab-scFv₂ which specifically binds to a major histocompatibility complex (MHC)-displayed NTDNNLAVY (SEQ ID NO: 54) and CD3, comprising: (a) a HC comprising or consisting of the amino acid sequence of SEQ ID NO: 52, or a variant thereof that is at least about 80%, 85%, 90%, 95%, 96%, 97%, 98%, or 99% identical to SEQ ID NO: 52; and (b) a LC comprising or consisting of the amino acid sequence of SEQ ID NO: 53, or a variant thereof that is at least about 80%, 85%, 90%, 95%, 96%, 97%, 98%, or 99% identical to SEQ ID NO: 53, wherein such variant bispecific Fab-scFv₂:
(i) binds to MHC-displayed NTDNNLAVY (SEQ ID NO: 54) with an affinity (K_{D}) of at least about 30 nM or stronger; and/or
(ii) has at least a 50-fold decrease in affinity for the MHC-displayed peptides of SEQ ID NOs: 55-57 and 59-75, relative to the affinity to MHC-displayed NTDNNLAVY (SEQ ID NO: 54).

In certain embodiments, a variant as described herein (i) has at least 10-fold decrease in affinity (e.g., a 50-fold, 75-fold, 80-fold, 90-fold, 100-fold, 120-fold, 150-fold, or 200-fold decrease in affinity) for the MHC-displayed peptides selected from the group consisting of SEQ ID NO: 55, SEQ ID NO: 56, SEQ ID NO: 57, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74 and SEQ ID NO: 75, relative to MHC-displayed NTDNNLAVY (SEQ ID NO: 54), as determined by SPR; (ii) has at least a 50-fold decrease in affinity (e.g., a 75-fold, 80-fold, 90-fold, 100-fold, 120-fold, 150-fold, or 200-fold decrease in affinity) for the MHC-displayed peptide TADHNLLLY (SEQ ID NO: 62), relative to MHC-displayed NTDNNLAVY (SEQ ID NO: 54), as determined by SPR; and/or (iii) has a binding profile dictated by at least 4 amino acid residues in said MHC-displayed NTDNNLAVY (SEQ ID NO: 54), such that the EC50-value of Granzyme B (GrzB) release is increased by at least a 10-fold when the MHC-displayed NTDNNLAVY (SEQ ID NO: 54) comprises a substitution selected from the group consisting of N5A, L6A, A7S, V8A, N5R, L6R, A7R, V8R, N5D, L6D, A7D or V8D.

In certain embodiments, a variant antigen binding protein retains specific binding to the target pMHC or CD3, respectively, and/or competes with the antigen binding protein disclosed herein for binding to its target.

In certain embodiments, the antigen binding proteins disclosed herein have at least one desired property over a competitor protein against the same target (i.e., MHC-displayed NTDNNLAVY (SEQ ID NO: 54)), e.g., specific antigen binding, efficacy on target positive tumor cells, stability (e.g., serum stability, thermal stability, and/or storage stability), producibility (e.g., expression levels), safety (e.g., reactivity in healthy tissues, Granzyme B release or proinflammatory cytokine IFN gamma on antigen-negative cell lines, and/or low to no induction of pro-inflammatory cytokines IL-2, IL-6 and TNF alpha cytokine release), efficacy (e.g., tumor growth inhibition, tumor eradication, and/or T cell activating properties as e.g. determined in vitro), or binds MHC-displayed NTDNNLAVY (SEQ ID NO: 54) with better affinity. In certain embodiments, the antigen binding protein of the disclosure is more specific than a competitor protein for MHC-displayed NTDNNLAVY (SEQ ID NO: 54) and shows a better off-target profile. In certain embodiments, the antigen binding protein shows less binding to one or more of SEQ ID NO: 55, SEQ ID NO: 56, SEQ ID NO: 57, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75, compared to the competitor molecule. In certain embodiments, the antigen binding protein shows less binding to NSDSNLTTY (SEQ ID NO: 61) and/or TADHNLLLY (SEQ ID NO: 62), compared to the competitor molecule.

### Chimeric Antigen Receptors

In one aspect, the disclosure provides chimeric antigen receptors (CARs) and immune cells engineered to express such CARs, comprising the antigen binding proteins described herein, in particular the monovalent pMHC binding proteins.

As used herein, the term “chimeric antigen receptor” or “CAR” refers to a receptor that is capable of activating an immune cell in response to antigen binding. CARs are recombinant membrane spanning molecules and are advantageously expressed on immune cells. Their structure typically comprises (i) an extracellular domain (ectodomain or antibody domain), (ii) a transmembrane domain and (iii) a cytoplasmic domain (endodomain or intracellular signaling domain).

The ectodomain (i.e., antibody domain) typically comprises a scFv but other antigen binding protein formats may also be used. A spacer connects the ectodomain and the transmembrane domain, which in turn is connected to an endodomain. Upon binding of the ectodomain to the antigen, the receptors cluster and an activation signal is transmitted to the cell which results in initiation of an immune response. First generation CARs have a simply structured endodomain comprising CD3-zeta. To increase the activation signal, a co-stimulatory domain was added in the second-generation CARs; and third generation CARs include two or more co-stimulatory domains (Maus M V et al (2014) Blood, 123:2625-2635). Said co-stimulatory domains may be selected from the group consisting of CD28, OX40 and/or 4-1BB. Apart from CD3-zeta, other ITAM-containing domains have been explored including the Fc receptor for IgE-γ domain.

Suitable immune cells include, without being limited to, T cells, Natural Killer T (NKT) cells, natural killer (NK) cells, human embryonic stem cells, hematopoietic stem cells (HSC) or induced pluripotent stem cells (iPS). Such T cell may be a cytotoxic T lymphocyte (CTL), a regulatory T lymphocyte, an inflammatory T-lymphocytes, or a helper T-lymphocyte or a gamma-delta T cell. The T cell may be a CD4+ or CD8+ or a mixed population of CD4+ and CD8+ cells.

In one aspect, the disclosure provides a chimeric antigen receptor (CAR) that specifically recognizes a peptide-MHC, comprising: i) an antigen binding protein with specificity to the peptide-MHC; ii) a transmembrane domain; and iii) an intracellular signaling domain, wherein the peptide-MHC is MHC-displayed NTDNNLAVY (SEQ ID NO: 54).

In certain embodiments, the CAR comprises a scFv comprising the amino acid sequence of SEQ ID NO: 33, or a variant thereof that is at least about 90%, 95%, 96%, 97%, 98% or 99% identical thereto.

In certain embodiments, the transmembrane domain is selected from the group consisting of an artificial hydrophobic sequence and transmembrane domains of a type I transmembrane protein, an alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

In certain embodiments, the intracellular signaling domain is selected from the group consisting of cytoplasmic signaling domains of a human CD3 zeta chain, FcyRIII, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

The antibody domain may be any of the antigen binding proteins outlined above. Thus, in certain embodiments, the antibody domain comprises an antibody variable light domain (VL) comprising an amino acid sequence represented by the formula LFR1-CDRL1-LFR2-CDRL2-LFR3-CDRL3-LFR4. In certain embodiments, the antibody domain comprises an antibody variable heavy domain (VH) comprising an amino acid sequence represented by the formula HFR1-CDRH1-HFR2-CDRH2-HFR3-CDRH3-HFR4. In certain embodiments, the antibody domain comprises an scFv as described elsewhere herein.

### Reduction of Anti-Drug Antibody Binding

Anti-drug antibodies (ADAs) may affect the risk profile and efficacy of a biological drug. If neutralizing, they may block the drug's ability to bind to its target. It is therefore a regulatory requirement to test biologic drugs for the binding of anti-drug antibodies and their neutralizing potential. Anti-drug antibody assays are e.g., detailed in WO2007101661A1 (Hoffmann La Roche), WO2018178307A1 (Ablynx), WO2021046316A2 (Adverum Biotechnologies, Charles River), and US20180088140A1 (Genzyme Corporation), each of which is incorporated herein by reference.

Anti-drug antibodies binding to a tumor targeting domain of an antigen binding protein may lead to clustering of said antigen binding protein when each variable domain of the ADA binds to one tumor targeting domain of two antigen binding proteins. The two or more CD3 binding domains on said antigen binding protein cluster and overstimulate the targeted T cell in the absence of target engagement, thereby leading to off-target toxicity. Unspecific stimulation of the T-cells may lead to systemic cytokine release.

Generally, there is a need in the art to develop safer and more effective bispecific antibodies for cancer immunotherapy.

The inventors have found that certain mutations in the tumor antigen binding domain of a T cell engager reduce ADA response and at the same time reduce nonspecific T cell stimulation in the absence of target engagement. Thereby, a highly effective and safe approach for cancer immunotherapy is provided.

For such purpose, a variable heavy chain amino acid at position 11, 89, and/or 108, according to Kabat numbering, is substituted with a polar amino acid; and/or serine(S) at position 113 is deleted, according to Kabat numbering. Such substitution is particularly favorable when the binding domain is in scFv format. In case of a Fab-scFv₂, one or both scFvs may comprise such substitution or deletion.

In certain embodiments, the polar amino acid is serine(S) and/or threonine (T).

In certain embodiments, the heavy chain amino acid is substituted with serine(S) at heavy chain amino acid position 11, serine(S) or threonine (T) at heavy chain amino acid position 89, and/or serine(S) or threonine (T) at heavy chain amino acid position 108, according to Kabat numbering.

In certain embodiments, the heavy chain amino acid is substituted with serine(S) at heavy chain amino acid position 11, serine(S) at heavy chain amino acid position 89, and serine(S) at heavy chain amino acid position 108, according to Kabat numbering.

### Expression of Antigen Binding Proteins

In one aspect, polynucleotides or nucleic acids encoding the antigen binding proteins (including the multispecific antigen binding proteins) disclosed herein are provided, such as isolated polynucleotides or nucleic acids which are typically synthetic. Methods of making an antigen binding protein expressing these polynucleotides or nucleic acids are also provided.

Polynucleotides encoding the antigen binding proteins disclosed herein are typically inserted in a cloning vector or into an expression vector, e.g., in form of cDNA, for introduction into host cells that may be used to produce the desired quantity of the antigen binding proteins. Accordingly, in certain aspects, the invention provides expression vectors comprising the polynucleotides disclosed herein and host cells comprising these vectors and polynucleotides.

The term “vector” or “expression vector” is used herein to mean vectors used in accordance with the present invention as a vehicle for introducing into and expressing a desired gene (e.g., polynucleotides or nucleic acids encoding the antigen binding proteins disclosed herein) in a cell. A vector may be a self-replicating nucleic acid structure or incorporate into the genome upon introduction into the host cell. As known to those skilled in the art, such vectors may readily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (e.g., RSV, MMTV, MOMLV or the like), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the antigen binding proteins may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding an antigen binding protein such as an antibody, or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, DG44 and DUXB11 (Chinese hamster ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney) and the like. In one embodiment, the cell line provides for altered glycosylation, e.g., afucosylation, of the antibody expressed therefrom (e.g., PER.C6® (Crucell) or FUT8-knock-out CHO cell lines (Potelligent® cells) (Biowa, Princeton, N.J.)). Host cell lines are typically available from commercial services, e.g., the American Tissue Culture Collection, or from published literature. In one embodiment, the host cell is not a cell within a human body.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the antigen binding proteins featured in the invention can also be expressed in non-mammalian cells such as bacteria or yeast or plant cells. In this regard it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the proteins can become part of inclusion bodies. The proteins must be isolated, purified and then assembled into functional molecules.

In addition to prokaryotes, eukaryotic microbes may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRPI gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

In another aspect, the disclosure provides a nucleic acid encoding the antigen binding proteins recited above, such as the bispecific antigen binding protein recited above.

In another aspect, the disclosure provides an expression vector comprising the nucleic acid recited above.

In another aspect, the disclosure provides a host cell comprising the expression vector recited above.

In another aspect, the disclosure provides a method of manufacturing the antigen binding proteins recited above, such as the bispecific antigen binding protein recited above, comprising the steps of: (i) cultivating the host cell recited above under conditions allowing expression of the antigen binding proteins; subsequently (ii) recovering the antigen binding proteins; and optionally (iii) further purifying and/or modifying and/or formulating the antigen binding proteins.

In certain embodiments, a method of manufacturing a bispecific antigen binding protein es described above is provided, comprising the steps of: (i) cultivating the host cell recited above under conditions allowing expression of the bispecific antigen binding protein; followed by (ii) recovering the bispecific antigen binding protein; and optionally (iii) further purifying and/or modifying and/or formulating the bispecific antigen binding protein.

In certain embodiments, a method of manufacturing a CAR-expressing cell as described above is provided, comprising the steps of: (i) introducing a nucleic acid encoding the CAR into the cell to produce the CAR-expressing cell; and optionally followed by (ii) isolating the CAR-expressing cell.

### Engineering and Optimization of Antigen Binding Proteins

The antigen binding proteins, including the multispecific antigen binding proteins, of the disclosure may be engineered or optimized. As used herein, “optimized” or “optimization” refers to the alteration of an antigen binding protein to improve one or more functional properties. Alteration includes, but is not limited to, deletions, substitutions, additions, and/or modifications of one or more amino acids within an antigen binding protein.

As used herein, the term “functional property” is a property of an antigen binding protein for which an improvement (e.g., relative to a conventional antigen binding protein, such as an antibody) is desirable and/or advantageous to one of skill in the art, e.g., in order to improve the manufacturing properties or therapeutic efficacy of an antigen binding protein. In one embodiment, the functional property is stability (e.g., thermal stability). In another embodiment, the functional property is solubility (e.g., under cellular conditions). In yet another embodiment, the functional property is aggregation behavior. In still another embodiment, the functional property is protein expression (e.g., in a prokaryotic cell). In yet another embodiment the functional property is refolding behavior following inclusion body solubilization in a manufacturing process. In certain embodiments, the functional property is not an improvement in antigen affinity. In another embodiment, the improvement of one or more functional properties has no substantial effect on the affinity of the antigen binding protein.

Alterations, such as deletions, substitutions, and/or insertions, can be introduced into parental sequences by a variety of standard techniques known in the art, such as combinatorial chemistry, site-directed DNA mutagenesis, PCR-mediated and/or cassette mutagenesis, peptide/protein chemical synthesis, chemical reaction specifically modifying reactive groups in the parental binding member. The variants so formed can be tested by routine methods for their chemical, biological, biophysical and/or biochemical properties, e.g., by the methods described elsewhere herein.

In certain embodiments, the substitution is a conservative amino acid substitution. As used herein, the term “conservative substitution” refers to replacing an amino acid with a replacement amino acid that is physically, biologically, chemically and/or functionally similar to the replacement amino acid, e.g., has a similar size, shape, electric charge and/or chemical properties, including the ability to form covalent or hydrogen bonds. Non-conservative substitutions, in contrast, may lead to substantial changes, e.g., with respect to the charge, dipole moment, size, hydrophilicity, hydrophobicity or conformation of the antigen binding protein.

In certain embodiments, the antigen binding protein of the disclosure is an scFv and is optimized by identifying preferred amino acid residues to be substituted, deleted, and/or added at amino acid positions of interest (e.g., amino acid positions identified by comparing a database of scFv sequences having at least one desirable property, e.g., as selected with Quality Control (QC) assay, versus a database of mature antibody sequences, e.g., the Kabat database) in an antigen binding protein. Thus, the disclosure further provides “enrichment/exclusion” methods for selecting a particular amino acid residue. Still further, the disclosure provides methods of engineering antigen binding proteins (e.g., scFvs) by mutating particular framework amino acid positions identified using the “functional consensus” approach described herein. In certain embodiments, the framework amino acid positions are mutated by substituting the existing amino acid residue by a residue which is found to be an “enriched” residue using the “enrichment/exclusion” analysis methods described herein. In one aspect, the disclosure provides a method of identifying an amino acid position for mutation in a single chain antibody (scFv), the scFv having VH and VL amino acid sequences, the method comprising: a) entering the scFv VH, VL or VH and VL amino acid sequences into a database that comprises a multiplicity of antibody VH, VL or VH and VL amino acid sequences such that the scFv VH, VL or VH and VL amino acid sequences are aligned with the antibody VH, VL or VH and VL amino acid sequences of the database; b) comparing an amino acid position within the scFv VH or VL amino acid sequence with a corresponding position within the antibody VH or VL amino acid sequences of the database; c) determining whether the amino acid position within the scFv VH or VL amino acid sequence is occupied by an amino acid residue that is conserved at the corresponding position within the antibody VH or VL amino acid sequences of the database; and d) identifying the amino acid position within the scFv VH or VL amino acid sequence as an amino acid position for mutation when the amino acid position is occupied by an amino acid residue that is not conserved at the corresponding position within the antibody VH or VL amino acid sequences of the database. ScFv optimization is described in further detail in WO2008110348, WO2009000099, WO2009000098, and WO2009155725, all of which are incorporated herein by reference.

In those aspects of the disclosure where the presence of an Fc domain is practicable, the antigen binding protein may comprise an Fc domain which is modified such that it does not induce cytotoxic immune responses and/or does not activate complement. For example, one or more substitutions may be introduced into the Fc domain so that its ADCC/ADCP or CDC effector function is inactivated. Such antigen binding protein has the advantage of increased half-life when compared to antibody fragments with a molecular weight below 60 kDa, without mediating cytotoxic immune responses.

Certain mutations in the antigen binding domains of a T cell engager were found to reduce ADA response and at the same time reduce nonspecific T cell stimulation in the absence of target engagement. Thus, the antigen binding protein of the disclosure, in particular when in the scFv format, may comprise a variable heavy chain having a non-polar amino acid at position 11, 89 and/or 108, according to Kabat numbering. For example, the variable heavy chain comprises: leucine (L) or serine(S) at amino acid position 11, according to Kabat numbering; valine (V), serine(S), or threonine (T) at amino acid position 89, according to Kabat numbering; and/or leucine (L), serine(S), or threonine (T) amino acid position 108, according to Kabat numbering.

Chemical and/or Biological Modifications

After their biosynthesis, the proteins disclosed herein (e.g., antigen binding proteins and multispecific antigen binding proteins described herein) may undergo a variety of modifications, such as Post-Translational Modifications (PTMs), either through natural processes or synthetically driven.

Hence, in one aspect, the antigen binding protein (such as the multispecific antigen binding protein described above) is chemically and/or biologically modified compared to the reference sequence given herein. For example, the antigen binding protein may be glycosylated, phosphorylated, hydroxylated, PEGylated, HESylated, PASylated, XTENylated, sulfated, labeled with dyes and/or radioisotopes, conjugated with enzymes and/or toxins, and/or Albumin binding or fusion technology. Likewise, any nucleic acid sequence, plasmid or vector and/or host cell described herein may be modified accordingly.

Such modification may for example be done to optimize pharmacokinetics, the water solubility or to lower side effects. For example, PEGylation, PASylation, XTENylation, HESylation and/or the fusion to serum albumin may be applied to slow down renal clearance, thereby increasing plasma half-life time of the antigen binding protein. In ne embodiment, the antigen binding molecules of the disclosure are operably linked to human serum albumin. In one embodiment, a modification adds a different functionality to the antigen binding protein, for example, a detection label for diagnostics or a toxin to combat cancer cells even more efficiently.

Alternatively, or additionally, in some embodiments, the antigen binding proteins and other polypeptides provided herein undergo co- and post-translational modifications as known in the art. Examples of post-translational modifications include, but are not limited to, disulfide bond formation, glycosylation, cyclization (such as e.g., N-terminal pyroglutamate formation), have a N-terminal or C-terminal residue removed or “clipped” (for example, C-terminal lysine residues are often removed during the manufacturing process), deamidation, isomerization, oxidation, glycation, acylation, fucosylation, peptide bond cleavage, non-reductible cross-linking, truncation, and/or have part or all of a signal sequence incompletely processed.

In certain embodiments, the CD3 antigen binding domain comprises an N-terminal truncation of 1 or more amino acids (e.g., a N-terminal truncation of 1, 2, 3, 4, or 5 amino acids). In certain embodiments, the CD3 antigen binding domain comprises a C-terminal truncation of 1 or more amino acids (e.g., a C-terminal truncation of 1, 2, 3, 4, or 5 amino acids). In certain embodiments, the N-terminal and/or C-terminal truncation is a truncation of the Fab-(scFv)₂ of a heavy chain amino acid sequence of SEQ ID NO: 52 and a light chain amino acid sequence of SEQ ID NO: 53. In certain embodiments, the N-terminal and/or C-terminal truncation is a truncation of the Fab-(scFv) comprising a heavy chain amino acid sequence of SEQ ID NO: 34 and a light chain amino acid sequence of SEQ ID NO: 35. In certain embodiments, the Q at position 1 of the antigen binding proteins is removed which may prevent pyroglutamate formation and protects the protein to be clipped by proteases; in certain embodiments, this may lower the possibility of having different charge variants which may result challenging in the CMC process. An N-terminal R removal may in certain embodiments result in a lower pre-existing antibody response.

In certain embodiments, the variant sequence of SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 52 and/or SEQ ID NO: 53 comprises a pyroglutamate at amino acid position 1. In certain embodiments, the light chain of the antigen binding protein comprises pyroglutamate (pE) instead of the N-terminal glutamine.

In one embodiment, the antigen binding protein is glycosylated. Glycosylation refers to a process that attaches carbohydrates to proteins. In biological systems, this process is performed enzymatically within the cell as a form of co-translational and/or post-translational modification. A protein can also be chemically glycosylated. The carbohydrates may be N-linked to a nitrogen of asparagine or arginine side-chains; O-linked to the hydroxy oxygen of serine, threonine, tyrosine, hydroxylysine, or hydroxyproline side-chains; employ xylose, fucose, mannose, and N-acetylglucosamine attached to a phospho-serine; and/or adding mannose sugar to a tryptophan residue found in a specific recognition sequence. Glycosylation patterns may, e.g., be controlled by choosing appropriate cell lines, culturing media, protein engineering manufacturing modes and process strategies (see., HOSSLER, P. Optimal and consistent protein glycosylation in mammalian cell culture. Glycobiology 2009, vol. 19, no. 9, p. 936-949.). In some embodiments, the glycosylation patterns of the antigen binding proteins described herein are modified to enhance ADCC and CDC effector function.

The antigen binding protein may be engineered to control or alter the glycosylation pattern, e.g., by deleting and/or adding of one or more glycosylation sites. The creation of glycosylation sites can e.g., be accomplished by introducing the corresponding enzymatic recognition sequence into the amino acid sequence of the antigen binding protein.

In certain embodiments, the (multispecific) antigen binding protein comprises a pyroglutamate (pE, pyrGlu, pyre or pGlu) instead of the N-terminal glutamine or the N-terminal glutamate. In certain embodiments, the light chain of the antigen binding protein comprises pyroglutamate (pE) instead of the N-terminal glutamine. In certain embodiments, the light chain of the antigen binding protein comprises pyroglutamate (pE) instead of an N-terminal glutamate. In certain embodiments, such pyroglutamate (pE) modification has no impact on the safety and/or efficacy of the (multispecific) antigen binding protein. In certain embodiments, the N-terminal glutamine (Q) of a light chain disclosed herein is clipped, e.g., to prevent pyroglutamate formation.

In some embodiments, the antigen binding protein is PEGylated. PEGylation may alter the pharmacodynamic and pharmacokinetic properties of a protein. Additionally, PEGylation may reduce the immunogenicity by shielding the PEGylated antigen binding protein from the immune system and/or alter its pharmacokinetics by, e.g., increasing the in vivo stability of the antigen binding protein, protecting it from proteolytic degradation, extending its half-life time and by altering its biodistribution. Typically, polyethylene-glycol (PEG) of an appropriate molecular weight is covalently attached to the protein. Similar effects may be achieved using PEG mimetics, e.g., HESylating, PASylating, or XTENylating the antigen binding protein. HESylation utilizes hydroxyethyl starch (“HES”) derivatives. During PASylation, the antigen binding protein is linked to conformationally disordered polypeptide sequences composed of the amino acids proline (P), alanine (A) and serine(S), and XTENylation employs a similar, intrinsically disordered XTEN-polypeptide.

In certain embodiments, the antigen binding protein (e.g., the multispecific antigen binding protein) is linked to or combined with a functional entity such as a detectable label, a therapeutic agent or a PK modifying moiety. For example, the antigen binding protein is labelled with or conjugated to an entity which attributes one or more ancillary functions to the antigen binding protein. For example, the entity may have an additional immunological effector function, be effective in drug targeting or useful for detection. The functional entity can, e.g., be chemically linked or fused genetically to the antigen binding protein using known methods in the art. As used herein, the term “label” refers to any substance or ion which is indicative of the presence of the antigen binding protein when detected or measured by physical or chemical means, either directly or indirectly. For example, the label may be directly detectable by, without being limited to, light absorbance, fluorescence, reflectivity, light scatter, phosphorescence, or luminescence properties, molecules or ions detectable by their radioactive properties or molecules or ions detectable by their nuclear magnetic resonance or paramagnetic properties. Examples of indirect detection include light absorbance or fluorescence; for example, various enzymes which cause appropriate substrates to convert, e.g., from non-light absorbing to light absorbing molecules, or from non-fluorescent to fluorescent molecules. A labelled antigen binding protein is particularly useful for in vitro and in vivo detection or diagnostic purposes. For example, an antigen binding protein labelled with a suitable radioisotope, enzyme, fluorophore or chromophore can be detected by radioimmunoassay (RIA), enzyme-linked immunosorbent assay (ELISA), or flow cytometry-based single cell analysis (e.g., FACS analysis), respectively. Similarly, the nucleic acids and/or vectors disclosed herein can be labeled for detection or diagnostic purposes, e.g., using labelled fragments thereof as probes in hybridization assays.

Non-limiting examples of functional entities include radioisotopes, apoenzymes, enzymes (e.g., alkaline phosphatase, horseradish peroxidase, beta-galactosidase and/or angiogenin), co-factors, peptide moieties (e.g., a HIS-tag), proteins (e.g. lectin, serum albumin), carbohydrates (e.g., mannose-6-phosphate tags), fluorophores (e.g., fluorescein isothiocyanate (FITC)), phycoerythrin, green/blue/red or other fluorescent proteins, allophycocyanin (APC), chromophores, vitamins (e.g., biotin), chelators, antimetabolites (e.g., methotrexate), toxins (e.g. a cytotoxic drug, or a radiotoxin).

In certain embodiments, the antigen-binding protein described herein (e.g., an antibody fragment such as an scFv, Fab, diabody, or other engineered binding domain), is conjugated to a detectable or therapeutic radioisotope. The radioisotope may be directly attached to the antigen-binding protein or indirectly linked through a chelator, linker, adaptor moiety, or other coupling chemistry suitable for stable association under physiological conditions. Representative radioisotopes include, without limitation, ¹²⁵I, ¹³¹I, ⁹⁰Y, ¹⁷⁷Lu, ⁶⁴Cu, ⁶⁷Cu, ²¹²Pb, ²¹²Bi, ²²⁵Ac, ¹⁸F, or any other radionuclide useful for diagnostic imaging, biodistribution analysis, target engagement assessment, or radioimmunotherapy.

The radioisotope-conjugated antigen-binding protein may be employed for in vitro, ex vivo, or in vivo applications, including but not limited to radioimmunoassay, positron emission tomography (PET), single-photon emission computed tomography (SPECT), autoradiography, intraoperative tumor detection, or targeted radiotherapeutic delivery to cells expressing the antigen of interest. In therapeutic embodiments, the conjugated radioisotope delivers ionizing radiation selectively to target cells, thereby inducing cytotoxicity while sparing non-target tissues to the extent permitted by antibody specificity, affinity, and pharmacokinetics. In one aspect, the invention relates to drug conjugates (in particular antibody-drug conjugates ADCs) comprising the antigen binding proteins described herein, e.g., a monovalent or a multispecific antigen binding protein described herein (e.g., an antibody), conjugated to a toxin which further enhances efficient killing of specific cells, such as e.g., MHC-displayed NTDNNLAVY (SEQ ID NO: 54) positive cells. The toxin moiety is typically a small molecular weight moiety, such as MMAE/MMAF, DM1, chaliceamicin, anthracycline toxins, taxol, gramicidin D and/or colchicine, which may be linked via a peptide linker to the antigen binding protein.

The toxin may be conjugated non-site-specifically or site-specifically to the antigen binding protein. Non-site-specific conjugation typically involves the use of chemical linkers, e.g., with maleimide functionality, that mediate conjugation to lysine or cysteine amino acid side chains of the antigen binding protein or to the amino-group of the N-terminus. Site-specific conjugation may be achieved using chemical, chemo-enzymatic, or enzymatic conjugations known in the art, e.g., employing bifunctional linkers, bacterial transglutaminase or sortase enzymes, linkers allowing Pictet-Spengler chemistry on formyl-glycine forming enzyme modified antigen binding proteins, or glycan-remodeled antigen binding proteins.

### Methods of Administering Antigen Binding Proteins

Methods of preparing and administering antigen binding proteins of the disclosure (such as the multispecific antigen binding protein described above) as well as the nucleic acids described herein, the vectors described herein, the host cells described herein or the compositions described herein to a subject are well known to or are readily determined by those skilled in the art. The route of administration of the antigen binding proteins of the current disclosure may e.g., be oral, parenteral, by inhalation, or topical. The term parenteral as used herein includes intravenous, intraarterial, intraperitoneal, intramuscular, subcutaneous, rectal or vaginal administration. The term topical as used herein includes, but is not limited to, administration with liquid or solution eye drops, emulsions (e.g., oil-in-water emulsions), suspensions, and ointments.

While all these forms of administration are clearly contemplated as being within the scope of the current disclosure, a form for administration would be a solution for injection. Usually, a suitable pharmaceutical composition for injection may comprise a buffer (e.g., acetate, phosphate or citrate buffer), a surfactant (e.g., polysorbate), optionally a stabilizer agent (e.g., human albumin), etc. However, in other methods compatible with the teachings herein, the modified antibodies can be delivered directly to the site of the adverse cellular population thereby increasing the exposure of the diseased tissue to the therapeutic agent.

Effective doses of the compositions of the present disclosure, for the treatment of the related conditions vary depending upon many different factors, including means of administration, target site, physiological state of the patient, whether the patient is human or an animal, other medications administered, and whether treatment is prophylactic or therapeutic. Usually, the patient is a human, but non-human mammals, including transgenic mammals, can also be treated. Treatment dosages may be titrated using routine methods known to those of skill in the art to optimize safety and efficacy.

As previously discussed, the antigen binding proteins of the present disclosure (e.g., the multispecific antigen binding protein), conjugates or recombinants thereof may be administered in a pharmaceutically effective amount for the in vivo treatment of mammalian disorders. In this regard, it will be appreciated that the disclosed antigen binding proteins will be formulated to facilitate administration and promote stability of the active agent. As used herein, an “effective amount” of an agent, e.g., a pharmaceutical composition, refers to an amount effective to achieve the desired therapeutic or prophylactic result, at dosages and for periods of time as necessary.

In another aspect, the disclosure provides for the use of the antigen binding protein recited above, such as the bispecific antigen binding protein recited above, for preparing a pharmaceutical composition for treating a KK-LC-1 associated cancer in a subject.

In another aspect, the disclosure provides a pharmaceutical composition comprising the antigen binding proteins recited above, in particular the bispecific antigen binding protein recited above, and a pharmaceutically acceptable carrier.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the antigen binding proteins (such as the multispecific antigen binding protein) shall be held to mean an amount sufficient to achieve effective binding to an antigen and to achieve a benefit, e.g., to ameliorate symptoms of a disease or disorder or to detect a substance or a cell. In the case of tumor cells, the antigen binding proteins will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the antigen binding proteins of the disclosure (such as the multispecific antigen binding protein described above) may be administered to a human or other animal in accordance with the aforementioned methods of treatment in an amount sufficient to produce a therapeutic or prophylactic effect. The antigen binding proteins of the disclosure can be administered to such human or other animal in a conventional dosage form prepared by combining the antigen binding proteins of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of antigen binding proteins described in the current disclosure may prove to be particularly effective. Similarly, the nucleic acids described herein, the vectors described herein, the host cell cells described herein (in particular the immune cells bearing a CAR) or the compositions described herein may be administered to a human or other animal in accordance with the methods of treatment described above in an amount sufficient to produce a therapeutic or prophylactic effect.

“Efficacy” or “in vivo efficacy” as used herein refers to the response to a therapy by the pharmaceutical composition of the disclosure, using e.g., standardized response criteria. The success or in vivo efficacy of the therapy using a pharmaceutical composition of the disclosure refers to the effectiveness of the composition for its intended purpose, i.e., the ability of the composition to cause its desired effect. The in vivo efficacy may be monitored by established standard methods for the specific diseases. In addition, various disease specific clinical chemistry parameters and other established standard methods may be used.

In some embodiments, the compounds and cells described herein are administered in combination with one or more different pharmaceutical compounds. Generally, therapeutic use of the compounds and cells described herein may be in combination with one or more therapies selected from the group of antibody therapy, chemotherapy, cytokine therapy, dendritic cell therapy, gene therapy, hormone therapy, laser light therapy, radiation therapy or vaccine therapy.

### Methods of Treating Cancer

Provided herein are methods of treating cancer with the antigen binding proteins and in particular with the multispecific antigen binding proteins of the disclosure (e.g., an antigen binding protein comprising a Fab domain which binds a cell surface protein of an immune cell linked to a first and second pMHC antigen binding protein). The methods may be used to treat patients having any tumor type in which at least some of the cancer cells express a target peptide as disclosed herein displayed on a pMHC, such as MHC-displayed NTDNNLAVY (SEQ ID NO: 54). Such target peptide positive cancers or cancer cells can be assessed using any method known in the art, including, but are not limited to, detecting RNA expression levels or histological methods such as Immunohistochemistry (IHC).

In certain embodiments of the antigen binding protein of the disclosure, the target pMHC binding domain specifically targets an MHC restricted peptide derived of a tumor antigen.

In some embodiments, the target peptide positive cancer is a solid tumor and/or a hematological tumor, optionally wherein the cancer is selected from esophageal cancer, gastric adenocarcinoma, lung adenocarcinoma, and lung squamous cancer.

In one aspect, the upregulation of MHC-displayed NTDNNLAVY (SEQ ID NO: 54) expression on the cell surface is a biomarker for cancer.

In one aspect, the disclosure provides a method for killing a target cell comprising a major histocompatibility complex (MHC) presenting a neoantigen, the method comprising: a) contacting a plurality of cells comprising immune cells and the target cell with the antigen binding protein described herein, such as the multispecific antigen binding protein above, wherein said antigen binding protein specifically binds to the pMHC on the surface of the target cell and to CD3 on the surface of the immune cells; b) forming a specific binding complex through the antigen binding protein interactions with the target cells and the immune cells, thereby activating the immune cells; and c) killing the target cell with the activated immune cells.

In one aspect, the disclosure provides a method of treating cancer comprising the step of administering the antigen binding protein described herein, the nucleic acid described herein, vectors described herein, the host cells described herein or the pharmaceutical composition described herein, to a patient in need thereof.

In one aspect, the aforementioned antigen binding proteins (including antibody-drug conjugates), nucleic acids, vectors or host cells (in particular immune cells expressing CARs) or the vector, are useful as a medicament. Typically, such a medicament includes a therapeutically effective amount of a molecule or cell as provided herein. Accordingly, a respective molecule or host cell can be used for the production of a medicament useful in the treatment of one or more disorders, in particular disorders or diseases consisting of MHC-displayed NTDNNLAVY (SEQ ID NO: 54).

Also provided is the use of the aforementioned antigen binding proteins, nucleic acids, vectors or host cells (in particular immune cells expressing CARs) in the manufacture of a medicament.

In one aspect, a method of treating disorder or diseases consisting of MHC-displayed NTDNNLAVY (SEQ ID NO: 54) is provided. The method includes the steps of administering a pharmaceutically effective amount of a molecule or host cell as described herein, in particular the antigen binding proteins or a CAR expressing cell, to a subject in need thereof. In one embodiment, the pharmaceutical composition described above, which includes such pharmaceutically effective amount of the antigen binding protein, nucleic acid, vector or host cell (e.g., immune cell) is administered to the subject. The medicament referred to above may be administered to a subject.

In another aspect, the disclosure provides a method of treating a MHC-displayed NTDNNLAVY (SEQ ID NO: 54) positive cancers, in a patient in need thereof comprising administering to the patient a therapeutically effective amount of the antigen binding protein described herein, such as the multispecific antigen binding protein described herein, the CAR described herein, the host cell (e.g., immune cell) described herein, or the pharmaceutical composition recited above.

In certain aspects, patients eligible for treatment with an antagonist as described herein are selected based on RNA sequencing and/or immunohistochemistry (IHC), such as for detection of total target peptide.

The subject in need of treatment can be a human or a non-human animal. In typical embodiments, the subject is diagnosed with an MHC-displayed NTDNNLAVY (SEQ ID NO: 54) related disorder or may acquire such a disorder. In case of an animal model, the animal might be genetically engineered to develop MHC-displayed NTDNNLAVY (SEQ ID NO: 54) related disorder. An animal may also be genetically engineered in such a way that it shows the characteristics of MHC-displayed NTDNNLAVY (SEQ ID NO: 54) related disease.

In certain embodiments, disorders or diseases consisting of MHC-displayed NTDNNLAVY (SEQ ID NO: 54) expression is cancer. In certain embodiments, the cancer is a solid tumor and/or a hematological tumor, optionally wherein the cancer is selected from esophageal cancer, gastric adenocarcinoma, lung adenocarcinoma, and lung squamous cancer. All the technical features described in the present disclosure regarding the antigen binding proteins are applicable.

### Use in Diagnostics and Detection Assays

An antigen binding protein as disclosed herein may be used for detection or diagnostic purposes in vivo and/or in vitro. For example, a wide range of immunoassays using antibodies for detecting the expression in specific cells or tissues are known to the skilled person. For such purposes, it may be advantageous to use an antigen binding protein connected to a detectable label, such as biotin.

In one embodiment, the described antigen binding proteins are useful for detecting the presence of a target peptide-MHC complex as described elsewhere herein in a sample. The detection may be for quantitative or qualitative purposes. The sample is preferably of biological origin, such as blood, urine, cerebrospinal fluid, biopsy, lymph and/or non-blood tissues. In certain embodiments, a biological sample comprises a cell or tissue from a human patient. In certain embodiments, the method includes contacting a biological sample with an antigen binding protein described herein, the CAR described herein, the immune cell described herein, the under conditions permissive for binding of the inhibitor to the target peptide-MHC and then detecting the inhibitor-target peptide-MHC and then detecting the inhibitor-target complex. Such method may be an in vitro or in vivo method. In some embodiments, such method is performed to select subjects eligible for therapy with the antigen binding protein described herein.

In one embodiment, the described antigen binding proteins are useful for detecting the presence of a target peptide-MHC complex as described elsewhere herein. Thus, in one aspect, a method is provided, comprising the steps of:
(i) providing a sample from a patient;
(ii) adding a target peptide detection antibody (e.g., the antigen binding protein described herein) to the sample; (iii) incubating said detection antibody and the sample; (iv) detecting said detection antibody bound to the sample; and (v) selecting the patient for treatment with a antagonist as disclosed herein if said detection antibody is bound by the sample.

### Kits

Also contemplated are kits comprising at least one nucleic acid library or antigen binding protein, such as an antibody, including the multispecific antigen binding protein, or the pharmaceutical composition as described herein, typically together with a packaged combination of reagents with instructions. In one embodiment, the kit includes a composition containing an effective amount of said antigen binding protein in unit dosage form. Such kit may comprise a sterile container comprising the composition; non-limiting examples of such containers include, without being limited to, vials, ampoules, bottles, tubes, syringes, blister-packs. In some embodiments, the composition is a pharmaceutical composition and the containers are made of a material suitable for holding medicaments. In one embodiment, the kit may comprise in a first container the antigen binding protein in lyophilized form and a second container with a diluent (e.g., sterile water) for reconstitution or dilution of the antigen binding protein. In some embodiments, said diluent is a pharmaceutically acceptable diluent. In one embodiment, the kit is for diagnostic purposes and the antigen binding protein is formulated for diagnostic applications. In one embodiment, the kit is for therapeutic purposes and the antigen binding protein is formulated for therapeutic applications.

Typically, the kit will further comprise a separate sheet, pamphlet or card supplied in or with the container with instructions for use. If the kit is intended for pharmaceutical use, it may further comprise one or more of the following: information for administering the composition to a subject having a related disease or disorder and a dosage schedule, description of the therapeutic agent, precautions, warnings, indications, counter-indications, overdosage information and/or adverse reactions.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1—Identification of Tumor-Associated KK-LC-1 Peptide Presented On HLA-A*01:01-Positive Cancer Tissues

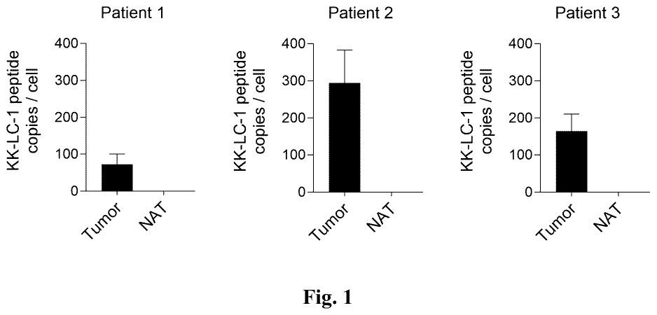

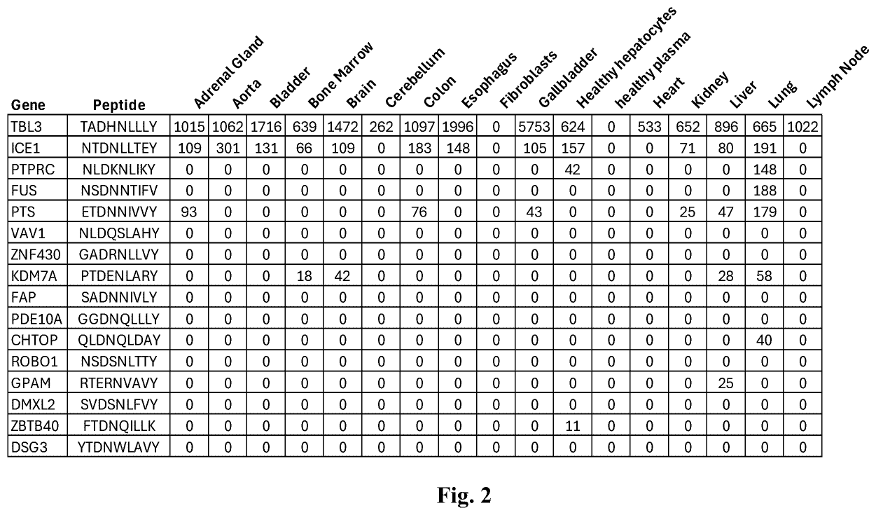

HLA-A*01:01 positive tumor samples from non-small cell lung cancer (NSCLC) patients were used for immunoprecipitation using the pan-HLA (W6/32) antibody. Peptides eluted from HLA complexes were processed for mass spectrometry analysis. Heavy-labelled KK-LC-1 peptide was spiked into the samples to allow quantification based on the established calibration curve. Copy number of KK-LC-1 peptide per cell was calculated based on tissue weight. Three out of six tumor biopsy samples were positive for KK-LC-1 target peptide NTDNNLAVY (SEQ ID NO: 54) with up to 400 copies per cell identified (FIG. 1).

### Example 2—Identification of Healthy Tissue-Displayed Off-Target Peptides Posing Cross-Reactivity Risk

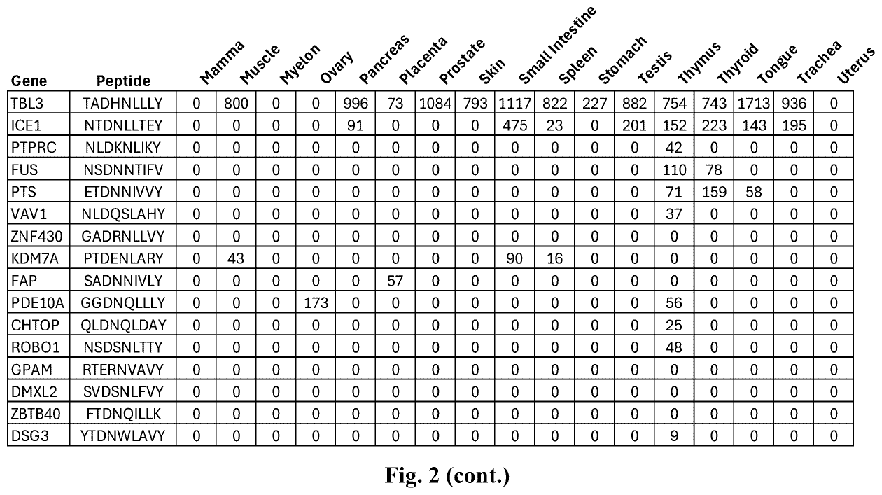

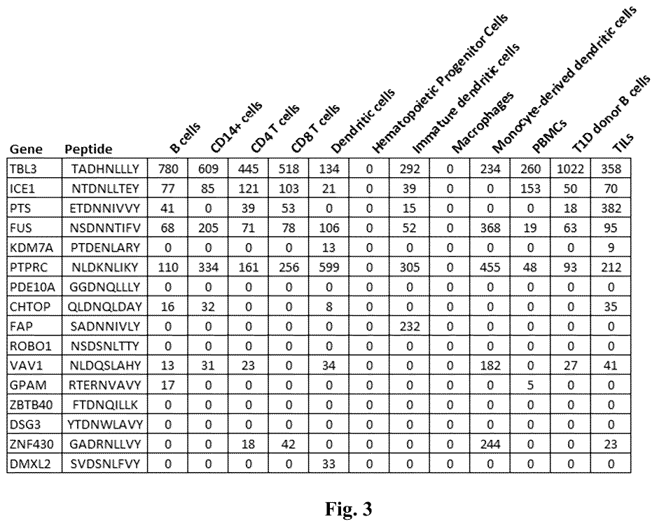

To identify off-target peptides posing a cross-reactivity risk in healthy tissues a Basic Local Alignment Search Tool (BLAST) search for peptides displaying high sequence identity to the KK-LC-1 target was performed using the full target peptide sequence NTDNNLAVY (SEQ ID NO: 54) as a query (https://blast.ncbi.nlm.nih.gov/Blast.cgi). Additionally, SCRAP database search (Yarmarkovich et al., 2023) was performed using the target sequence NTDNNLAVY (SEQ ID NO: 54) as a query dedicated to HLA-A*01:01. Peptide hits from different search tools were combined and sent for analysis to a normal tissue immunopeptidome database. Possible off-target peptides have been identified based on their high sequence similarity to the KK-LC-1 peptide, expression profile on healthy tissue and immune cells are listed in Table 3. FIG. 2 and FIG. 3 show the expression levels of peptides listed in Table 3 in healthy tissues and immune cells, respectively.

TABLE 3
List of endogenous physiologically relevant
peptides with high sequence similarity to KK-LC-1
NTDNNLAVY (SEQ ID NO: 54).

| Gene name | Sequence | Sequence ID |
|---|---|---|
| ICE1 | NTDNLLTEY | SEQ ID NO: 55 |
| PTS | ETDNNIVVY | SEQ ID NO: 56 |
| DSG3 | YTDNWLAVY | SEQ ID NO: 57 |
| KDM7A | PTDENLARY | SEQ ID NO: 58 |
| FUS | NSDNNTIFV | SEQ ID NO: 59 |
| PTPRC | NLDKNLIKY | SEQ ID NO: 60 |
| ROBO1 | NSDSNLTTY | SEQ ID NO: 61 |
| TBL3 | TADHNLLLY | SEQ ID NO: 62 |
| CHTOP | QLDNQLDAY | SEQ ID NO: 63 |
| DMXL2 | SVDSNLFVY | SEQ ID NO: 64 |
| FAP | SADNNIVLY | SEQ ID NO: 65 |
| GPAM | RTERNVAVY | SEQ ID NO: 66 |
| PDE10A | GGDNQLLLY | SEQ ID NO: 67 |
| VAV1 | NLDQSLAHY | SEQ ID NO: 68 |
| ZBTB40 | FTDNQILLK | SEQ ID NO: 69 |
| ZNF430 | GADRNLLVY | SEQ ID NO: 70 |

### Example 3—Production of HLA-A*01:01/KK-LC-1 Antigen for Animal Immunization

Genes of the MHC class I heavy chain, HLA-A*01:01 and B2m domain were each cloned into a pET-24D (+) vector using standard molecular biology techniques (J Biol Chem. 1995 Jan. 13; 270 (2): 971-7). E. coli BL-21 (DE3) cells were transformed with the expression vectors, cultivated and inclusion bodies were isolated. Refolding and purification of the solubilized HLA-A*01:01 extracellular domain (SEQ ID NO: 23), β2m (SEQ ID NO: 24) and KK-LC-1 peptide NTDNNLAVY (peptides & elephants, SEQ ID NO: 54) were performed, essentially as described by Rodenko et al. (2006). The purity of HLA-A*01:01/KK-LC-1 complex (pMHC) was assessed by SDS-PAGE and SE-HPLC. Amino acid sequences of each pMHC component are recited in Table 4.

TABLE 4
Amino acid sequences of HLA-A*01:01/KK-LC-1 antigen components

| Sequence ID | Sequence |
|---|---|
| HLA-A*01:01 | GSHSMRYFFTSVSRPGRGEPRFIAVGYVDDTQFVRFDSDA |
| extracellular | ASQKMEPRAPWIEQEGPEYWDQETRNMKAHSQTDRANL |
| domain | GTLRGYYNQSEDGSHTIQIMYGCDVGPDGRFLRGYRQDA |
| SEQ ID NO: 23 | YDGKDYIALNEDLRSWTAADMAAQITKRKWEAVHAAE |
| | QRRVYLEGRCVDGLRRYLENGKETLQRTDPPKTHMTHH |
| | PISDHEATLRCWALGFYPAEITLTWQRDGEDQTQDTELV |
| | ETRPAGDGTFQKWAAVVVPSGEEQRYTCHVQHEGLPKP |
| | LTLRWE |
| human β2m | IQRTPKIQVYSRHPAENGKSNFLNCYVSGFHPSDIEVDLLK |
| SEQ ID NO: 24 | NGERIEKVEHSDLSFSKDWSFYLLYYTEFTPTEKDEYACR |
| | VNHVTLSQPKIVKWDRDM |
| KK-LC-1 peptide | NTDNNLAVY |
| SEQ ID NO: 54 | |

### Example 4—Generation of Antibodies Against HLA-A*01:01/KK-LC-1

To generate specific antibodies that bind HLA-A*01:01/KK-LC-1 (SEQ ID NO: 54), New Zealand white rabbits were immunized with the recombinantly produced pMHC target complex. After confirmation of the presence of anti-HLA-A*01:01/KK-LC-1 (SEQ ID NO: 54) antibodies in the serum, the animals were sacrificed to extract their cervical lymph nodes and spleen.

Lymphocytes were isolated from lymph nodes and spleen of immunized animals to screen for B-cells that specifically recognize HLA-A*01:01/KK-LC-1 (SEQ ID NO: 54) using Fluorescence-activated cell sorting (FACS). Cells were deselected for binding to unrelated peptides on the HLA backbone and similar peptides loaded on HLA-A*01:01, see Table 5. Sorted B-cells were cultivated and secreted IgG from the B-cell supernatant was tested by ELISA for its binding to the target complex HLA-A*01:01/KK-LC-1 (SEQ ID NO: 54) and to similar peptides loaded on HLA-A*01:01. Signal ratio from the specific target binding to the unspecific binding was determined to identify hits binding specifically to the target.

TABLE 5
Similar and unrelated peptides to NTDNNLAVY (SEQ
ID NO: 54) loaded on HLA-A*01:01 used for
deselection of unspecific B-cells.

| | | Sequence |
|---|---|---|
| Gene name | Sequence | Identifier |
| PTS | ETDNNIVVY | SEQ ID NO: 56 |
| Synthetic peptide 1 | NTDEWRAVY | SEQ ID NO: 121 |
| Synthetic peptide 2 | NTDRWEAVY | SEQ ID NO: 122 |
| NUCG | NAWNNLEKY | SEQ ID NO: 123 |
| NP | CTELKLSDY | SEQ ID NO: 124 |
| Survivin | FTELTLGEF | SEQ ID NO: 125 |
| ORF1ab | DTDFVNEFY | SEQ ID NO: 76 |
| INO80E | LSSLASSRY | SEQ ID NO: 77 |
| VP11/12 | ATDSLNNEY | SEQ ID NO: 78 |
| OMD | NVDYGVPFY | SEQ ID NO: 79 |
| Hrp1 | ELARDSIYY | SEQ ID NO: 80 |
| DNMT3A R882H | YTDVSNMSH | SEQ ID NO: 81 |
| PXK | NSEEHSARY | SEQ ID NO: 82 |
| MAGE-A4 | EVDPTSNTY | SEQ ID NO: 83 |
| rev (HIV) | ISERILSTY | SEQ ID NO: 84 |
| E2 | QVDYYGLYY | SEQ ID NO: 85 |
| EZ030_09435 | YTTNDSSTAY | SEQ ID NO: 86 |
| p83/100 | NSNSSYYGKY | SEQ ID NO: 87 |
| KRAS.Q61L | ILDTAGLEEY | SEQ ID NO: 88 |
| MUC1 | SLEDPSTDYY | SEQ ID NO: 89 |
| ORF1ab | TTDPSFLGRY | SEQ ID NO: 90 |
| S | YTNSFTRGVY | SEQ ID NO: 91 |
| OmcB | VSDTENTHIY | SEQ ID NO: 92 |
| AIM-2 | RSDSGQQARY | SEQ ID NO: 93 |

To recover the sequences of the variable antibody domains of heavy (VH) and light chain (Vκ/Vλ) from the rabbit hits, RNA was extracted from the B-cells to generate cDNA via reverse transcription PCR. For each hit, a multiplex PCR with different primer sets was performed. PCR products were finally sequenced and reformatted as monovalent bispecific Fab-scFv molecules.

### Example 5—Production of pMHC-Targeting T Cell Engagers

Monovalent bispecific antigen binding proteins in Fab-scFv format were expressed by transient co-transfection in HEK293-6E cells. The antigen binding proteins were purified by an affinity chromatography from the supernatant. Size exclusion chromatography was performed as an additional purification step. Collected fractions were analyzed by SE-HPLC for monomer content and pooled accordingly. Final protein purity was assessed by SDS-PAGE and SE-HPLC.

### Example 6—Affinity Characterization of HLA-A*01:01/KK-LC-1×CD3 Antigen Binding Proteins

The hits in Fab-scFv format were evaluated for their ability to bind HLA-A*01:01/KK-LC-1 antigen by surface plasmon resonance (SPR) using a Biacore™ 8k (Cytiva) device. Briefly, 3000 RU of Biotin CAPture reagent (Cytiva) was immobilized on a Series S Sensor Chip CAP (Cytiva) and the biotinylated ligand HLA-A*01:01/KK-LC-1 (SEQ ID NO: 54) or control off-target peptide complexes with high sequence identity to KK-LC-1 peptide of SEQ ID NO: 54, i.e., HLA-A*01:01/DSG3 (SEQ ID NO: 57), HLA-A*01:01/PTS (SEQ ID NO: 56), HLA-A*01:01/KDM7A (SEQ ID NO: 58), and HLA-A*01:01/ICE1 (SEQ ID NO: 55) were diluted in HPS-EP+ running buffer (TEKNOVA) and captured for 300 s at a flow rate of 2 μL/min at flow cell 2 resulting in a ligand capture level of ≥100 RU. Subsequently, at least three consecutive analyte injections were performed at suitable concentrations applying the single-cycle-kinetics mode at a flow rate of 30 μL/min with an association time of 90 s and the dissociation time set to ≥400 s. The chip surface was regenerated according to the manufacturer's instructions. After reference flow cell signal and blank injections subtraction, the data was fit to a 1:1 Langmuir binding model to determine the association rate constant kₐ and dissociation rate constant k_{d} and calculate the equilibrium dissociation constant K_{D} (k_{d}/kₐ), also denoted as affinity, using the Biacore™ Insight Evaluation software.

Among all the hits, M2759 showed the most favorable binding profile with K_{D} of 84 nM to HLA-A*01:01/KK-LC-1 (SEQ ID NO: 54) and no binding to the control off-target peptide complexes with the highest sequence identity to HLA-A*01:01/KK-LC-1, i.e., HLA-A*01:01/DSG3 (SEQ ID NO: 57), HLA-A*01:01/PTS (SEQ ID NO: 56), HLA-A*01:01/KDM7A (SEQ ID NO: 58), and HLA-A*01:01/ICE1 (SEQ ID NO: 55).

### Example 7—Humanization of Rabbit-Derived Antigen Binding Proteins

The anti-KK-LC-1 scFv of M2759 was humanized using standard techniques known in the art (Tan et al. 2002, Lo 2004, Almagro et al. 2008, Williams et al. 2010, Safdari et al. 2013). Antibody fragment constructs in Fab-scFv format monovalent for CD3 and KK-LC-1 binding were generated, using a humanized sp34 variant. M2769 and M2883 comprised humanized variants of M2759 with varying number of rabbit-originating residues.

### Example 8—Characterization of Affinity and Binding Recognition Motif of Humanized HLA-A*01:01/KK-LC-1×CD3 Bispecific Antigen Binding Proteins

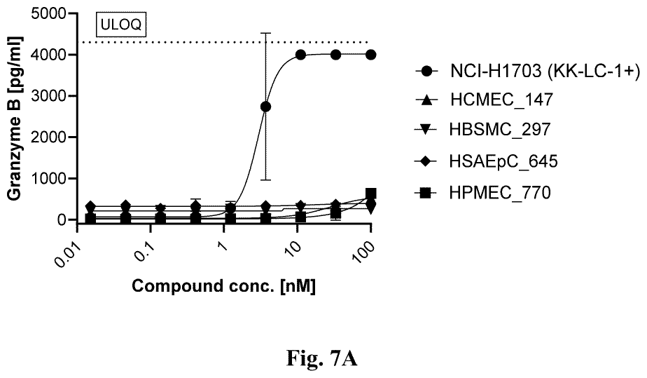

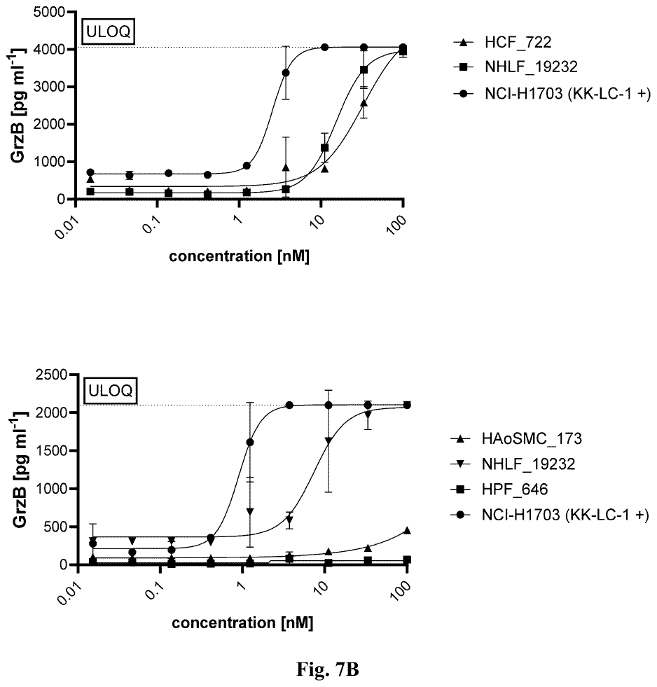

Humanized variants of M2759 were characterized for binding to the target HLA-A*01:01/KK-LC-1 (SEQ ID NO: 54) using SPR, as described in Example 6. Among the tested variants, M2883 showed the most favorable binding properties with a K_{D} of 27 nM to HLA-A*01:01/KK-LC-1 and was further tested for binding to the other physiologically relevant off-target peptide complexes, i.e., HLA-A*01:01 with ICE1 (SEQ ID NO: 55), PTS (SEQ ID NO: 56), FUS (SEQ ID NO: 59), PTPRC (SEQ ID NO: 60), ROBO1 (SEQ ID NO: 61), TBL3 (SEQ ID NO: 62), CHTOP (SEQ ID NO: 63), DMXL2 (SEQ ID NO: 64), FAP (SEQ ID NO: 65), GPAM (SEQ ID NO: 66), PDE10A (SEQ ID NO: 67), VAV1 (SEQ ID NO: 68), ZBTB40 (SEQ ID NO: 69) and ZNF430 (SEQ ID NO: 70). No binding was observed for all tested off-target complexes, except HLA-A*01:01/GPAM (SEQ ID NO: 66), for which a low affinity interaction of K_{D} 171 nM was detected (Table 6, see also Scheifele, F. et al, Novel antibodies against a KK-LC-1-derived peptide presented on HLA-A*01 on tumor cells [abstract]. In: Proceedings of the American Association for Cancer Research Annual Meeting 2024; Part 2 (Late-Breaking, Clinical Trial, and Invited Abstracts); 2024 Apr. 5-10; San Diego, CA. Philadelphia (PA): AACR; Cancer Res 2024; 84 (7_Suppl): Abstract nr LB442 and FIG. 7 in according poster)).

TABLE 6
K_{D}-values of M2883 interaction with HLA-A*01:01 complexes loaded
with target KK-LC-1 peptide or off-target physiologically relevant
peptides posing a cross-reactivity risk.

| pMHC ligand | Affinity, K_{D} value (nM) |
|---|---|
| HLA-A*01:01/KK-LC-1 (SEQ ID NO: 54) | 27 |
| HLA-A*01:01/ICE1 (SEQ ID NO: 55) | n.b. |
| HLA-A*01:01/PTS (SEQ ID NO: 56) | n.b. |
| HLA-A*01:01/FUS (SEQ ID NO: 59) | n.b. |
| HLA-A*01:01/PTPRC (SEQ ID NO: 60) | n.b. |
| HLA-A*01:01/ROBO1 (SEQ ID NO: 61) | n.b. |
| HLA-A*01:01/TBL3 (SEQ ID NO: 62) | n.b. |
| HLA-A*01:01/CHTOP (SEQ ID NO: 63) | n.b. |
| HLA-A*01:01/DMXL2 (SEQ ID NO: 64) | n.b. |
| HLA-A*01:01/FAP (SEQ ID NO: 65) | n.b. |
| HLA-A*01:01/GPAM (SEQ ID NO: 66) | 171 |
| HLA-A*01:01/PDE10A (SEQ ID NO: 67) | n.b. |
| HLA-A*01:01/VAV1 (SEQ ID NO: 68) | n.b. |
| HLA-A*01:01/ZBTB40 (SEQ ID NO: 69) | n.b. |
| HLA-A*01:01/ZNF430 (SEQ ID NO: 70) | n.b. |

n.b.-no binding detected

The binding recognition motif of M2759, M2769 and M2883 was determined by alanine scan mutagenesis of the target peptide and SPR measurements. Additionally, M2883 was further analyzed in arginine and aspartic acid scan mutagenesis. Briefly, each residue of the KK-LC-1 peptide NTDNNLAVY (SEQ ID NO: 54) was replaced with alanine (with exception of position 7, which was replaced by serine), arginine or aspartic acid (with exception of position 3, which was replaced by serine instead of aspartic acid). The kinetic parameters for each peptide-MHC were determined by SPR, according to the previously described method. K_{D}-values for each tested peptide loaded on HLA-A*01:01 are shown in Table 7. M2883 displayed a very broad binding profile with at least 6 peptide residues showing relevance for binding. Affinity of M2883 to the pMHC complex was drastically reduced (K_{D}-values increased) when amino acids on positions 5, 6, and 8 of the peptide were mutated to alanine and position 7 to serine; positions 5, 6, 7, 8, and 9 were mutated to arginine, and positions 4, 5, 6, and 7 were mutated to aspartate. Additionally, moderate affinity reduction with 2-4-fold K_{D}-value increase was observed when position 3 was mutated to alanine or serine, position 4 to alanine, position 8 to aspartate, and position 9 to alanine or aspartate. Affinity increase of about 5-fold was observed when position 4 was mutated to arginine. M2769 and M2759 showed moderate to drastic affinity reduction when positions 3, 4, 5, 6, 8 and 9 were mutated to alanine and position 7 to serine, showing similar behavior to M2883 (see also Scheifele, F. et al, Novel antibodies against a KK-LC-1-derived peptide presented on HLA-A*01 on tumor cells [abstract]. In: Proceedings of the American Association for Cancer Research Annual Meeting 2024; Part 2 (Late-Breaking, Clinical Trial, and Invited Abstracts); 2024 Apr. 5-10; San Diego, CA. Philadelphia (PA): AACR; Cancer Res 2024; 84 (7_Suppl): Abstract nr LB442 and according poster).

TABLE 7
Affinity of M2883, M2769 and M2759 to HLA-A*01:01 complexes comprising
KK-LC-1 alanine and/or arginine and aspartic acid mutagenesis.

| | | | Affinity to peptide-loaded HLA- | | |
|---|---|---|---|---|---|
| | Peptide | | A*01:01, K_{D} (nM) | | |
| Ligand | sequence | SEQ ID NO | M2883 | M2769 | M2759 |
| KK-LC- | NTDNNLAVY | SEQ ID NO: 54 | 27 | 191 | 84 |
| 1 | | | | | |
| N1A | ATDNNLAVY | SEQ ID NO: 94 | 28 | 212 | 92 |
| T2A | NADNNLAVY | SEQ ID NO: 95 | 24 | 178 | 82 |
| D3A | NTANNLAVY | SEQ ID NO: 96 | 74 | 341 | 126 |
| N4A | NTDANLAVY | SEQ ID NO: 97 | 94 | 547 | 263 |
| N5A | NTDNALAVY | SEQ ID NO: 98 | 564 | n.b. | 1751 |
| L6A | NTDNNAAVY | SEQ ID NO: 99 | n.b. | n.b. | n.b. |
| A7S | NTDNNLSVY | SEQ ID NO: 100 | 291 | 1883 | 876 |
| V8A | NTDNNLAAY | SEQ ID NO: 101 | 472 | n.b. | 1826 |
| Y9A | NTDNNLAVA | SEQ ID NO: 102 | 108 | 634 | 284 |
| N1R | RTDNNLAVY | SEQ ID NO: 103 | 23 | n.d. | n.d. |
| T2R | NRDNNLAVY | SEQ ID NO: 104 | 31 | n.d. | n.d. |
| D3R | NTRNNLAVY | SEQ ID NO: 105 | 21 | n.d. | n.d. |
| N4R | NTDRNLAVY | SEQ ID NO: 106 | 5.4 | n.d. | n.d. |
| N5R | NTDNRLAVY | SEQ ID NO: 107 | n.b. | n.d. | n.d. |
| L6R | NTDNNRAVY | SEQ ID NO: 108 | 2003 | n.d. | n.d. |
| A7R | NTDNNLRVY | SEQ ID NO: 109 | n.b. | n.d. | n.d. |
| V8R | NTDNNLARY | SEQ ID NO: 110 | n.b. | n.d. | n.d. |
| Y9R | NTDNNLAVR | SEQ ID NO: 111 | n.b. | n.d. | n.d. |
| N1D | DTDNNLAVY | SEQ ID NO: 112 | 30 | n.d. | n.d. |
| T2D | NDDNNLAVY | SEQ ID NO: 113 | 24 | n.d. | n.d. |
| D3S | NTSNNLAVY | SEQ ID NO: 114 | 167 | n.d. | n.d. |
| N4D | NTDDNLAVY | SEQ ID NO: 115 | 1751 | n.d. | n.d. |
| N5D | NTDNDLAVY | SEQ ID NO: 116 | n.b. | n.d. | n.d. |
| L6D | NTDNNDAVY | SEQ ID NO: 117 | n.b. | n.d. | n.d. |
| A7D | NTDNNLDVY | SEQ ID NO: 118 | n.b. | n.d. | n.d. |
| V8D | NTDNNLADY | SEQ ID NO: 119 | 75 | n.d. | n.d. |
| Y9D | NTDNNLAVD | SEQ ID NO: 120 | 101 | n.d. | n.d. |

n.b.-no binding detected; n.d.-not determined

### Example 9—KK-LC-1-Specific T Cell Activation on Peptide-Pulsed T2A1 Cells by HLA-A*01:01/KK-LC-1×CD3 Bispecific Antibodies

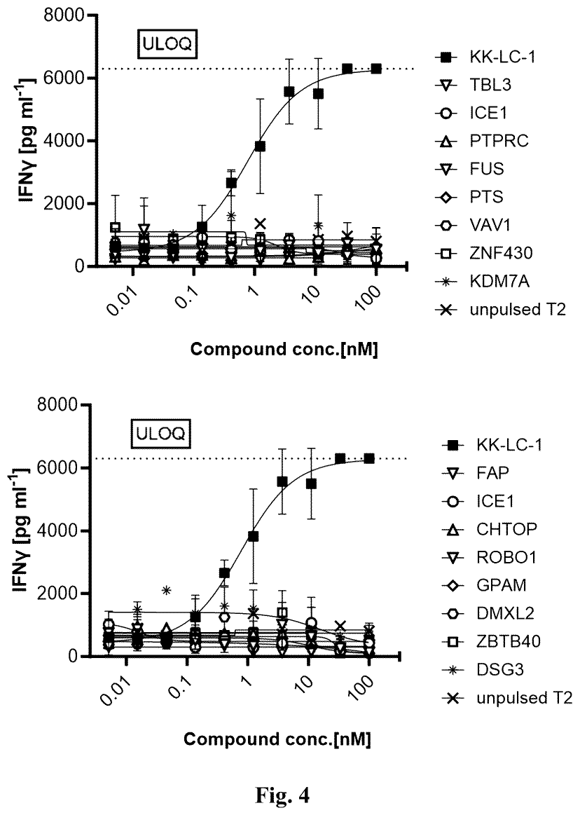

Tap-deficient T2 cells were transduced with a lentiviral vector encoding HLA-A*01:01 and beta-2 microglobulin and carrying the puromycin resistance gene (VectorBuilder). T2 cells were seeded in 6-well plates at 200,000 cells per well and transduced with lentiviral particles at MOI 10. Selection of HLA-A*01:01-expressing cells (T2A1) in puromycin-containing medium was initiated after 48 h and continued for at least 2 weeks before cells were used in experiments. T2A1 cells were incubated with serum-free RPMI1640 medium containing peptides KK-LC-1 (SEQ ID NO: 54), TBL3 (SEQ ID NO: 62), ICE1 (SEQ ID NO: 55), PTPRC (SEQ ID NO: 60), FUS (SEQ ID NO: 59), PTS (SEQ ID NO: 56), VAV1 (SEQ ID NO: 68), ZNF430 (SEQ ID NO: 70), KDM7A (SEQ ID NO: 58), FAP (SEQ ID NO: 65), PDE10A (SEQ ID NO: 67), CHTOP (SEQ ID NO: 63), ROBO1 (SEQ ID NO: 61), GPAM (SEQ ID NO: 66), DMXL2 (SEQ ID NO: 64), ZBTB40 (SEQ ID NO: 69) or DSG3 (SEQ ID NO: 57) at a concentration of 10 μM overnight in 96 wells plates. Then, cells were washed in complete RPMI 1640 medium and co-incubated with PBMCs (E:T ratio of 5:1) and varying concentrations of M2883 (range of 0.005 nM-100 nM) for 24 h. T cell activation was determined by quantification of IFN-gamma in the cell supernatants and is shown in FIG. 4. Specific T cell activation was observed for M2883 in the presence of the KK-LC-1 peptide, while no activation was detected in the presence of any of the tested off-target peptides, indicating a very specific profile of M2883.

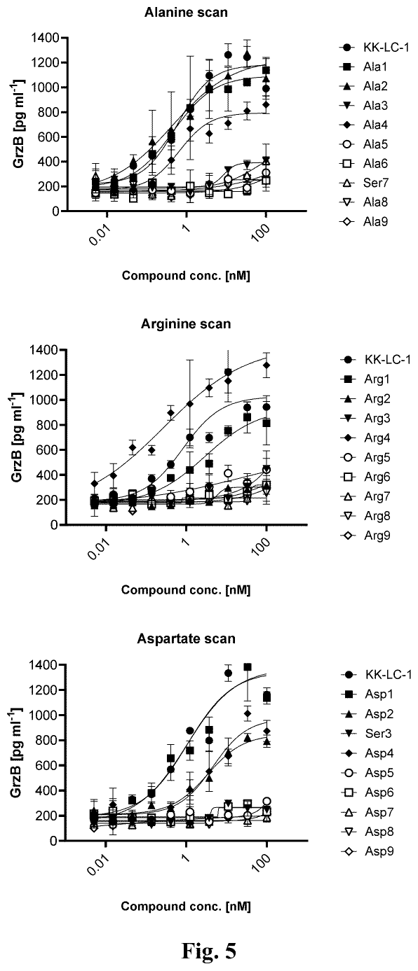

To confirm the binding recognition motif of M2883 previously determined by affinity measurements (Example 8), alanine, arginine and aspartic acid scan mutagenesis of the target KK-LC-1 peptide was also performed in antigen presenting T2A1 cells. Briefly, T2A1 cells were incubated with complete RPMI1640 medium containing KK-LC-1 target peptide and the respective alanine, arginine and aspartic acid mutated peptides (Table 7) at a concentration of 10 μM in 384 wells plates. Then, cells were co-incubated with PBMCs (E:T ratio of 1:1) and M2883 at varying concentrations (range of 0.005 nM-100 nM) for 24 h. T cell activation was determined by quantification of Granzyme B from cell supernatants, using the human Granzyme B ELISA kit (MabTech) according to the manufacturer's instructions. Corresponding results are shown in FIG. 5. M2883 showed an excellent specificity profile with at least 6 amino acid positions of the native KK-LC-1 peptide relevant for T cell activation. This outcome was consistent with the M2883 binding recognition motif previously determined by affinity measurements (SPR, Example 8).

### Example 10—In Vitro Efficacy of HLA-A*01:01/KK-LC-1×CD3 Bispecific Antibodies On Cancer Cell Lines

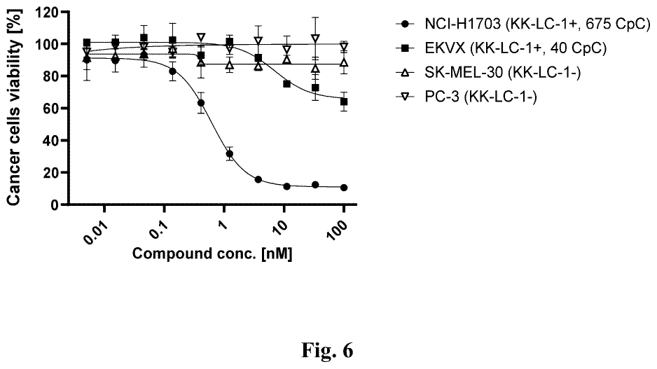

Cancer cell killing mediated by M2883 was analyzed in a time-resolved manner using the IncuCyte S3 system. Briefly, HLA-A*01:01-positive KK-LC-1-positive (NCI-H1703 and EKVX) and HLA-A*01:01-positive KK-LC-1-negative (SK-MEL-30 and PC-3) cancer cells were transduced with Nuclight Red lentivirus (Sartorius) to stably express the mKate2 fluorescent protein. Cancer cells were seeded at the density of 1.5×10³ cells per well in a sterile 384-well flat bottom adhesion tissue culture plate overnight at 37° C. and 5% CO₂ in an incubator. Molecule M2883 was added at the indicated concentrations (range of 0.005 nM-100 nM). PBMCs were added as effector cells to each well at an E:T ratio of 10:1. The plate was imaged by fluorescent microscopy to monitor cell growth for 72 h. The degree of cell killing was quantified by comparing the fold growth ratio of fluorescent target cancer cells over time, relative to their number at time 0. As depicted in FIG. 6, molecule M2883 showed a very specific cancer cell killing of the KK-LC-1-positive cancer cells and no killing of the KK-LC-1-negative cancer cells. Good correlation of potency and target copy number was observed.

### Example 11—In Vitro Safety of HLA-A*01:01/KK-LC-1×CD3 Bispecific Antibodies

Safety of M2883 was tested on various primary cell types expressing HLA-A*01:01. Tested cells included Human Cardiac Microvascular Endothelial Cells (HCMEC_147), Human Bronchial Smooth Muscle Cells (HBSMC_297), Human Small Airway Epithelial Cells (HSAEpC_645) and Human Pulmonary Microvascular Endothelial Cells (HPMEC_770). Cells were prepared in assay medium (RPMI 1640 containing 10% FBS and 1% penicillin-streptomycin) and plated at 20,000 cells per well in a volume of 50 μL assay medium. PBMCs effector cells were plated at 100,000 cells per well in a volume of 50 μL assay medium. Varying concentrations of compound M2883 (range of 0.05 nM to 100 nM) were added to the plated wells in 15 μL assay volume. The final assay medium was made up to 150 μL per well. All reactions were performed in duplicates. The plates were incubated for 24 h at 37° C./5% CO2. Supernatants were collected and analyzed by human Granzyme B ELISA kit (MabTech) according to the manufacturer's instructions. HLA-A*01:01-positive and KK-LC-1-positive cell line NCI-H1703 served as a positive control. Corresponding results are depicted in FIG. 7A. M2883 showed no reactivity and therefore a safe profile on all four tested primary human cells.

M2883 was further tested on an extended selection of human primary cells, according to the above-described protocol. Tested cells included human cardiac fibroblasts (HCF_722), normal human lung fibroblasts (NHLF_19232) and human aortic smooth muscle cells (HAoSMC_173). Human pulmonary fibroblasts (HPF_646) served as an HLA-A*01:01-negative, KK-LC-1-negative control and cancer cell line NCI-H1703 as an HLA-A*01:01-positive, KK-LC-1-positive control. Corresponding results are depicted in FIG. 7B. Granzyme B release was observed in fibroblast cells HCF_722 and NHLF 19232 when treated with high concentrations of M2883. Both reacting primary cells showed lower reactivity compared to the positive control NCI-H1703 with about 10-fold difference in EC50 value. HAoSMC_173 showed only minor and negligible reactivity at the highest tested concentration of M2883. HLA-A*01:01-negative fibroblast cells HPF_646 showed no reactivity when treated with M2883, suggesting that the observed reactivity in NHLF_19232 and HCF_722 could be caused by HLA-A*01:01 specific off-target peptide. Further engineering of M2883 was performed to address the off-target reactivity observed in HLA-A*01:01-positive primary fibroblast cells.

### Example 12—Optimization of HLA-A*01:01/KK-LC-1×CD3 Bispecific Antibody M2883

Variants of M2883 were generated and tested for binding to HLA-A*01:01 loaded with KK-LC-1, KK-LC-1_N4R and GPAM derived peptides. Binding affinity was determined by SPR, as described previously. M2883 variant with VL_E27K substitution was identified, exhibiting a substantial reduction in binding affinity to KK-LC-1_N4R (K_{D} of 201 nM) and GPAM (no detectable binding), while retaining the parental-like binding affinity to the target KK-LC-1 peptide (K_{D} of 36 nM). Further optimization of the M2883 VL_E27K variant was performed to enhance humanization, stability and producibility. This included affinity maturation and optimization of framework regions. Further germlining of the M2883 VL_E27K variant led to the development of M3591, which exhibited a significantly higher human germline sequence identity score of 87.9%, compared to 80.2% of the parental molecule M2883. This identity score was determined based on the percentage of amino acid sequence identity between the aligned regions and their corresponding germline sequences. In parallel to framework optimization, the affinity maturation of M2883 VL_E27K molecule was performed. Briefly, several phage display scFv libraries were constructed with targeted randomizations in CDRs. These libraries were designed to introduce targeted diversity while preserving the structural integrity of the scFv framework, facilitating the identification of high-affinity antigen binders. The scFv libraries were synthesized and cloned into a phagemid vector upstream of a gene encoding the minor coat protein pIII. The phagemid libraries were then transformed into E. coli TG1 cells and the number of colonies growing on the plates was used to determine the library diversity, assuring proper representation of variant sequences. The resulting libraries were subjected to three rounds of biopanning with counterselection against a mix of HLA-A*01:01/KK-LC-1_N4R, GPAM and DCAF13 (SEQ ID NOs: 106, 66 and 126, respectively), and selection against HLA-A*01:01/KK-LC-1 antigen. Libraries were screened for hits using a monoclonal phage ELISA after second and third round of panning. Hits showing high binding to HLA-A*01:01/KK-LC-1, low binding to HLA-A*01:01/KK-LC-1_N4R, and no binding to HLA-A*01:01/GPAM and HLA-A*01:01/DCAF13 were sequenced. Sequence analysis facilitated the identification of unique clones which were then expressed and further characterized in a Fab-scFv bispecific format of anti-CD3 Fab×anti-KK-LC-1 scFv. Substitutions identified to improve affinity, while retaining the desired specificity profile were incorporated into M3591, leading to the development of M3903.

### Example 13—Characterization of the Optimized HLA-A*01:01/KK-LC-1×CD3 Bispecific Antibody M3903

Affinity matured and optimized M3903, the humanization intermediate M3591 and the parental M2883 were subjected to a characterization of binding affinity and biophysical properties. A summary of key improvements in biophysical properties, binding affinity and specificity is presented in Table 8. Affinity to the target antigen HLA-A*01:01/KK-LC-1 and the relevant off-target similar peptide antigen HLA-A*01:01/GPAM was evaluated using previously described methods. With a K_{D} of 1.8 nM, M3903 showed a 15-fold increase in binding affinity to KK-LC-1, compared to the parental binder M2883. M3903 also demonstrated improved specificity to the off-target similar peptide complex HLA-A*01:01/GPAM, which contains a positively charged residue at position 4, a feature considered important for further enhancing specificity. Specificity window between HLA-A*01:01/KK-LC-1 and HLA-A*01:01/GPAM binding was significantly improved through engineering from approximately 6-fold in M2883 and about 40-fold in M3591 to over 260-fold in M3903.

Thermostability of M2883, M3591 and M3903 was measured using differential scanning fluorimetry (DSF). For this purpose, the samples were diluted to a concentration of 0.5 mg/ml. Temperature increased by 1° C./min from 20-95° C. Data were analyzed with the PR Panta Analysis (×64) software. Thermostability including onset of protein unfolding (Tₒₙₛₑₜ), melting temperature Tm and aggregation temperature (T_{agg}) was determined. In molecules consisting of several domains, such as bispecific compounds in Fab-scFv format, melting temperature corresponding to the individual domains can be determined. The Fab domain typically shows Tₘ of >75° C. Here, the Tₘ of scFv relates to the first inflection point in the melting curve of the tested molecule. The results are shown in Table 8. M3903 showed superior thermostability, compared to both precursors and a very high melting temperature of 75.2° C. with only one inflection point. Molecule cumulant radius (average size of the particles) of M2883, M3591 and M3903 was determined by dynamic light scattering (DLS) using the Prometheus Panta instrument.

Briefly, samples were mixed and 0.1 μm filtered before measurement. Analysis was performed at 20° C. and at the DLS laser power determined by the discovery scan of the instrument. Data was analyzed with the PR Panta Analysis (×64) software. The results are shown in Table 8. The DLS measurements revealed that M2883 had the largest cumulant radius (4.0 nm), followed by M3591 (3.9 nm) and M3903 (3.7 nm), indicating a slight but consistent decrease in hydrodynamic size following molecule engineering. This trend suggests that M3903 could have a more compact structure and/or reduced self-association compared to the parental variants-both desirable qualities in therapeutic biologics.

TABLE 8
Key feature comparison of M2883, M3591 and M3903.

| Feature | M2883 | M3591 | M3903 |
|---|---|---|---|
| Human germline identity (scFv, %) | 80.2 | 87.9 | 87.4 |
| Affinity to HLA-A*01:01/ | 27 | 15 | 1.8 |
| KK-LC-1 (K_{D}, nM) | | | |
| Affinity to HLA-A*01:01/ | 171 | 594 | 472 |
| GPAM (K_{D}, nM) | | | |
| Tₒₙₛₑₜ [° C.] | 54.4 | 59.9 | 63.3 |
| Tₘ scFv [° C.] | 63.1 | 65.8 | 75.2 |
| T_{agg} [° C.] | 73.8 | 74.2 | 73.7 |
| Cumulant radius [nm] | 4.0 | 3.9 | 3.7 |

M3903 was additionally tested for binding to the HLA-A*01:01 complexes loaded with off-target peptides of physiological relevance and high sequence identity to KK-LC-1 peptide. Tested ligands included HLA-A*01:01/DSG3, HLA-A*01:01/PTS, HLA-A*01:01/KDM7A, HLA-A*01:01/ICE1, HLA-A*01:01/ROBO1, HLA-A*01:01/TBL3 and a positive control HLA-A*01:01/KK-LC-1. Binding affinity was determined by SPR using a previously described protocol. The resulting K_{D} values are shown in Table 9. No binding or only low affinity interaction with at least 17-fold difference in binding affinity compared to the target HLA-A*01:01/KK-LC-1 was detected for the tested off-target peptide complexes.

TABLE 9
K_{D}-values of M3903 interaction with HLA-A*01:01
complexes loaded with target KK-LC-1 peptide
or off-target physiologically relevant peptides.

| | Affinity, K_{D} |
|---|---|
| pMHC ligand | value (nM) |
| HLA-A*01:01/KK-LC-1 (SEQ ID NO: 54) | 1.8 |
| HLA-A*01:01/DSG3 (SEQ ID NO: 57) | n.b. |
| HLA-A*01:01/PTS (SEQ ID NO: 56) | n.b. |
| HLA-A*01:01/KDM7A (SEQ ID NO: 58) | 32 |
| HLA-A*01:01/ICE1 (SEQ ID NO: 55) | n.b. |
| HLA-A*01:01/ROBO1 (SEQ ID NO: 61) | n.b. |
| HLA-A*01:01/TBL3 (SEQ ID NO: 62) | 511 |

n.b.-no binding detected

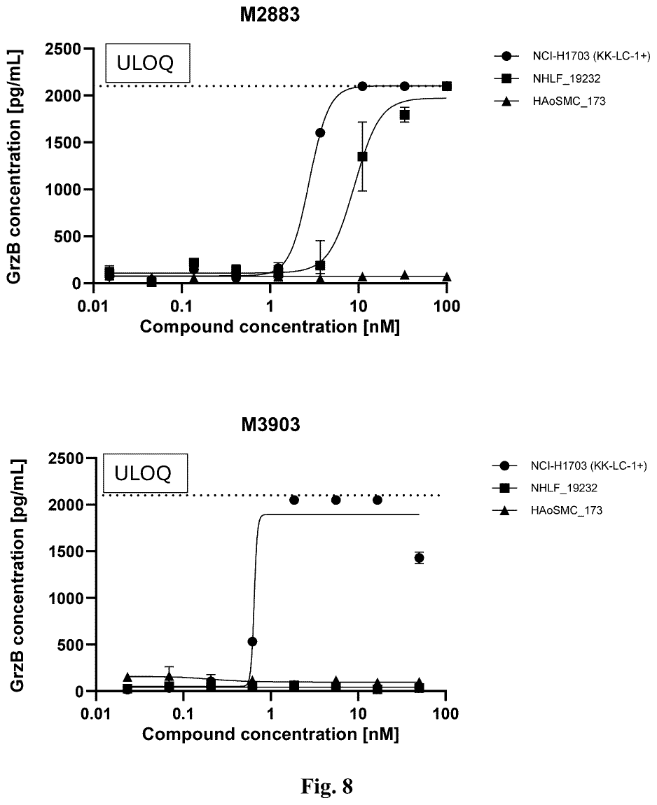

Safety of M3903 was evaluated in a primary cell assay, essentially as described in Example 11, and compared to the parental molecule M2883. Tested cells included human lung fibroblasts (NHLF_19232), human aortic smooth muscle cells (HAoSMC_173) and the HLA-A*01:01-positive, KK-LC-1-positive control cancer cell line NCI-H1703. Corresponding results are shown in FIG. 8. M3903 showed higher potency on the antigen-positive NCI-H1703 cells than the parental M2883 and no reactivity on the tested primary cells. This demonstrates not only improved efficacy but also greater specificity and a favorable safety profile of M3903.

### Example 14—Characterization of the Reformatted HLA-A*01:01/KK-LC-1×CD3 Bivalent Bispecific T Cell Engager M3904

M3904 was created by reformatting of M3903 into a bivalent bispecific T cell engager in a Fab-(scFv)₂ format with anti-CD3 Fab and two anti-HLA-A*01:01/KK-LC-1 scFvs. This format enables efficient avidity-driven targeting of antigen-expressing cancer cells. Furthermore, with a size of 100 kDa, it is expected to exhibit a favorable pharmacokinetic profile, potentially supporting Q2W or Q3W dosing.

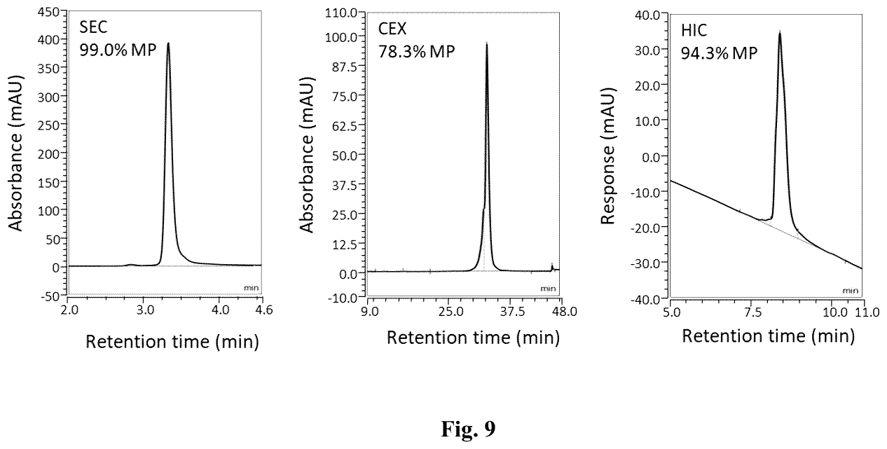

Bivalent bispecific M3904 was produced using stable CHO pools in 14-day fed-batch cultivations. Target protein was purified from clarified, sterile-filtered culture supernatants via affinity chromatography and cation exchange chromatography. Final protein purity was evaluated using SE-HPLC, CEX HPLC, and HIC HPLC (FIG. 9). M3904 showed a good manufacturability profile with a titer of over 3 g/L and a high product quality.

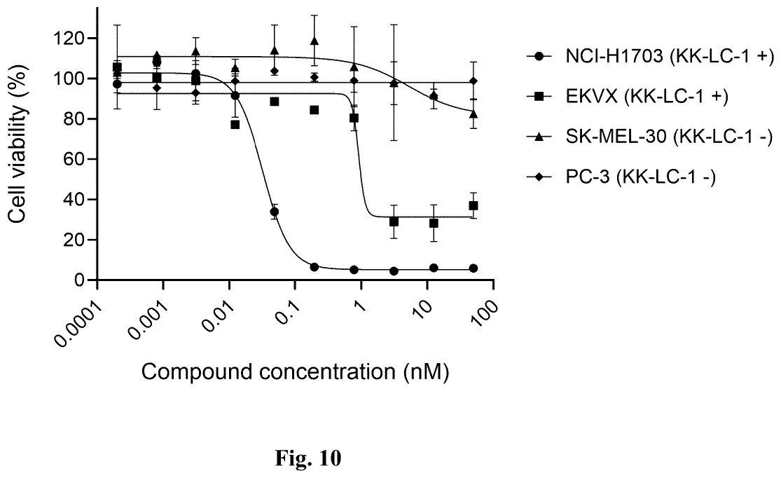

Potency of M3904 was measured in a cell killing experiment, essentially as described in Example 10. Tested cancer cells included NCI-H1703 and EKVX, which are both HLA-A*01:01-positive and KK-LC-1-positive, SK-MEL-30 and PC-3, which are both HLA-A*01:01-positive and KK-LC-1-negative and served as negative controls. Results are shown in FIG. 10. M3904 showed a potent and specific cancer cell killing of the KK-LC-1-positive cancer cells and no killing of the KK-LC-1-negative cancer cells. Compared to M2883 (FIG. 6), M3904 displayed a more efficient killing of both tested antigen-positive cell lines and a good correlation between potency and target copy number.

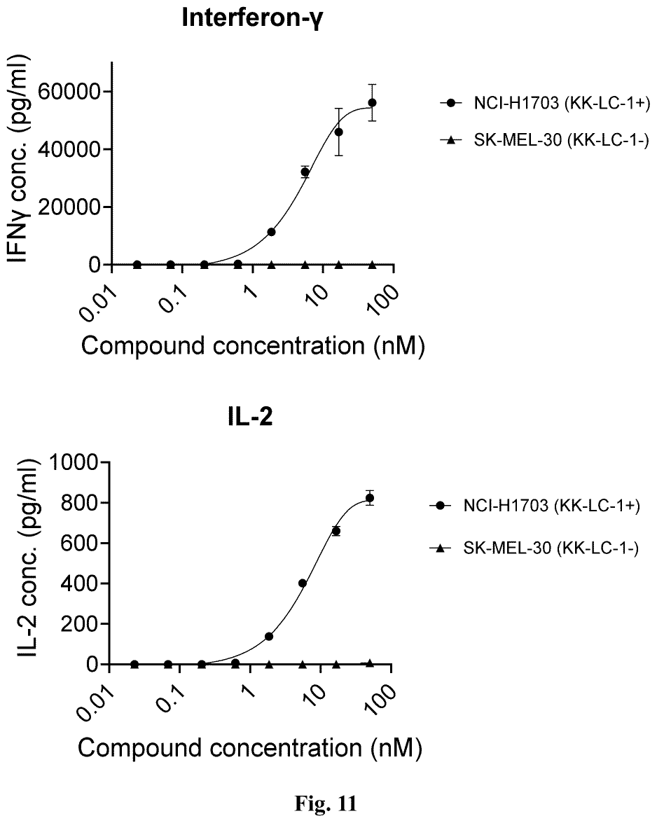

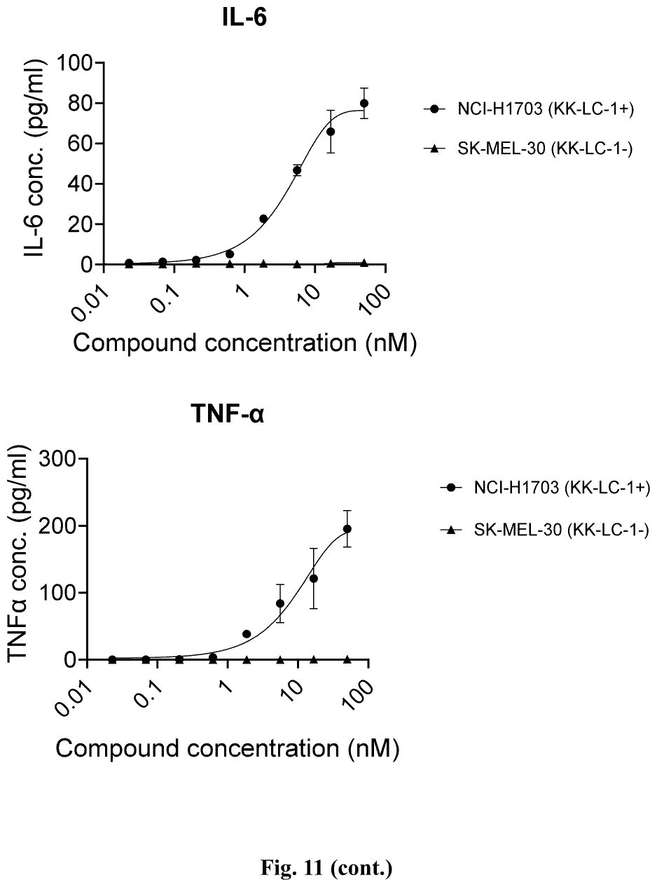

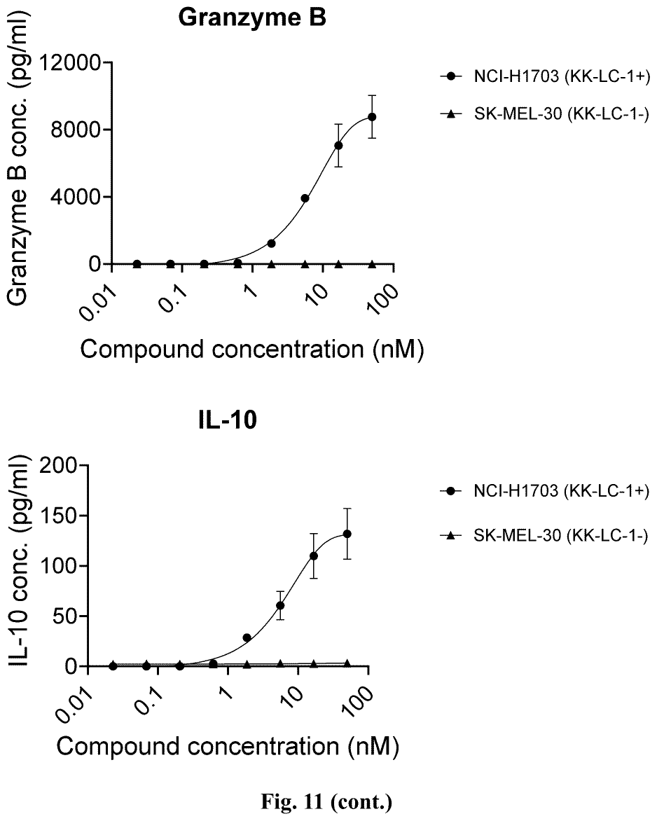

M3904-mediated T cell activation, assessed by cytokine and cytolytic molecule release was determined in a coculture cellular assay. Briefly, HLA-A*01:01-positive and KK-LC-1-positive cell line NCI-H1703 and HLA-A*01:01-positive and KK-LC-1-negative cell line SK-MEL-30 were prepared in assay medium (RPMI 1640 containing 10% FBS and 1% penicillin-streptomycin) and plated at 20,000 cells per well in a volume of 100 μL assay medium. The following day, the medium was aspirated and PBMCs effector cells were plated at 100,000 cells per well in a volume of 90 μL assay medium. Varying concentrations of compound M3904 (range of 0.022 nM to 50 nM) were added to the plated wells in 10 μL assay volume. The final assay medium was topped up to 100 μL per well. All reactions were performed in duplicates. The plates were incubated for 24 h at 37° C./5% CO2. Supernatants were collected and IFNγ, IL-2, IL-6, TNFα, Granzyme B and IL-10 were analyzed with MSD U-plex plates (Meso Scale Discovery) according to the manufacturer's instructions. IFNγ, IL-2, IL-6, TNFα, Granzyme B, and IL-10 were detected only in NCI-H1703 co-culture supernatants following M3904 treatment, as shown in FIG. 11, indicating a specific and robust activation, and functional response of T cells in vitro.

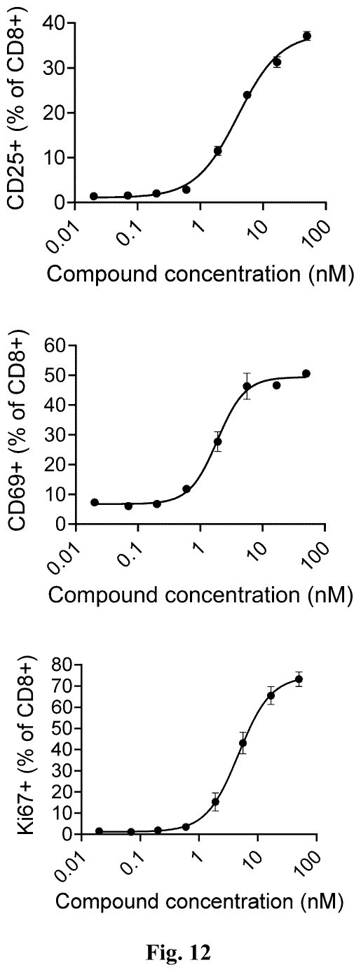

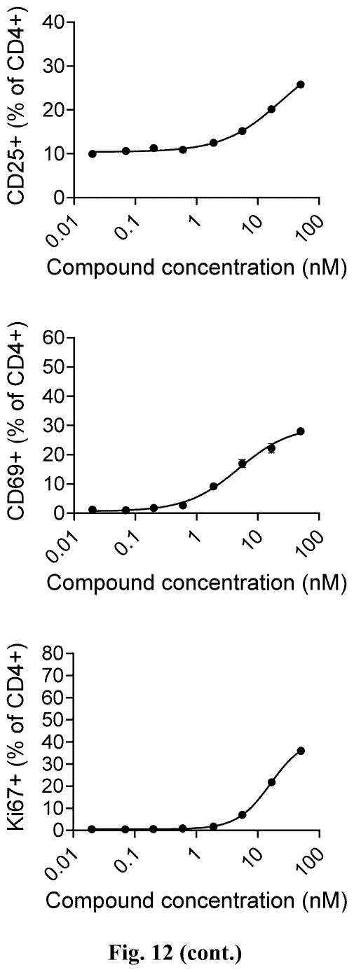

M3904-mediated T cell activation markers were analyzed in a coculture cellular assay. Briefly, HLA-A*01:01-positive and KK-LC-1-positive cells NCI-H1703 were prepared in assay medium (RPMI 1640 containing 10% FBS and 1% penicillin-streptomycin) and plated at 20,000 cells per well in a volume of 50 μL assay medium. PBMCs effector cells were plated at 100,000 cells per well in a volume of 50 μL assay medium. Varying concentrations of compound M3904 (range of 0.02 nM to 50 nM) were added to the plated wells in 15 μL assay volume. The final assay medium was topped up to 150 μL per well. All reactions were performed in duplicates. Cells were incubated at 37° C./5% CO₂. Analysis was performed at 48 h and 96 h time-points for different activation markers. After 48 h, cells were collected and stained with Live/dead Fixable NIR and with the antibodies against CD3, CD4, CD8, together with CD69 and CD25 as activation markers. Samples were then fixed and stored at 4° C. until analyzed by flow cytometry using the MACSQuant Analyzer 10. Analysis of T cell populations and activation markers was performed using the FlowLogic software. After 96 h, cells were collected, permeabilized and stained with Live/dead Fixable NIR, together with antibodies against CD3, CD4, CD8 and Ki67 proliferation marker. Samples were then fixed and stored at 4° C. until analyzed by flow cytometry, as described above. Results are shown in FIG. 12. M3904 treatment induced upregulation of activation markers (CD69, CD25) and the proliferation marker Ki67 on CD4 and CD8+ cells, demonstrating potent in vitro T cell activation.

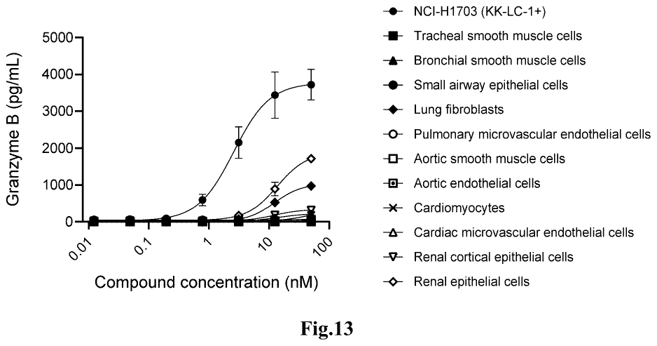

M3904 was tested for safety in a primary cell assay against an extended panel of HLA-A*01:01-positive human primary cells originating from critical tissues, namely lung, vascular system, heart and kidneys. Assays were performed essentially as described in Example 11. Tested cells included tracheal smooth muscle cells (HTSMC_36364), bronchial smooth muscle cells (HBSMC_750), small airway epithelial cells (HSAEpC_196), lung fibroblasts (NHLF_19232), pulmonary microvascular endothelial cells (HPMEC_770), aortic smooth muscle cells (HAoSMC_936), aortic endothelial cells (HAEC_22171), cardiomyocytes (HCM_844), cardiac microvascular endothelial cells (HCMEC_147), renal cortical epithelial cells (HRCEpC_897) and renal epithelial cells (HREpC_947). HLA-A*01:01-positive and KK-LC-1-positive cell line NCI-H1703 served as a positive control. Corresponding results are depicted in FIG. 13. Low levels of Granzyme B were detected at the highest tested M3904 concentrations in lung fibroblasts and in renal epithelial cells, while all other tested primary cells showed no or negligible Granzyme B release at any tested M3904 concentration. These results support a low risk for M3904 off-target reactivity and a favorable safety profile.

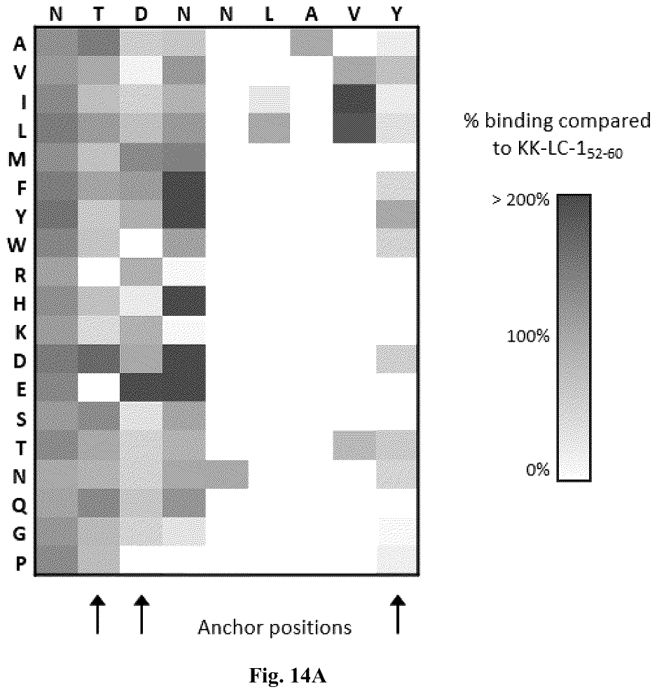

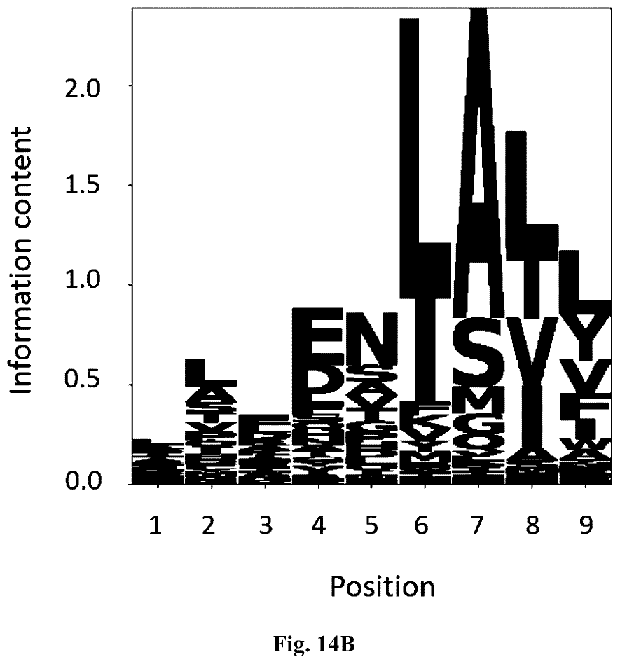

To further characterize the off-target reactivity risk of M3904, binding profile analysis using an X-scanning mutagenesis (X-scan) of the target KK-LC-1 peptide was performed. In this approach, each position in the peptide was systematically substituted by all naturally occurring amino acids, excluding cysteine. The binding of M3903, the monovalent counterpart of M3904, to the mutated KK-LC-1 peptide variants was assessed by SPR, using the previously described method. A heat map obtained from the X-scan analysis representing the change in binding affinity relative to the native KK-LC-1 peptide was generated and is shown in FIG. 14A. The corresponding sequence logo depicting the peptide recognition profile of M3903 is shown in FIG. 14B. Generated data reveal distinct binding constraints of M3903 across the peptide positions. Binding specificity of M3903 is strongly dictated by positions 5, 6, 7, 8, and 9, suggesting that these residues play a critical role in M3903 binding. In particular, positions 5, 6, 7 and 8 show a strong preference for the native KK-LC-1 amino acid, while position 9 allows for substitutions with amino acids of similar size or chemical properties. Conversely, positions 1, 2, 3, and 4 allow greater variability and can accommodate multiple amino acids with no drop in binding affinity. Notably, certain substitutions at position 4 can enhance binding affinity. These findings provide key insights into the specificity and potential off-target interactions of M3903.

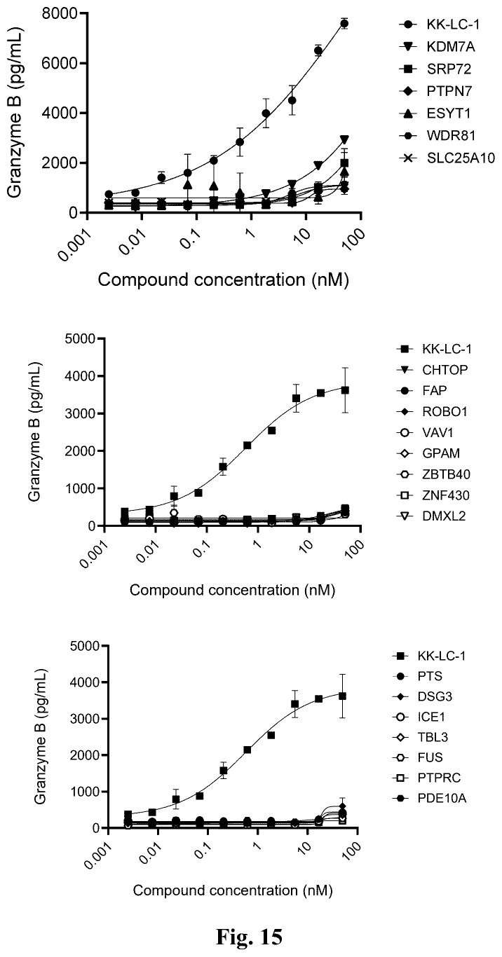

Based on the binding profile of M3903, additional off-target peptides were tested, including KDM7A (SEQ ID NO: 58), SRP72 (SEQ ID NO: 71), PTPN7 (SEQ ID NO: 72), ESYT1 (SEQ ID NO: 73), WDR81 (SEQ ID NO: 74), and SLC25A10 (SEQ ID NO: 75). These and the previously identified endogenous physiologically relevant peptides with high sequence similarity to KK-LC-1 (Table 3) were tested in a cytolytic molecule release assay using the peptide-pulsed T2A1 cells, essentially as described in Example 9. KK-LC-1 peptide served as a positive control. Resulting data are presented in FIG. 15. M3904 exhibited a highly specific profile, characterized by high GrzB release in the presence of the KK-LC-1 peptide. Only at high concentrations M3904 triggered minor GrzB release with the off-target peptides KDM7A, SRP72, PTPN7, ESYT1, WDR81, and SLC25A10. Among these, KDM7A induced the highest GrzB release, though it still maintained a large safety margin compared to the target KK-LC-1 peptide. No Granzyme B release was detected in the presence of other tested off-target peptides, indicating a very specific profile of M3904.

The developability profile of M3904 was assessed. To evaluate M3904 solubility, the compound was concentrated to 140 mg/ml in a Histidine buffer and analyzed by size exclusion chromatography (SEC). Ultra-high concentrations were reached without any detectable oligomerization (FIG. 16A). Accelerated shelf-life stability of M3904 was assessed in the same buffer at 50 mg/ml and 37° C. for one week. The samples were analyzed by SEC and demonstrated good stability (FIG. 16B). Additionally, an incubation was performed in artificial subcutaneous fluid buffer (Schuster et al. 2021) at simulated physiological conditions (34° C., pH 7.4). The incubated samples were analyzed by SEC and virtually no degradation was detected even after two weeks (FIG. 16C). These data support the feasibility of a high drug product concentration which allows subcutaneous administration.

1. An antigen binding protein which specifically binds to a major histocompatibility complex (MHC)-displayed NTDNNLAVY (SEQ ID NO: 54), wherein the antigen binding protein comprises:
(a) an antibody heavy chain variable (VH) domain comprising an HCDR1 amino acid sequence of SSYYYMC (SEQ ID NO: 25), an HCDR2 amino acid sequence of CIYAGSSGSTYYASWAKG (SEQ ID NO: 26), and an HCDR3 amino acid sequence of GAGYGNGGHSL (SEQ ID NO: 27); and
(b) an antibody light chain variable (VL) domain comprising an LCDR1 amino acid sequence of RASKNIYNSLA (SEQ ID NO: 28), an LCDR2 amino acid sequence of GASNLES (SEQ ID NO: 29), and an LCDR3 amino acid sequence of QATYYGHDNIGGA (SEQ ID NO: 30.

2. The antigen binding protein of claim 1, comprising:
(a) an antibody heavy chain variable (VH) domain comprising or consisting of the amino acid sequence that is at least about 90%, 95%, 96%, 97%, 98%, 99% or 100% identical to SEQ ID NO: 31; and
(b) an antibody light chain variable (VL) domain comprising or consisting of the amino acid sequence that is at least about 90%, 95%, 96%, 97%, 98%, 99% or 100% identical to SEQ ID NO: 32.

3. An antigen binding protein which specifically binds to a major histocompatibility complex (MHC)-displayed NTDNNLAVY (SEQ ID NO: 54), wherein the antigen binding protein comprises:
(a) an antibody heavy chain variable (VH) domain comprising or consisting of the amino acid sequence that is at least about 90%, 95%, 96%, 97%, 98%, 99% or 100% identical to SEQ ID NO: 31; and
(b) an antibody light chain variable (VL) domain comprising or consisting of the amino acid sequence that is at least about 90%, 95%, 96%, 97%, 98%, 99% or 100% identical to SEQ ID NO: 32.

4. The antigen binding protein of claim 3, wherein
(a) the amino acid sequence that is at least about 90%, 95%, 96%, 97%, 98%, or 99% identical to SEQ ID NO: 31 comprises one or more substitutions selected from the group consisting of A23T, A24V, S30R, R71K, N73T, L78V and G100aD; and/or
(b) the amino acid sequence that is at least about 90%, 95%, 96%, 97%, 98%, or 99% identical to SEQ ID NO: 32 comprises one or more substitution selected from the group consisting of DIE, Q3V, T20I, R24Q, K27E, K42Q, A43R, S63K, D70E, Y71F, S77D, Q79E, P80A, E81D, S83F, S83A, A90S, 195cV, C100G, K103E, V104L, E105V, E105T, I106V, K107R and K107L.

5. The antigen binding protein of claim 1, wherein:
the MHC is of HLA supertype A*01, in particular HLA-A*01:01;
having an affinity (K_{D}) to an MHC-displayed NTDNNLAVY (SEQ ID NO: 54) of at least about 30 nM or stronger;
the antigen binding protein shows low to no binding to MHC-displayed off-target peptides, optionally wherein the off-target peptide is selected from the group consisting of YTDNWLAVY (SEQ ID NO: 57), ETDNNIVVY (SEQ ID NO: 56), NTDNLLTEY (SEQ ID NO: 55), NSDSNLTTY (SEQ ID NO: 61), TADHNLLLY SEQ ID NO: 62), RTEEALQLY (SEQ ID NO: 71), FLHHTLALY (SEQ ID NO: 72), VTQESKAVY (SEQ ID NO: 73), MSEEHTAVY (SEQ ID NO: 74) and RTDGILALY (SEQ ID NO: 75);
the antigen binding protein shows at least about a 10-fold reduction in affinity (e.g., a 50-fold, 75-fold, 80-fold, 90-fold, 100-fold, 120-fold, 150-fold, or 200-fold decrease in affinity) to MHC-displayed TADHNLLLY (SEQ ID NO: 62), relative to affinity to MHC-displayed NTDNNLAVY (SEQ ID NO: 54); and/or
the antigen binding protein has at least a 50-fold decrease in affinity (e.g., a 75-fold, 80-fold, 90-fold, 100-fold, 120-fold, 150-fold, or 200-fold decrease in affinity) for the MHC-displayed peptides selected from the group consisting of SEQ ID NO: 55, SEQ ID NO: 56, SEQ ID NO: 57, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74 and SEQ ID NO: 75, relative to the affinity to MHC-displayed NTDNNLAVY (SEQ ID NO: 54).

6-10. (canceled)

11. The antigen binding protein of claim 1, being or comprising an antibody, such as a full-length immunoglobulin or an antibody fragment, optionally wherein the antibody fragment is a Fab, a Fab′, a F(ab′)₂, a scFv, a Fv fragment or a scFab.

12-13. (canceled)

14. The antigen binding protein of claim 1, being or comprising a scFv with an amino acid sequence that is at least about 90%, 95%, 96%, 97%, 98%, 99% or 100% identical to the amino acid sequence of SEQ ID NO: 33.

15. The antigen binding protein of claim 1, being chemically or biologically modified, optionally being glycosylated, PEGylated, PASylated, XTENylated or HESylated; and/or being linked to or combined with a functional entity, such as a detectable label, a therapeutic agent or a PK modifying moiety.

16-17. (canceled)

18. A chimeric antigen receptor (CAR) comprising the antigen binding protein of claim 1.

19. An immune cell expressing the CAR of claim 18, in particular wherein the immune cell is a T cell.

20. An antibody drug conjugate (ADC) comprising the antigen binding protein of claim 1.

21. (canceled)

22. A multispecific antigen binding protein that specifically binds to a major histocompatibility complex (MHC)-displayed NTDNNLAVY (SEQ ID NO: 54) and an immune cell, comprising
(1) a first and optionally a second antigen binding domain that binds to the MHC-displayed NTDNNLAVY (SEQ ID NO: 54), comprising:
(a) an antibody heavy chain variable (VH) domain comprising an HCDR1 amino acid sequence of SSYYYMC (SEQ ID NO: 25), an HCDR2 amino acid sequence of CIYAGSSGSTYYASWAKG (SEQ ID NO: 26), and an HCDR3 amino acid sequence of GAGYGNGGHSL (SEQ ID NO: 27); and
(b) an antibody light chain variable (VL) domain comprising an LCDR1 amino acid sequence of RASKNIYNSLA (SEQ ID NO: 28), an LCDR2 amino acid sequence of GASNLES (SEQ ID NO: 29), and an LCDR3 amino acid sequence of QATYYGHDNIGGA (SEQ ID NO: 30); and
(2) an immune cell binding domain.

23. The multispecific antigen binding protein of claim 22, wherein:
the multispecific antigen binding protein is bispecific or trispecific;
any one or more of the first and the second antigen binding domain and the immune cell binding domain is or comprises a full-length immunoglobulin or an antibody fragment, optionally wherein the antibody fragment is or comprises a Fab fragment, a F(ab′)₂ fragment, a Fab′ fragment, an Fv fragment, a single chain variable fragment (scFv), and a single domain antibody fragment;
the multispecific antigen binding protein is or comprises a (scFv)₂, BiTE, BIKE, Dart, diabody, Fab₂, or a Fab-scFv;
said second antigen binding domain is identical to the first antigen binding domain;
the multispecific antigen binding protein is or comprises a (scFv)₃, tribody, Fab₃, Fab₄, or scFv-Fab-scFv (Fab-scFv₂); and/or
the immune cell binding domain targets CD3, TCRα, TCRβ, the α/β T Cell Receptor, CD16a, NKG2D, CD94/NKG2C, NKD30, NKD46, CD89, CD64, and CD32a on the surface of an immune cell, in particular a human immune cell, optionally wherein the immune cell binding domain is an antibody, more particularly a CD3 binding domain, a CD16a binding domain, or BMA031 or a variant thereof.

24-31. (canceled)

32. The multispecific antigen binding protein of claim 23, wherein the CD3 binding domain comprises:
(a) an antibody heavy chain variable (VH) domain comprising an HCDR1 amino acid sequence of STYAMN (SEQ ID NO: 38) an HCDR2 amino acid sequence of RIRSKYNNYATYYADSVKG (SEQ ID NO: 39), and an HCDR3 amino acid sequence of HGNFGDSYVSWFAY (SEQ ID NO: 40); and
(b) an antibody light chain variable (VL) domain comprising an LCDR1 amino acid sequence of GSSTGAVTTSNYAN (SEQ ID NO: 41), an LCDR2 amino acid sequence of GTNKRAP (SEQ ID NO: 42), and an LCDR3 amino acid sequence of ALWYSNHWV (SEQ ID NO: 43).

33. The multispecific antigen binding protein of claim 32, wherein
(a) the VH of the CD3 binding domain comprises or consists of a heavy chain domain comprising an amino acid sequence that is at least about 90%, 95%, 96%, 97%, 98%, 99% or 100% identical to the amino acid sequence of SEQ ID NO: 36, and/or
(b) the VL of the CD3 binding domain comprises or consists of an amino acid sequence that is at least about 90%, 95%, 96%, 97%, 98%, 99% or 100% identical to the amino acid sequence of SEQ ID NO: 37.

34. The multispecific antigen binding protein of claim 22, wherein the immune cell binding domain is or comprises a Fab fragment, said Fab fragment comprising:
(1) a heavy chain (HC) comprising a CH1 domain and the VH; and
(2) a light chain (LC) comprising a CL domain and the VL.

35. The multispecific antigen binding protein of claim 34, wherein:
(1) the HC comprises or consists of an amino acid sequence that is at least about 90%, 95%, 96%, 97%, 98%, 99% or 100% identical to the amino acid sequence of SEQ ID NO: 34; and
(2) the LC comprises or consists of an amino acid sequence that is at least about 90%, 95%, 96%, 97%, 98%, 99% or 100% identical to the amino acid sequence of SEQ ID NO: 35.

36. The multispecific antigen binding protein of claim 34, wherein:
the CH1 domain comprises five amino acids of an antibody hinge region, in particular an amino acid sequence EPKSC (SEQ ID NO: 17);
the first antigen binding domain is operably linked to the C-terminus of the heavy chain or the N-terminus of the heavy chain of the Fab fragment;
the second antigen binding domain is operably linked to the C-terminus of the light chain or the N-terminus of the light chain of the Fab fragment;
a) the first antigen binding domain is or comprises an scFv being linked to the C-terminus of the Fab domain heavy chain and the second antigen binding domain comprises an scFv being linked to the C-terminus of the Fab domain light chain; b) the first antigen binding domain is or comprises an scFv being linked to the N-terminus of the Fab domain heavy chain and the second antigen binding domain comprises an scFv being linked to the N-terminus of the Fab domain light chain; c) the first antigen binding domain is or comprises an scFv being linked to the N-terminus of the Fab domain heavy chain and the second antigen binding domain comprises an scFv being linked to the C-terminus of the Fab domain light chain; or d) the first antigen binding domain is or comprises an scFv being linked to the C-terminus of the Fab domain heavy chain and the second antigen binding domain comprises an scFv being linked to the N-terminus of the Fab domain light chain;
the scFv is linked to the Fab domain with an amino acid linker, optionally wherein the amino acid linker comprises GGGGS (SEQ ID NO: 2; and/or
the multispecific antigen binding protein does not comprise an Fc domain.

37-42. (canceled)

43. The multispecific antigen binding protein of claim 22, being a Fab-scFv, comprising:
the scFv of SEQ ID NO: 33, being linked to the C-terminus of the HC of SEQ ID NO: 34; and a LC of SEQ ID NO: 35;
or variants of said sequences that are at least about 90%, 95%, 96%, 97%, 98% or 99% identical to the amino acid sequences.

44. The multispecific antigen binding protein of claim 22, being a bispecific Fab-scFv₂ comprising:
(i) a HC comprising or consisting of the amino acid sequence of SEQ ID NO: 52, or a variant thereof that is at least about 90%, 95%, 96%, 97%, 98%, or 99% identical thereto; and
(ii) a LC comprising or consisting of the amino acid sequence of SEQ ID NO: 53, or a variant thereof that is at least about 90%, 95%, 96%, 97%, 98%, or 99% identical thereto.

45. The multispecific antigen binding protein of claim 22, wherein:
the light chain and/or heavy chain comprises an N-terminal and/or C-terminal truncation of 1, 2, 3, 4, or 5 amino acids, optionally wherein the light chain comprises an N-terminal truncation of 1 or 2 amino acids; and/or
comprising a pyroglutamate (pE) at position 1 instead of glutamine (Q) or glutamate (E) of the light chain and/or heavy chain, optionally comprising a pyroglutamate (pE) at position 1 instead of glutamine (Q) or glutamate (E) of the light chain.

46-53. (canceled)

54. A kit comprising the antigen binding protein of claim 1.

55-58. (canceled)

59. A pharmaceutical composition comprising antigen binding protein of claim 1, and a pharmaceutically acceptable buffer.

60-66. (canceled)
